SEQUENCE LISTING (64 sequences)

SEQ ID NO: 1 - DNA, 40 bp, Artificial Sequence
  Synthetic Polynucleotide
tttggtctagctacagtgaaatctcgatggagtgggtccc

SEQ ID NO: 2 - DNA, 40 bp, Artificial Sequence
  Synthetic Polynucleotide
tttggtctagctacagagaaatctcgatggagtgggtccc

SEQ ID NO: 3 - DNA, 40 bp, Artificial Sequence
  Synthetic Polynucleotide
tttggtctagctacagaaaaatctcgatggagtgggtccc

SEQ ID NO: 4 - DNA, 36 bp, Artificial Sequence
  Synthetic Polynucleotides
acgccaccagctccaactaccacaagtttatattca

SEQ ID NO: 5 - DNA, 37 bp, Artificial Sequence
  Synthetic Polynucleotides
cgccacaagctccaactaccacaagtttatattcagt

SEQ ID NO: 6 - DNA, 37 bp, Artificial Sequence
  Synthetic Polynucleotides
acgccactagctccaactaccacaagtttatattcag

SEQ ID NO: 7 - DNA, 36 bp, Artificial Sequence
  Synthetic Polynucleotides
cgccacgagctccaactaccacaagtttatattcag

SEQ ID NO: 8 - DNA, 37 bp, Artificial Sequence
  Synthetic Polynucleotides
tacgccaacagctccaactaccacaagtttatattca

SEQ ID NO: 9 - DNA, 37 bp, Artificial Sequence
  Synthetic Polynucleotides
tacgccatcagctccaactaccacaagtttatattca

SEQ ID NO: 10 - DNA, 36 bp, Artificial Sequence
  Synthetic Polynucleotides
acgccagcagctccaactaccacaagtttatattca

SEQ ID NO: 11 - DNA, 37 bp, Artificial Sequence
  Synthetic Polynucleotides
cgcaaccagctccaactaccacaagtttatattcagt

SEQ ID NO: 12 - DNA, 38 bp, Artificial Sequence
  Synthetic Polynucleotides
tacgctaccagctccaactaccacaagtttatattcag

SEQ ID NO: 13 - DNA, 36 bp, Artificial Sequence
  Synthetic Polynucleotides
cgcgaccagctccaactaccacaagtttatattcag

SEQ ID NO: 14 - DNA, 37 bp, Artificial Sequence
  Synthetic Polynucleotides
tacgtcaccagctccaactaccacaagtttatattca

SEQ ID NO: 15 - DNA, 35 bp, Artificial Sequence
  Synthetic Polynucleotides
ttctgaattagctgtatcgtcaaggcactcttgcc

SEQ ID NO: 16 - DNA, 42 bp, Artificial Sequence
  Synthetic Polynucleotides
ctcttaattccttgatagcgacgggaattttaactttctcac

SEQ ID NO: 17 - DNA, 39 bp, Artificial Sequence
  Synthetic Polynucleotide
ggagatgttttgatagcgacgggaattttaactttctca

SEQ ID NO: 18 - DNA, 38 bp, Artificial Sequence
  Synthetic Polynucleotide
gagatgtcttgatagcgacgggaattttaactttctca

SEQ ID NO: 19 - DNA, 39 bp, Artificial Sequence
  Synthetic Polynucleotide
ggagattccttgatagcgacgggaattttaactttctca

SEQ ID NO: 20 - DNA, 31 bp, Artificial Sequence
  Synthetic Polynucleotide
tcacgggccttgtacactgtcccataggcac

SEQ ID NO: 21 - DNA, 32 bp, Artificial Sequence
  Synthetic Polynucleotide
tcacaggccttgtacactgtcccataggcacc

SEQ ID NO: 22 - DNA, 35 bp, Artificial Sequence
  Synthetic Polynucleotide
acactcttgagggccacaaagtggccactgtgggg

SEQ ID NO: 23 - DNA, 34 bp, Artificial Sequence
  Synthetic Polynucleotide
tggagactcctttcaattgactgtcaccagcccc

SEQ ID NO: 24 - DNA, 33 bp, Artificial Sequence
  Synthetic Polynucleotide
gtgaagactcctttcaattgactgtcaccagcc

SEQ ID NO: 25 - DNA, 36 bp, Artificial Sequence
  Synthetic Polynucleotide
aggatagaagcagtttccaacatttcagccatgaac

SEQ ID NO: 26 - DNA, 33 bp, Artificial Sequence
  Synthetic Polynucleotide
catcgaagccgtccatgaggaagaggattctgg

SEQ ID NO: 27 - DNA, 34 bp, Artificial Sequence
  Synthetic Polynucleotide
tcattgaagccgtccatgaggaagaggattctgg

SEQ ID NO: 28 - DNA, 35 bp, Artificial Sequence
  Synthetic Polynucleotide
gcggtcctatgtgctcgtcaaaggcaccttgcagc

SEQ ID NO: 29 - DNA, 30 bp, Artificial Sequence
  Synthetic Polynucleotide
tcaggcggggctatccgaagaccctgggac

SEQ ID NO: 30 - DNA, 31 bp, Artificial Sequence
  Synthetic Polynucleotide
tcaagcggggctatccgaagaccctgggaca

SEQ ID NO: 31 - DNA, 35 bp, Artificial Sequence
  Synthetic Polynucleotide
acccctcgccaggccaggcacataccacgggcgct

SEQ ID NO: 32 - DNA, 31 bp, Artificial Sequence
  Synthetic Polynucleotide
gccgcccatgcaggaactgttacacatgtag

SEQ ID NO: 33 - DNA, 33 bp, Artificial Sequence
  Synthetic Polynucleotide
gctgcccatgcaggaactgttacacatgtagtt

SEQ ID NO: 34 - DNA, 35 bp, Artificial Sequence
  Synthetic Polynucleotide
agtgtgatgatggtgaggatgggcctccggttcat

SEQ ID NO: 35 - DNA, 42 bp, Artificial Sequence
  Synthetic Polynucleotide
cgagatttcactgtagctagaccaaaatcacctatttttact

SEQ ID NO: 36 - DNA, 42 bp, Artificial Sequence
  Synthetic Polynucleotide
cgagatttctctgtagctagaccaaaatcacctatttttact

SEQ ID NO: 37 - DNA, 35 bp, Artificial Sequence
  Synthetic Polynucleotide
ccagacaactgttcaaactgatgggacccactcca

SEQ ID NO: 38 - DNA, 37 bp, Artificial Sequence
  Synthetic Polynucleotide
gtactcttcttgtccagctgtatccagtatgtccaac

SEQ ID NO: 39 - DNA, 35 bp, Artificial Sequence
  Synthetic Polynucleotide
actcttctcgtccagctgtatccagtatgtccaac

SEQ ID NO: 40 - DNA, 35 bp, Artificial Sequence
  Synthetic Polynucleotide
tcgcctgtcctcatgtattggtctctcatggcact

SEQ ID NO: 41 - DNA, 24 bp, Artificial Sequence
  Synthetic Polynucleotide
agatagaagtttggagagagaacg

SEQ ID NO: 42 - DNA, 24 bp, Artificial Sequence
  Synthetic Polynucleotide
ggtatgaatggctgacacttcttc

SEQ ID NO: 43 - DNA, 25 bp, Artificial Sequence
  Synthetic Polynucleotide
gcctcaattcttaccatccacaaaa

SEQ ID NO: 44 - DNA, 25 bp, Artificial Sequence
  Synthetic Polynucleotide
ggtgtcaggaaaatgctggctgacc

SEQ ID NO: 45 - DNA, 22 bp, Artificial Sequence
  Synthetic Polynucleotide
tgatggccagcgtggacaaccc

SEQ ID NO: 46 - DNA, 22 bp, Artificial Sequence
  Synthetic Polynucleotide
tgcgatctgcacacaccagttg

SEQ ID NO: 47 - DNA, 24 bp, Artificial Sequence
  Synthetic Polynucleotide
ggtgcacgaagggccagggtatgt

SEQ ID NO: 48 - DNA, 24 bp, Artificial Sequence
  Synthetic Polynucleotide
gtacctcgggcacagggcttgctg

SEQ ID NO: 49 - DNA, 24 bp, Artificial Sequence
  Synthetic Polynucleotide
gaaagggcccaaattcaccaataa

SEQ ID NO: 50 - DNA, 25 bp, Artificial Sequence
  Synthetic Polynucleotide
ggatctacagatcgggacactcaaa

SEQ ID NO: 51 - DNA, 24 bp, Artificial Sequence
  Synthetic Polynucleotide
atggcaaatacacaaagaaagccc

SEQ ID NO: 52 - DNA, 24 bp, Artificial Sequence
  Synthetic Polynucleotide
ttctcccttctcaggattcctaca

SEQ ID NO: 53 - DNA, 24 bp, Artificial Sequence
  Synthetic Polynucleotide
ttgttggatcatattcgtccacaa

SEQ ID NO: 54 - DNA, 25 bp, Artificial Sequence
  Synthetic Polynucleotide
aaccttatgtgtgacatgttctaat

SEQ ID NO: 55 - DNA, 24 bp, Artificial Sequence
  Synthetic Polynucleotide
ttgatggcaaatacacagaggaag

SEQ ID NO: 56 - DNA, 21 bp, Artificial Sequence
  Synthetic Polynucleotide
tccacacccccaggattctta

SEQ ID NO: 57 - DNA, 24 bp, Artificial Sequence
  Synthetic Polynucleotide
tcgaaagaccctagccttagataa

SEQ ID NO: 58 - DNA, 24 bp, Artificial Sequence
  Synthetic Polynucleotide
ccagagtgagctttcattttctca

SEQ ID NO: 59 - DNA, 24 bp, Artificial Sequence
  Synthetic Polynucleotide
ggccagtgtgcagggtggcaagtg

SEQ ID NO: 60 - DNA, 24 bp, Artificial Sequence
  Synthetic Polynucleotide
tggcctcatcttgggcctgtgtta

SEQ ID NO: 61 - DNA, 35 bp, Artificial Sequence
  Synthetic Polynucleotide
gctatcaaggaattaagagaagcaacatctccgaa

SEQ ID NO: 62 - DNA, 20 bp, Artificial Sequence
  Synthetic Polynucleotide
gctatcaaaacatctccgaa

SEQ ID NO: 63 - DNA, 21 bp, Artificial Sequence
  Synthetic Polynucleotide
gctatcaagacatctcccgaa

SEQ ID NO: 64 - DNA, 20 bp, Artificial Sequence
  Synthetic Polynucleotide
gctatcaaggaatctccgaa

(19) United States
(12) Patent Application Publication
(10) Pub. No.: US 2017/0081713 A1
(43) Pub. Date: Mar. 23, 2017

(54) MULTIVALENT PROBES HAVING SINGLE NUCLEOTIDE RESOLUTION

(71) Applicant: NanoString Technologies, Inc., Seattle, WA (US)

(72) Inventors: Dae KIM, Bellevue, WA (US); Paul Martin ROSS, Lake Forest Park, WA (US); Gavin MEREDITH, Seattle, WA (US); Elizabeth A. MANRAO, Lake Forest Park, WA (US)

(21) Appl. No.: 15/257,213

(22) Filed: Sep. 6, 2016

Related U.S. Application Data
(60) Provisional application No. 62/292,690, filed on Feb. 8, 2016.
(60) Provisional application No. 62/213,812, filed on Sep. 3, 2015.

(51) Int. Cl. C12Q 1/68

(52) U.S. Cl. CPC C12Q 1/6827

(57) ABSTRACT

The present invention relates to, among other things, polymer strands, probes, compositions, methods, and kits for enabling accurate and robust enzyme- and amplification-free detection of DNA and RNA with single base resolution (e.g., detection of a single nucleotide polymorphism (SNP), an insertion, and a deletion). The compositions, methods, and kits may further provide simultaneous detection of DNA and/or RNA and protein targets.

### CROSS-REFERENCE TO RELATED APPLICATIONS

This application is claims priority to and the benefit of U.S. Provisional Application No. 62/213,812, filed Sep. 3, 2015 and U.S. Provisional Application No. 62/292,690, filed Feb. 8, 2016. Each of the above-mentioned applications is incorporated herein by reference in its entirety.

### SEQUENCE LISTING

The instant application contains a Sequence Listing which has been submitted in ASCII format via EFS-Web and is hereby incorporated by reference in its entirety. Said ASCII copy, created on Oct. 24, 2016 is named NATE-029001US_ST25.txt and is 13,070 bytes in size.

### BACKGROUND OF THE INVENTION

In nucleic acid detection, a trade-off exists between probes having high stability and probes having high specificity; for example, longer length probes have high melting temperatures and are highly stable, however, they lack specificity and are unable to detect single base substitutions. There exists a need for probes, compositions, methods, and kits for enabling accurate and robust enzyme- and amplification-free detection of DNA and RNA and with single base resolution.

### SUMMARY OF THE INVENTION

The present invention relates to polymer strands, probes, compositions, methods, and kits for enabling accurate and robust enzyme- and amplification-free detection of DNA and RNA with single base resolution (e.g., detection of a single nucleotide polymorphism (SNP), an insertion, and a deletion).

A first aspect of the present invention relates to a polymer strand pair including a first polymer strand having at least (1) a first target binding region, (2) a first complementary region, and (3) a sequence-specific region and a second polymer strand including at least (1) a second target binding region and (2) a second complementary region. The target of the first target binding region and the target of the second target binding region are in the same nucleic acid molecule and the target of the first target binding region is non-overlapping with the target of the second target binding region. The first complementary region is complementary to the second complementary region.

A second aspect of the present invention relates to a method for detecting a nucleic acid in a sample including a step of contacting the sample with a polymer strand pair of the first aspect. This aspect includes a step of detecting a linear combination of labelled monomers or detecting one or more label monomers, thereby detecting the nucleic acid molecule in the sample.

In each aspect of the present invention that is directed to detecting a nucleic acid in a sample, each first polymer strand has a sequence-specific region that (a) includes at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (b) is covalently attached to a single-stranded nucleic acid backbone, the backbone including at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (c) is bound or capable of being bound to a reporter probe, the reporter probe including at least a binding portion complementary to the sequence-specific region and a single-stranded nucleic acid backbone, the backbone including at least two label attachment positions covalently linked in a linear combination, with each label attachment position capable of binding at least one complementary single-stranded oligonucleotide including at least one label monomer, in which a linear combination of labelled monomers identifies the nucleic acid molecule; or (d) includes one or more label monomers, with the one or more label monomers identifying the nucleic acid molecule. The one or more label monomer is selected from biotin, chemiluminescent marker, dye, enzyme, fluorochrome, nanoparticle, quantum dot, and another monomer that can be detected directly or indirectly.

A third aspect of the present invention relates to a composition including a plurality of polymer strand pairs of the first aspect. In this aspect, a first polymer strand pair is capable of binding to a first nucleic acid molecule and an at least second polymer strand pair is capable of binding to an at least second nucleic acid molecule. The first nucleic acid molecule differs from the at least second nucleic acid molecule.

A fourth aspect of the present invention relates to a method for detecting a plurality of nucleic acids in a sample including a step of contacting the sample with a plurality of polymer strand pairs of the first aspect or of contacting the sample with a composition of the third aspect. This aspect includes a step of (1) detecting a linear combination of labelled monomers for a first polymer strand pair or detecting one or more label monomers on a first polymer strand pair and (2) detecting a linear combination of labelled monomers for an at least second polymer strand pair or detecting one more label monomers on an at least second polymer strand pair, thereby detecting the first nucleic acid molecule and the at least second nucleic acid molecule in the sample.

A fifth aspect of the present invention relates to a polymer strand trio including a polymer strand pair of the first aspect and a capture polymer strand. The capture polymer strand includes at least (1) a region having at least one affinity moiety or a region capable of binding to a single-stranded nucleic acid including at least one affinity moiety and (2) a third target binding region capable of binding to the nucleic acid molecule. The targets of the first, second, and third target binding regions are non-overlapping and in the same nucleic acid molecule.

A sixth aspect of the present invention relates to a method for detecting a nucleic acid in a sample including a step of contacting the sample with a polymer strand trio of the fifth aspect. This aspect includes a step of detecting a linear combination of labelled monomers for the polymer strand trio or detecting one or more label monomers on the polymer strand trio, thereby detecting the nucleic acid molecule in the sample.

A seventh aspect of the present invention relates to a composition including a plurality of polymer strand trios of the fifth aspect. In this aspect, a first polymer strand trio is capable of binding to a first nucleic acid molecule and an at least second polymer strand trio is capable of binding to an at least second nucleic acid molecule. The first nucleic acid molecule differs from the at least second nucleic acid molecule.

An eight aspect of the present invention relates to a method for detecting a plurality of nucleic acids in a sample including a step of contacting the sample with a plurality of polymer strand trios of the fifth aspect or of contacting the sample with a composition of the seventh aspect. This aspect includes a step of (1) detecting a linear combination of labelled monomers for a first polymer strand trio or detecting one or more label monomers on a first polymer strand trio and (2) detecting a linear combination of labelled monomers for an at least second polymer strand trio or detecting one or more label monomers on an at least second polymer strand trio, thereby detecting the first nucleic acid molecule and the at least second nucleic acid molecule in the sample.

A ninth aspect of the present invention relates to a partially double-stranded nucleic acid probe obtained when the first complementary region and the second complementary region of a polymer strand pair of the first aspect are hybridized. Upon hybridization, the first polymer strand and the second polymer strand form a partially-double stranded nucleic acid probe having each feature of the polymer strand pair of the first aspect.

A tenth aspect of the present invention relates to a method for detecting a nucleic acid in a sample including a step of contacting the sample with a partially double-stranded nucleic acid probe of the ninth aspect. This aspect includes a step of detecting a linear combination of labelled monomers for the partially double-stranded nucleic acid probe or detecting one or more label monomers on the partially double-stranded nucleic acid probe, thereby detecting the nucleic acid molecule in the sample.

An eleventh aspect of the present invention relates to a composition including a plurality of partially double-stranded nucleic acid probes of the ninth aspect. In this aspect, a first double-stranded nucleic acid probe is capable of binding to a first nucleic acid molecule and an at least second double-stranded nucleic acid probe is capable of binding to an at least second nucleic acid molecule. The first nucleic acid molecule differs from the at least second nucleic acid molecule.

A twelfth aspect of the present invention relates to a method for detecting a plurality of nucleic acids in a sample including a step of contacting the sample with a plurality of partially double-stranded nucleic acid probes of the ninth aspect or of contacting the sample with a composition of the eleventh aspect. This aspect includes a step of (1) detecting a linear combination of labelled monomers for a first partially double-stranded nucleic acid probe or detecting one or more label monomers on a first partially double-stranded nucleic acid probe and (2) detecting a linear combination of labelled monomers for an at least second partially double-stranded nucleic acid probe or detecting one or more label monomers on an at least second partially double-stranded nucleic acid probe, thereby detecting the first nucleic acid molecule and the at least second nucleic acid molecule in the sample.

A thirteenth aspect of the present invention relates to a composition including a plurality of partially double-stranded nucleic acid probes of the ninth aspect and a plurality of capture polymer strands. A first capture polymer strand at least includes (1) a region including at least one affinity moiety or a region capable of binding to a single-stranded nucleic acid including at least one affinity moiety and (2) a third target binding region that is capable of binding to a first nucleic acid molecule. Each at least second capture polymer strand at least includes (1) a region including at least one affinity moiety or a region capable of binding to a single-stranded nucleic acid comprising at least one affinity moiety and (2) a third target binding region that is capable of binding to an at least second nucleic acid molecule. The targets of each first, second, and third target binding regions are non-overlapping and in the same nucleic acid molecule. The first nucleic acid molecule differs from the at least second nucleic acid molecule.

A fourteenth aspect of the present invention relates to a multivalent polymer strand including at least (1) a first target binding region, (2) a second target binding region, (3) a spacer between the first target binding region and the second target binding region, and (4) a sequence-specific region. The target of the first target binding region and the target of the second target binding region are in the same nucleic acid molecule and the target of the first target binding region is non-overlapping with the target of the second target binding region. The spacer may be polymer chain, e.g., an oligonucleotide and polyethylene glycol.

A fifteenth aspect of the present invention relates to a method for detecting a nucleic acid in a sample including a step of contacting the sample with a multivalent polymer strand of the fourteenth aspect. This aspect includes a step of detecting a linear combination of labelled monomers or detecting one or more label monomers, thereby detecting the nucleic acid molecule in the sample.

A sixteenth aspect of the present invention relates to a composition including a plurality of multivalent polymer strands of the fourteenth aspect. In this aspect, a first multivalent polymer strand is capable of binding to a first nucleic acid molecule and an at least second multivalent polymer strand is capable of binding to an at least second nucleic acid molecule. The first nucleic acid molecule differs from the at least second nucleic acid molecule.

A seventeenth aspect of the present invention relates to a method for detecting a plurality of nucleic acids in a sample including a step of contacting the sample with a plurality of multivalent polymer strands of the fourteenth aspect or of contacting the sample with a composition of the sixteenth aspect. This aspect includes a step of (1) detecting a linear combination of labelled monomers for a first multivalent polymer strand or detecting one or more label monomers on a first multivalent polymer strand and (2) detecting a linear combination of labelled monomers for an at least second multivalent polymer strand or detecting one or more label monomers on an at least second multivalent polymer strand, thereby detecting the first nucleic acid molecule and the at least second nucleic acid molecule in the sample.

An eighteenth aspect of the present invention relates to a multivalent polymer strand duo including a multivalent polymer strand of the fourteenth aspect and a capture polymer strand. The capture polymer strand includes at least (1) a region having at least one affinity moiety or a region capable of binding to a single-stranded nucleic acid including at least one affinity moiety and (2) a third target binding region capable of binding to the nucleic acid molecule. The targets of the first, second, and third target binding regions are non-overlapping and in the same nucleic acid molecule.

A nineteenth aspect of the present invention relates to a method for detecting a nucleic acid in a sample including a step of contacting the sample with a multivalent polymer strand duo of the eighteenth aspect. This aspect includes a step of detecting a linear combination of labelled monomers for the polymer strand trio or detecting one or more label monomers on the polymer strand trio thereby detecting the nucleic acid molecule in the sample.

A twentieth aspect of the present invention relates to a composition including a plurality of multivalent polymer strand duos of the eighteenth aspect. In this aspect, a first multivalent polymer strand duo is capable of binding to a first nucleic acid molecule and an at least second multivalent polymer strand duo is capable of binding to an at least second nucleic acid molecule. The first nucleic acid molecule differs from the at least second nucleic acid molecule.

A twenty-first aspect of the present invention relates to a method for detecting a plurality of nucleic acids in a sample including a step of contacting the sample with a plurality of multivalent polymer strand duos of the eighteenth aspect or of contacting the sample with a composition of the twentieth aspect. This aspect includes a step of (1) detecting a linear combination of labelled monomers for a first polymer strand trio or detecting one or more label monomers on a first polymer strand trio and (2) detecting a linear combination of labelled monomers for an at least second polymer strand trio or one or more label monomers on an at least second polymer strand trio, thereby detecting the first nucleic acid molecule and the at least second nucleic acid molecule in the sample.

Any of the herein-described compositions may further comprise at least one probe capable of detecting a protein target.

Any of the herein-described methods may further comprise contacting a sample with at least one probe capable of detecting a protein target.

A twenty-second aspect of the present invention relates to a kit comprising a composition of the third aspect, of the seventh aspect, of the eleventh aspect, of the thirteenth aspect, of the sixteenth aspect, or of the twentieth aspect and instructions for use. Other components necessary to perform a method of any of the above aspects may be included in a kit. The kit may further comprise at least one probe capable of detecting a protein target.

In each aspect of the present invention, a labeled oligonucleotide may be labeled with one or more detectable label monomers. The label may be at a terminus of an oligonucleotide, at a point within an oligonucleotide, or a combination thereof. Oligonucleotides may comprise nucleotides with amine-modifications, which allow coupling of a detectable label to the nucleotide. Labeled oligonucleotides of the present invention can be labeled with any of a variety of label monomers, such as a fluorochrome, quantum dot, dye, enzyme, nanoparticle, chemiluminescent marker, biotin, or other monomer known in the art that can be detected directly (e.g., by light emission) or indirectly (e.g., by binding of a fluorescently-labeled antibody). Preferred examples of a label that can be utilized by the invention are fluorophores. Several fluorophores can be used as label monomers for labeling nucleotides including, but not limited to GFP-related proteins, cyanine dyes, fluorescein, rhodamine, ALEXA Fluor™, Texas Red, FAM, JOE, TAN/IRA, and ROX. Several different fluorophores are known, and more continue to be produced, that span the entire spectrum.

In each aspect of the present invention, a label attachment position may be hybridized (non-covalently bound) with at least one labeled oligonucleotide. Alternately, a position may be hybridized with at least one oligonucleotide lacking a detectable label. Each position can hybridize to 1, 2, 3, 4, 5, 6, 7, 8, 9, 10, 11, 12, 13, 14, 15, 16, 17, 18, 19, 20, or 21 to 100 labeled (or unlabeled) oligonucleotides or more. The number of labeled oligonucleotides hybridized to each position depends on the length of the position and the size of the oligonucleotides. A position may be between about 12 to about 1500 nucleotides in length. The lengths of the labeled (or unlabeled) oligonucleotides vary from about 12 to about 1500 nucleotides in length. In embodiments, the lengths of labeled (or unlabeled) oligonucleotides vary from about 800 to about 1300 ribonucleotides. In other embodiments, the lengths of labeled (or unlabeled) oligonucleotides vary from about 20 to about 55 deoxyribonucleotides; such oligonucleotides are designed to have melting/hybridization temperatures of between about 65 and about 85° C., e.g., about 80° C. For example, a position of about 1100 nucleotides in length may hybridize to between about 25 and about 45 oligonucleotides comprising, each oligonucleotide about 45 to about 25 deoxyribonucleotides in length. In embodiments, each position is hybridized to about 34 labeled oligonucleotides of about 33 deoxyribonucleotides in length. The labeled oligonucleotides are preferably single-stranded DNA.

In each aspect of the present invention, labels associated with each position (via hybridization of a position with a labeled oligonucleotide) are spatially-separable and spectrally-resolvable from the labels of a preceding position or a subsequent position. An ordered series of spatially-separable and spectrally-resolvable labels of a probe is herein referred to as barcode or as a label code. The barcode or label code allows identification of a target nucleic acid or target protein that has been bound by a particular probe.

The terms “one or more”, “at least one”, and the like are understood to include but not be limited to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between. Therefore, an “at least one complementary single-stranded oligonucleotide” may include, for example, 2 oligonucleotides, 6 oligonucleotides, and 10 oligonucleotides.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between. Therefore, an “at least second polymer strand pair” includes, but is not limited to, 2 polymer strand pairs, 10 polymer strand pairs, 100 polymer strand pairs, and 1000 polymer strand pairs. Similarly, “at least second nucleic acid molecule” includes, but is not limited, to 2 nucleic acid molecules, 20 nucleic acid molecules, 40 nucleic acid molecules, and 60 nucleic acid molecules. Also, “at least second capture polymer strand” includes, but is not limited, to 2 capture polymer strands, 500 capture polymer strands, 1000 capture polymer strands, and 5000 capture polymer strands. Moreover, “at least two label attachment positions” includes, but is not limited, 2 label attachment positions, 4 label attachment positions, 6 label attachment positions, and 8 label attachment positions.

In embodiments, 1, 2, 3, 4, 5, 6, 7, 8, 9, 10, 11, 12, 13, 14, 15, 16, 17, 18, 19, 20, 30, 40, 50, 60, 70, 80, 90, 100, 200, 300, 400, 500, 600, 700, 800, 900, 1000 or more nucleic acids, and any number there between, are detected. 800 or more different nucleic acids can be detected. In embodiments, detecting includes quantifying the abundance of each nucleic acid.

The probes and methods disclosed herein permit detection of somatic variants with about 5% allele frequency from as little as 5 ng fresh or formalin-fixed paraffin embedded (FFPE) genomic DNA (gDNA).

nCounter® probes, systems, and methods from NanoString Technologies®, as described in US2003/0013091, US2007/0166708, US2010/0015607, US2010/0261026, US2010/0262374, US2010/0112710, US2010/0047924, US2014/0371088, US2014/0017688, and US2011/0086774), are a preferred means for identifying target proteins and/or target nucleic acids. nCounter® probes, systems, and methods from NanoString Technologies® allow simultaneous multiplexed identification a plurality (800 or more) distinct target proteins and/or target nucleic acids. Each of the above-mentioned patent publications is incorporated herein by reference in its entirety. The above-mentioned nCounter® probes, systems, and methods from NanoString Technologies® can be combined with any aspect or embodiment described herein.

A single nCounter® cartridge (e.g., a single lane thereof) may be used for simultaneous multiplexed identification of a plurality distinct target proteins and/or target nucleic acids from the combination of the above-mentioned nCounter® probes, systems, and methods and the aspects or embodiments described herein.

Any aspect or embodiment described herein can be combined with any other aspect or embodiment as disclosed herein.

While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A to FIG. 1G show exemplary polymer strands, polymer strand pairs, and partially double-stranded nucleic acid probes. FIG. 1A shows a polymer strand pair comprising a first polymer strand including a first target binding region (in red) and a second target binding region (in green). FIG. 1B shows a polymer strand pair having a first polymer strand comprising a label moiety (green circle) and a second polymer strand having an affinity moiety (asterisk). FIG. 1C shows a polymer strand pair in which the first polymer strand is covalently attached to a single-stranded nucleic acid backbone including a plurality (six shown) of label attachment positions covalently linked in a linear combination with each label attachment position bound by at least one complementary single-stranded oligonucleotide comprising at least one label monomer; alternately, the sequence-specific region includes a plurality of label attachment positions covalently linked in a linear combination with each label attachment position bound by at least one complementary single-stranded oligonucleotide comprising at least one label monomer. FIG. 1D through FIG. 1G show polymer strand pairs in which each first complementary region is hybridized to a second complementary region, thereby producing partially double-stranded nucleic acid probes. FIG. 1F shows a polymer strand pair/partially double-stranded nucleic acid probe in which one of the plurality of label attachment positions is bound by complementary single-stranded oligonucleotides lacking label monomers (shown as open black circle). FIG. 1G shows a polymer strand pair/double-stranded nucleic acid probe having a sequence-specific region that is bound to a reporter probe, with the reporter probe including a plurality (five shown) of label attachment positions each bound by at least one complementary single-stranded oligonucleotide comprising at least one label monomer. Each colored circle represents the totality of label monomers associated with each label attachment position or associated with a sequence-specific region. The colors shown in FIG. 1A to FIG. 1G, and elsewhere in this disclosure, are non-limiting; other colored labels and other detectable labels known in the art can be used in the probes of the present invention.

FIG. 2A to FIG. 2C show polymer strand pairs/double-stranded nucleic acid probes similar to those in FIG. 1A to FIG. 1G each bound to a nucleic acid molecule. FIG. 2B shows a capture polymer strand bound to the nucleic acid; the capture polymer strand includes an affinity moiety (asterisk).

FIG. 3A to FIG. 311 show exemplary polymer strands, polymer strand pairs, and partially double-stranded nucleic acid probes each including at least one spacer (shown as a blue curvilinear line). FIG. 3A shows a polymer strand pair in which each polymer strand has a spacer. FIG. 3B shows a polymer strand pair in which the first polymer strand has a spacer. FIG. 3C shows a polymer strand pair in which the second polymer strand has a spacer. FIG. 3E shows a polymer strand pair/double-stranded nucleic acid probe in which the first polymer strand includes an affinity moiety (asterisk). FIG. 3F shows a polymer strand pair/partially double-stranded nucleic acid probe in which one of the plurality of label attachment positions is bound by complementary single-stranded oligonucleotides lacking label monomers (shown as open black circle).

FIG. 4A to FIG. 4C show polymer strand pairs/double-stranded nucleic acid probes similar to those in FIG. 3A to FIG. 3E each bound to a nucleic acid molecule. FIG. 4C shows a capture polymer strand bound to the nucleic acid; the capture polymer strand is in turn bound by a single-stranded nucleic acid including at least one affinity moiety (asterisk).

FIG. 5A to FIG. 5C show certain steps for detecting a nucleic acid molecule using the polymer strands, polymer strand pairs, and partially double-stranded nucleic acid probes of the present invention. FIG. 5A shows binding of a first polymer strand to the nucleic acid molecule; the first polymer strand includes a spacer. FIG. 5B shows a later step in which a second polymer strand has been bound to the nucleic acid molecule and the first complementary region is hybridized to a second complementary region. Note that the step of FIG. 5B may be the first step in the method in that the first and second polymer strands are simultaneously provided to a nucleic acid molecule; alternately, the first and second polymers strands may be first hybridized via their complementary regions (thereby producing a partially double-stranded nucleic acid probe), and then the probe is provided to the nucleic acid molecule. FIG. 5C shows the double-stranded nucleic acid probe of FIG. 5B having its sequence-specific region bound to a reporter probe, with the reporter probe including a plurality (four shown) of label attachment positions each bound by at least one complementary single-stranded oligonucleotide comprising at least one label monomer. Note that each component of the complex shown in FIG. 5C can be provided to a nucleic acid separately or simultaneously.

FIG. 6A and FIG. 6B show other steps for detecting a nucleic acid molecule using the polymer strands, polymer strand pairs, and partially double-stranded nucleic acid probes of the present invention. FIG. 6A shows a double-stranded nucleic acid probe and a capture polymer strand bound to the nucleic acid molecule. FIG. 6B shows the double-stranded nucleic acid probe of FIG. 6A with its sequence-specific region bound to a reporter probe, with the reporter probe including a plurality (six shown) of label attachment positions each bound by at least one complementary single-stranded oligonucleotide comprising at least one label monomer. The nucleic acid molecule is also bound by a capture polymer strand which is in turn bound by a single-stranded nucleic acid including at least one affinity moiety (asterisk).

FIG. 7A to FIG. 7E show another series of steps for detecting a nucleic acid molecule using the polymer strands, polymer strand pairs, and partially double-stranded nucleic acid probes of the present invention. FIG. 7A shows initial binding of a capture polymer strand to a nucleic acid molecule. Later, in FIG. 7C, a second polymer strand binds to the nucleic acid molecule.

FIG. 8A to FIG. 8F show exemplary polymer strands, polymer strand pairs, and partially double-stranded nucleic acid probes in which each first polymer strand includes a cleavable linker (shown as purple triangles). In FIG. 8C, the reporter probe includes an affinity moiety (asterisk) and in FIG. 8D, a portion of the sequence-specific region includes an affinity moiety.

FIG. 9A shows a partially double-stranded nucleic acid probe bound to a nucleic acid molecule in which the first polymer strand includes a cleavable linker (shown as a purple triangle). A force sufficient to cleave the cleavable linker is applied (red lightning bolt). FIG. 9B shows a portion of the sequence-specific region that is bound to a reporter probe that has been released from the partially double-stranded nucleic acid probe. The released reporter probe can then be detected. In this example, the portion of the sequence-specific region that is released includes an affinity moiety that can be captured in a subsequent step.

FIG. 10A to FIG. 10D show exemplary multivalent polymer strands. FIG. 10A shows a multivalent polymer strand including a first target binding region (green), a second target binding region (red), a spacer between the first target binding region and the second target binding region and a sequence-specific region (to the left of the second target binding region). FIG. 10B shows a multivalent polymer strand comprising a label moiety (red circle). FIG. 10C shows a multivalent polymer strand that is covalently attached to a single-stranded nucleic acid backbone including a plurality (six shown) of label attachment positions covalently linked in a linear combination with five of the label attachment positions bound by at least one complementary single-stranded oligonucleotide comprising at least one label monomer (colored circles) and one of the label attachment positions is bound by complementary single-stranded oligonucleotides lacking label monomers (shown as open black circle); alternately, the sequence-specific region includes a plurality of label attachment positions covalently linked in a linear combination with each label attachment position bound by at least one complementary single-stranded oligonucleotide. FIG. 10D shows a multivalent polymer strand having a sequence-specific region that is bound to a reporter probe, with the reporter probe including a plurality (six shown) of label attachment positions each bound by at least one complementary single-stranded oligonucleotide comprising at least one label monomer. Each colored circle represents the totality of label monomers associated with each label attachment position or associated with a sequence-specific region.

FIG. 11A to FIG. 11C show multivalent polymer strands similar to those in FIG. 10A to FIG. 10D each bound to a nucleic acid molecule. FIG. 11B shows a capture polymer strand bound to the nucleic acid; the capture polymer strand includes an affinity moiety (asterisk).

FIG. 12A to FIG. 12E show exemplary multivalent polymer strands each including one spacer (shown as a blue curvilinear line). FIG. 12B shows a multivalent polymer strand including an affinity moiety (asterisk). FIG. 12E shows a multivalent polymer strand in which one of the plurality of label attachment positions (of a reporter probe) is bound by complementary single-stranded oligonucleotides lacking label monomers (shown as open black circle).

FIG. 13A to FIG. 13C show multivalent polymer strands similar to those in FIG. 12A to FIG. 12E each bound to a nucleic acid molecule. FIG. 13C shows a multivalent polymer strand and a capture polymer strand bound to the nucleic acid molecule; the capture polymer strand is in turn bound by a single-stranded nucleic acid including at least one affinity moiety (asterisk).

FIG. 14A to FIG. 14D show exemplary steps for detecting a nucleic acid molecule using a multivalent polymer strand and a capture polymer strand.

FIG. 15A to FIG. 15F show exemplary multivalent polymer strands including a cleavable linker (shown as purple triangles). The multivalent polymer strands of FIG. 15D to FIG. 15F each include one spacer (shown as a blue curvilinear line) whereas the multivalent polymer strands of FIG. 15A to FIG. 15C lack a spacer.

FIG. 16 shows a graph illustrating melting temp distributions for a univalent probe and for polymer strand pairs/partially double-stranded probes/multivalent polymer strands of the present invention.

FIG. 17 outlines steps for detecting one or more nucleic acids in a sample usable in methods of the preset invention.

FIG. 18A illustrates polymer strand pairs/partially double-stranded probes used in the BRAF V600E SNP Detection experiments (Example 1) using polymer strand pairs/partially double-stranded probes with existing DV2 reporter probe from NanoString Technologies®. FIG. 18B shows the nucleic acid sequences that are bound by the three probes in FIG. 18A. FIG. 18C shows a subset of results obtained in Example 1.

FIG. 19 to FIG. 22 illustrate additional results obtained in the BRAF V600E SNP Detection experiments (Example 1).

FIG. 23 illustrates probe design trends for well-performing two-armed probes based upon results obtained in the BRAF V600E SNP Detection experiments (Example 1).

FIG. 24 provides results obtained in the EGFR T790M SNP Detection experiments (Example 1) using polymer strand pairs/partially double-stranded probes with existing DV2 reporter probe from NanoString Technologies®.

FIG. 25 illustrates probe design trends for well-performing two-armed probes based upon results obtained from four experiments. Further described in Example 1.

FIG. 26 shows a polymer strand pair/double-stranded nucleic acid probe similar to those in FIG. 1A to FIG. 3E bound to a nucleic acid molecule and a capture polymer strand bound to the nucleic acid; the capture polymer strand is in turn bound by a single-stranded nucleic acid including at least one affinity moiety. SNPs detectable by the polymer strand pair/double-stranded nucleic acid probe are shown.

FIG. 27 shows reference sequences for single nucleotide variants (SNV) in KRAS's exon 2, codons 12 and 13. Probes specific for the reference sequence and SNV loci were prepared and tested.

FIG. 28A to FIG. 28D show probes used in KRAS exon 2 Hotspot Experiment described in Example 2. Template sequence surrounding KRAS Exon 2 Hotspot shown along top. The matching reference probe and 10 SNV mutant probes are shown with the two target Binding Regions highlighted in blue and red. All experiments used a common Probe B oligo (red in FIG. 26). FIG. 28A shows the entire sequence and FIG. 28B to FIG. 28D each show one third of the sequence of FIG. 28A).

FIG. 29 shows results from the KRAS Exon 2 Hotspot Experiment described in Example 2. The specificity at each locus is determined by the percentage of digital counts for the probe exactly matching the target as a percentage of counts for all KRAS Exon 2 probes.

FIG. 30 shows EGFR's exon 19 and several of its known deletion variants.

FIG. 31A to FIG. 31D Probes used in EGFR Exon 19 Deletion Experiment of Example 3. Template sequence surrounding EGFR Exon 19 Deletions shown along top. The matching reference probe and three mutant probes are shown with the two target Binding Regions highlighted in blue and red. The deletion region is shown as a pink highlighted gap in the probe sequence. All experiments used a common Probe B oligo (red in FIG. 26). FIG. 31A shows the entire sequence and FIG. 31B to FIG. 31D each show one third of the sequence of FIG. 31A).

FIG. 32 shows results from the EGFR Exon 19 Deletion Experiment described in Example 3. The specificity for each deletion sequence is determined by the percentage of digital counts for the probe exactly matching the target as a percentage of counts for all EGFR Exon 19 probes.

FIG. 33A to FIG. 33D. Probes used in Multiplex SNV Experiment of Example 4. Template sequences surrounding each of the seven SNV loci are shown along top. The matching WT and SNV mutant probes are shown with the two target Binding Regions highlighted in blue and red. The Probe B oligo for each locus is highlighted in yellow. FIG. 33A shows the entire sequence and FIG. 33B to FIG. 33D each show one third of the sequence of FIG. 33A).

FIG. 34 shows results from the Multiplex SNV Experiment of Example 4. Three cell line DNA samples, SKMEL 2, SKMEL 5, and SKMEL 28 were genotyped for seven SNV mutations; gene and cosmic identifications are indicated in the plot. Signal over background is plotted for probes matching the wild type (WT) and mutant (Mut) sequences.

FIG. 35 shows the genotype determined by the Multiplex SNV Experiment of Example 4 compared to the genotype determined by other methods. qPCR results were determined using a TaqMan™ assay with samples taken from the same cell lines. NGS and WGS results are taken from literature.

FIG. 36 shows results from the 3D Biology experiments of Example 5, which simultaneously detect DNA SNV, RNA gene expression and Protein. Three cell lines were dosed with the BRAF inhibiting drug, vemurafenib. The three cell lines were SW 48 which is WT for the BRAF V600E mutation, RPMI 7591 which is heterozygous for the mutation, and SKMEL 28 which is a double mutant. Vemurafenib specifically targets cells containing the BRAF V600E mutation. All data was done in triplicate. The genotype of each cell line was determined using the SNV DNA Assay. Changes in gene expression (top) and protein expression (bottom) due to drug treatment are dependent on BRAF V600E genotype.

FIG. 37 shows three types of probes used for detecting proteins. In the top configuration, a probe comprises a nucleic acid attached to a protein-binding domain; in this configuration, a cleavable motif (e.g., a cleavable linker, not shown) may be included between the nucleic acid and protein-binding domain or within the nucleic acid itself. In the middle configuration, a protein-binding domain is attached to a nucleic acid and a probe hybridizes to the nucleic acid. The probe (comprising the target-binding domain and the nucleic acid attached to the protein-binding domain (shown in green)) can be bound by a probe before or after the target binding domain binds a protein target (As shown in FIG. 38). A cleavable motif may be included in either or both of the backbone or the nucleic acid attached to the protein-binding domain. In the bottom configuration, a protein-binding domain is attached to a nucleic acid and an intermediary oligonucleotide (shown in red) hybridizes to both a probe and to the nucleic acid attached to the protein-binding domain.

FIG. 38 shows the middle and bottom probes of FIG. 37. The top two images show the probe before and after it has bound a protein. The next image shows the probe after its cleavable motif has been cleaved; in this image the cleavable motif is between the nucleic acid and the target binding domain. Once the nucleic acid has been released, it can be considered a signal oligonucleotide. In the bottom image, the signal oligonucleotide (released nucleic acid of the probe) is bound by a reporter probe.

FIG. 39 shows release of signal oligonucleotides from a probe of the middle configuration shown in FIG. 37 and the probes of FIG. 38. The location of a cleavable motif within a probe (or in a reporter probe) affects which material is included with a released signal oligonucleotide.

FIG. 40 shows results from the KRAS Exon 2 Mutation HotSpot Experiment described in Example 6. Total counts in each hybridization reaction are dominated by reference counts and expected variant counts present at 5%.

FIG. 41 shows results from the EGFR Exon 19 Insertion-Deletion HotSpot Experiment described in Example 7. Total counts in each hybridization reaction are dominated by reference counts and expected variant counts for variant template present at 5%.

FIG. 42 shows results from the Multiplex SNV detection experiments in a Hotspot Experiment described in Example 8. Equal volume mixture of two FFPE-derived genomic DNA (gDNA) samples yields a sample with 10 mutations assayed by the SNV assay, with presence between 1-10%.

FIG. 43 shows comparison of variant probe counts from the Multiplex SNV detection experiments in a Hotspot Experiment described in Example 8.

FIG. 44 shows comparison of p-values from the Multiplex SNV detection experiments in a Hotspot Experiment described in Example 8. Significant differences in counts comparing reference sample to variant sample indicate the presence of mutant allele in the variant sample.

FIG. 45 shows the overall experimental workflow for simultaneous detection of SNVs and gene fusion transcripts as described in Example 9.

FIG. 46 shows a list of SNVs interrogated by SNV panel and features of the SNVs and primers used in the simultaneous SNV and fusion detection experiments as described in Example 9.

FIG. 47 shows SNV mutant allele-specific probe counts comparison for data described in Example 9. The comparison shows ˜100-fold more counts for the KRAS COSM532 mutation detected from the COSM532-containing FFPE gDNA sample versus the reference gDNA sample.

FIG. 48 shows SNV reference allele-specific probe count comparison for data described in Example 9. The comparison shows no significant differences in reference allele detection from the reference gDNA samples and the COSM532-containing FFPE gDNA sample.

FIG. 49 shows Lung Fusion Gene assay counts obtained while simultaneously assaying SNVs as described in Example 9. The data shows evidence of an EML4-ALK fusion only in the control sample.

FIG. 50 shows Lung Fusion Gene assay counts obtained while simultaneously assaying SNVs as described in Example 9. The data shows evidence of a CCDC6-RET fusion only in the control sample.

FIG. 51 shows Lung Fusion Gene assay counts obtained while simultaneously assaying SNVs as described in Example 9. The data shows evidence of an SLC34A2-ROS1 fusion only in the control sample.

FIG. 52 shows Lung Fusion Gene assay counts obtained while simultaneously assaying SNVs as described in Example 9. The data shows differing RNA sources used during simultaneous SNV and fusion transcript detection assays do not yield significantly different SNV assay probe counts.

### DETAILED DESCRIPTION OF THE INVENTION

The present invention is based in part on polymer strands, probes, compositions, methods, and kits for enabling accurate and robust enzyme- and amplification-free detection of DNA and RNA with single base resolution (e.g., detection of a single nucleotide polymorphism (SNP), an insertion, and a deletion).

The present invention can be combined with various ‘detection’ technologies (e.g., fluorescence, chromogenic, and mass spectrometry) for identifying and/or quantifying specific hybridization events. As examples, the polymer strands and partially double-stranded nucleic acid probes disclosed herein can be used with a wide variety of read-out reporters, for example, fluorescence (single molecule, multi-molecule via microparticles, DNA origami, rolling circle amplification, and branched DNA), chemiluminescence, chromogenic, mass tags (for mass spectrometry), and enzymatic. Thus, standard nucleic acid hybridization methods can be adapted for use with the probes of the present invention. Moreover, the polymer strands and partially double-stranded nucleic acid probes disclosed herein are compatible with and can be used with fluorescence optical barcode systems from NanoString Technologies®, e.g., the nCounter® systems; thus, there is minimal change in the nCounter® system's workflow. Together, the polymer strands and partially double-stranded nucleic acid probes disclosed herein provide multiplex nucleic acid detection and digital quantitation with single base resolution (e.g., detection of a single nucleotide polymorphism (SNP), an insertion, and a deletion); these are significant improvements upon currently-available technologies.

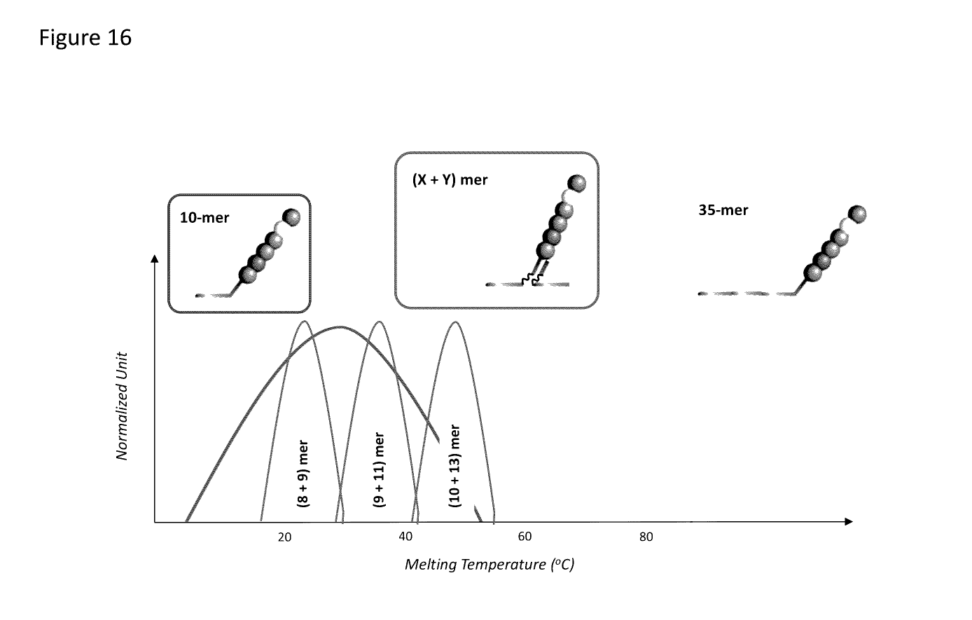

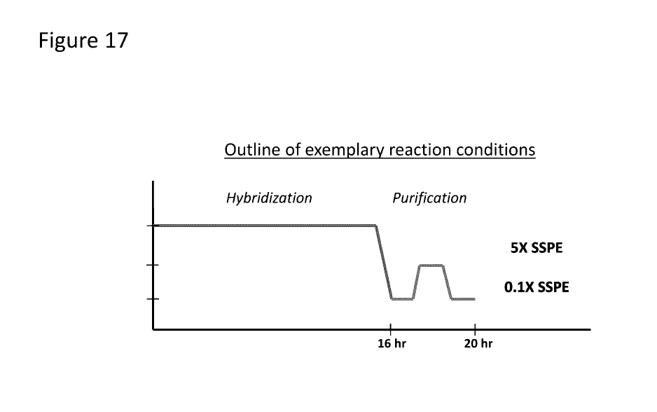

Designing probes for detecting a nucleic acid is a trade-off between stability and specificity. Longer probes (e.g., 35 base pairs) have high melting temps (Tm) and relatively narrow Tm distributions; these provide tight and complete binding but are less specific and are generally unable to detect single or two or more mismatches. On the other hand, shorter probes (e.g., 10 base pairs) have low Tm and very wide Tm distributions; these provide poor binding robustness but are very specific and allow for single base discrimination. The present invention solves this problem by disclosing probes having two short nucleic acid binding regions, which together provide stability and specificity and are able to detect single base substitutions. FIG. 16 illustrates these advantages of the present invention.

As shown in FIG. 16, short probes, having about 10 base pair target binding domains, have low temperature and widely distributed melting temperatures and long probes, having about 35 base pair target binding domains, have high temperature and narrowly distributed melting temperatures. In contrast, the present invention provides probes with two short target binding domains (e.g., 8 base pair plus 9 base pair; 9 base pair plus 11 base pair, and 10 base pair plus 13 base pair) that have low temperature and narrowly distributed melting temperatures.

In the present invention, the relatively short nucleic acid binding regions help maintain high specificity, enabling single base discrimination while two nucleic acid binding regions in tandem increase melting temperature of the hybridized arms when the sequence from both nucleic acid binding regions are a perfect match to the target sequence. A single mismatch in either nucleic acid binding region prevents stable hybridization due to the relatively short nucleic acid binding region lengths and one binding region alone is too short to maintain a stable hybridization. Only when both nucleic acid binding regions hybridize with a perfect match can a stable and specific hybridization be maintained and subsequently detected by various means.

The probes of the present invention undo the trade-off between stable, sensitive binding and sensitivity to a single base substitution previously required when designing probes for detecting nucleic acids.

Specific probes of the present invention are able to detect single base substitutions with greater than 99% accuracy. See, Example 1.

A first aspect of the present invention relates to a polymer strand pair including a first polymer strand having at least (1) a first target binding region, (2) a first complementary region, and (3) a sequence-specific region and a second polymer strand including at least (1) a second target binding region and (2) a second complementary region. The target of the first target binding region and the target of the second target binding region are in the same nucleic acid molecule and the target of the first target binding region is non-overlapping with the target of the second target binding region. The first complementary region is complementary to the second complementary region.

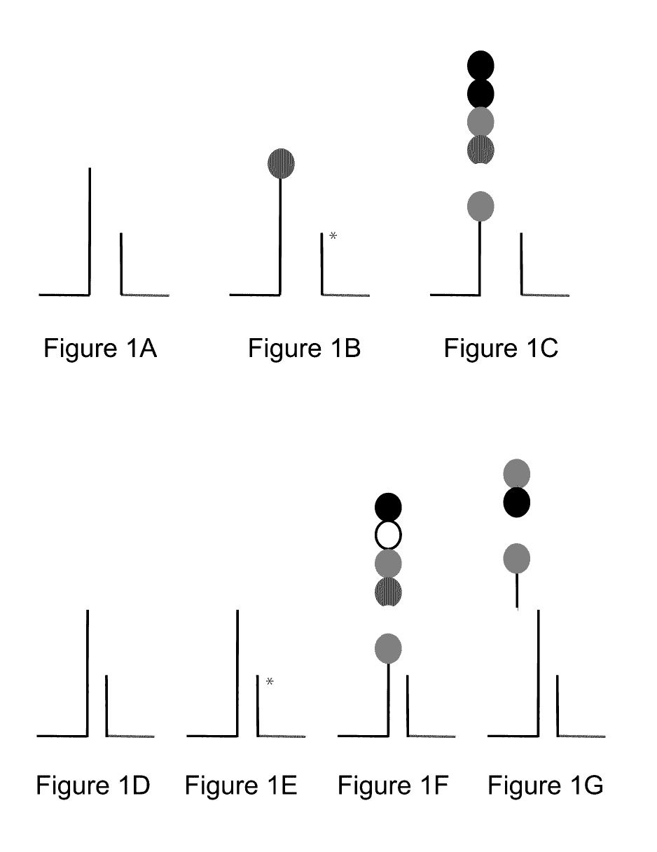

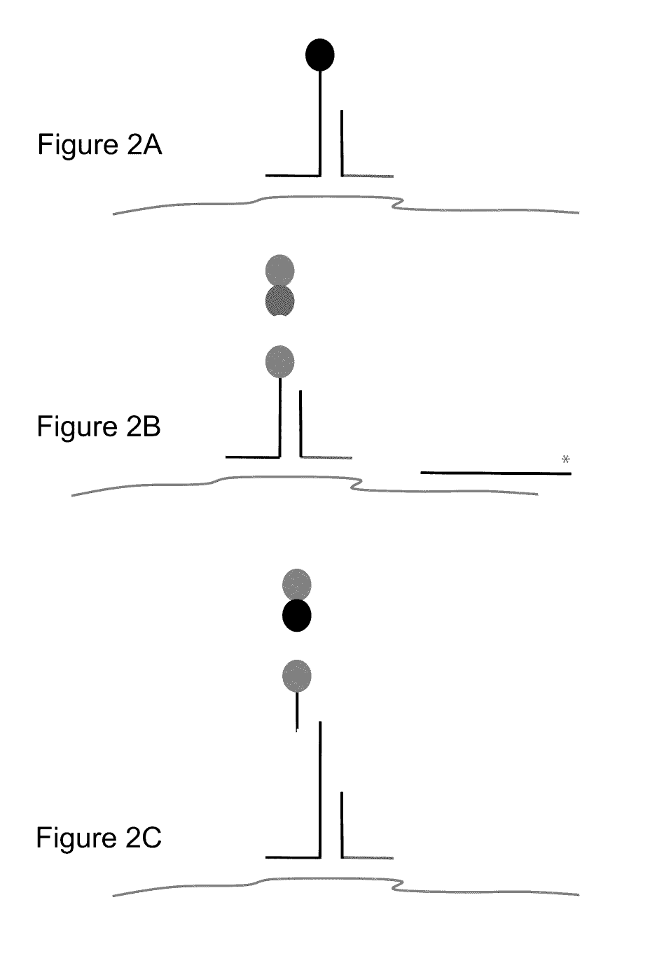

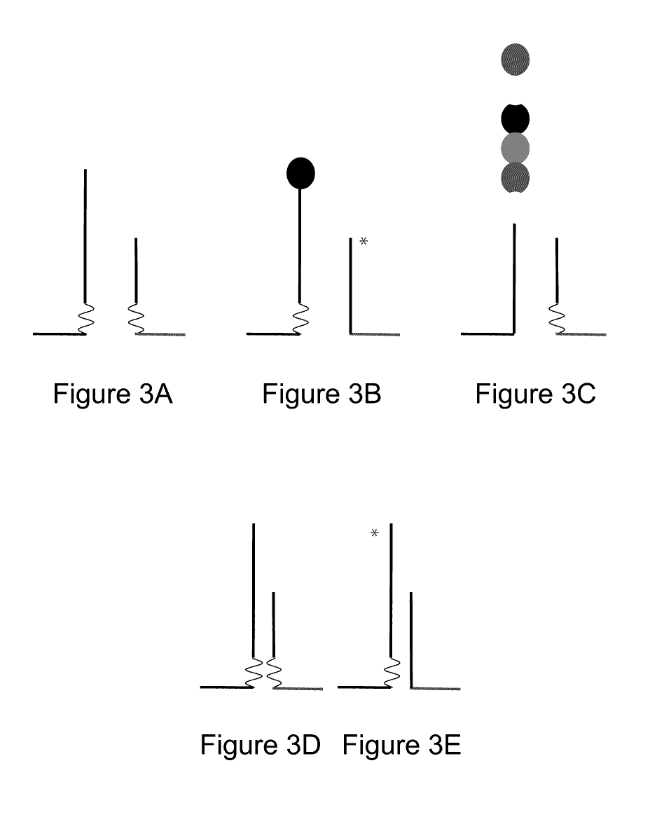

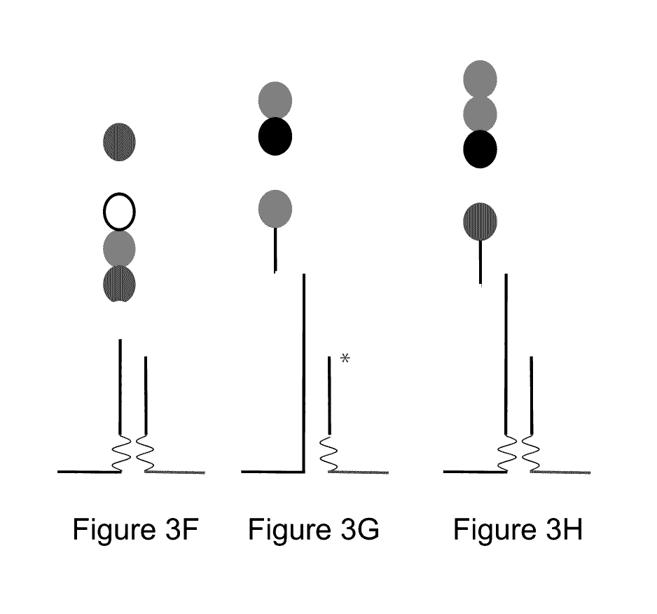

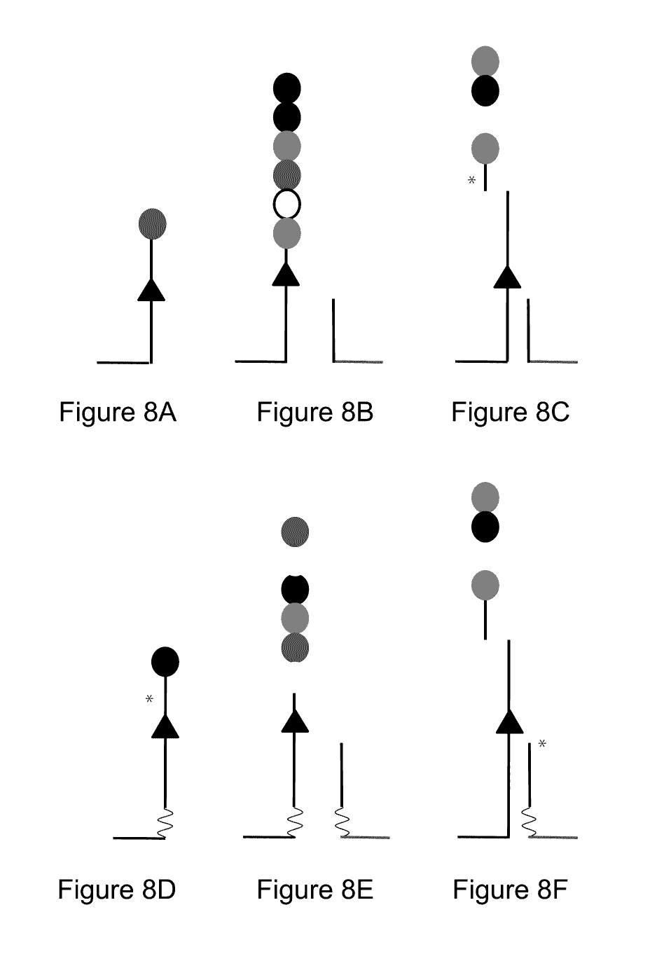

Exemplary polymer strand pairs of the first aspect are illustrated in FIGS. 1A to 1C, 3A to 3C, 8A, 8B, 8D, and 8E.

In embodiments of the first aspect, the first polymer strand may include a spacer (e.g., between the first target binding region and the first complementary region) and/or the second polymer strand may include a spacer (e.g., between the second target binding region and the second complementary region). The spacer may be polymer chain, e.g., an oligonucleotide and polyethylene glycol. The spacer between ‘folding joints’ relieves stress points for stabilization; a spacer may be included to alleviate ‘bending’ strain on the joint between regions or within a region.

In embodiments of the first aspect, at least one of the first target binding region, the first complementary region, and the sequence-specific region is a single stranded nucleic acid (e.g., DNA or RNA). The entire first polymer strand may be a single stranded nucleic acid molecule. At least one of the second target binding region and second complementary region is a single-stranded nucleic acid (e.g., DNA or RNA). The entire second polymer strand may be a single stranded nucleic acid molecule.

The first target binding region and second target binding region are capable of binding to a nucleic acid molecule (i.e., the same nucleic acid molecule). The nucleic acid molecule may be DNA, e.g., eukaryotic genomic DNA, mitochondrial DNA, chloroplast DNA, bacterial genomic DNA, archaebacterial genomic DNA, viral DNA, bacteriophage DNA, plasmid DNA, cDNA and synthetic (i.e., non-natural) DNA. The nucleic acid molecule may be RNA, e.g., messenger RNA (pre- or post-spliced mRNA), non-coding RNA (ncRNA), ribosomal RNA (rRNA), micro-RNA (miRNA), viral RNA, bacterial RNA, and synthetic (i.e., non-natural) RNA. The nucleic acid molecule may include at least one mutation relative to the corresponding wild-type nucleic acid molecule, e.g., a single nucleotide polymorphism (SNP), an insertion, a deletion, and a gene fusion. The at least one mutation may be in the target of the first target binding region and/or the target of the second target binding region; alternately or additionally, the mutation may be outside the two targets. In the case where the mutation corresponds to more than a single base change, one or more bases corresponding to the mutation may be in the target of the first target binding region and one or more of the bases corresponding to the mutation may be in the target of the second target binding region.

The target of the first target binding region and the target of the second target binding region may be separated by one or more nucleotides (e.g., about 1, 2, 3, 4, 5, 6, 7, 8, 9, 10, 11, 12, 13, 14, 15, 16, 17, 18, 19, 20, 30, 40, 50, 100, 1000, or more and any number in between); there is no upper limit to the separation distance between the two targets as long as the two polymer strands are capable of hybridizing to each other (via their complementary regions) and binding each target in the nucleic acid. In part, the length of one or both spacers determines the separation distance between the two targets, such that the longer the spacer or spacers, the further separated the two targets may be while still permitting stable binding to each target in the nucleic acid and stable hybridizing of the two polymer strands. Alternatively, the target of the first target binding region and the target of the second target binding region may be contiguous (i.e., not separated by a nucleotide).

The length of the first target binding region and the second target binding region are each between about 5 to about 35 nucleotides in length, e.g., 10 to 30 nucleotides. As examples, each target binding region may be about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides in length. The length of the first target binding region and the length of the second target binding region sum to no more than about 55 nucleotides (i.e., more than 10 nucleotides and less than 60 nucleotides and all sums in between).

The measured or predicted melting temperature of the first target binding region and/or the measured or predicted melting temperature of the second target binding region is between about 5° C. and about 35° C. (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and 35° C.). The measured or predicted melting temperature of the first target binding region and the measured or predicted melting temperature the second target binding region differ by about 30° C. or less (e.g., about 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0° C.). The first target binding region and the second target binding region may have about the same measured or predicted melting temperature. The measured or predicted melting temperature from the sum of the first target binding region and the second target binding region is between about 25° C. and about 60° C. (e.g., about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60° C.).

The first complementary region and/or the second complementary region may each be about 12 to about 60 nucleotides in length (e.g., about 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60).

In embodiments of the first aspect, the sequence-specific region of the first polymer strand may include at least two label attachment positions covalently linked in a linear combination. Each label attachment position is capable of binding at least one complementary single-stranded oligonucleotide, e.g., DNA or RNA. At least one single-stranded oligonucleotide includes at least one label monomer, e.g., biotin, chemiluminescent marker, dye, enzyme, fluorochrome, nanoparticle, quantum dot, and another monomer that can be detected directly or indirectly. An at least one label monomer at a first label attachment position is spectrally or spatially distinguishable from an at least one label monomer at an at least second label attachment position. A single-stranded oligonucleotide may lack a label monomer. The sequence-specific region may be attached to at least one affinity moiety, e.g., avidin, biotin, streptavidin or another moiety capable of being directly or indirectly captured upon a solid substrate.

In embodiments of the first aspect, the sequence-specific region of the first polymer strand is covalently attached to a single-stranded nucleic acid backbone, the backbone including at least two label attachment positions covalently linked in a linear combination. Each label attachment position is capable of binding at least one complementary single-stranded oligonucleotide, e.g., DNA or RNA. At least one single-stranded oligonucleotide includes at least one label monomer, e.g., biotin, chemiluminescent marker, dye, enzyme, fluorochrome, nanoparticle, quantum dot, and another monomer that can be detected directly or indirectly. An at least one label monomer at a first label attachment position is spectrally or spatially distinguishable from an at least one label monomer at an at least second label attachment position. A single-stranded oligonucleotide may lack a label monomer. The sequence-specific region and/or the single-stranded nucleic acid backbone may be attached to at least one affinity moiety, e.g., avidin, biotin, streptavidin or another moiety capable of being directly or indirectly captured upon a solid substrate.

In embodiments of the first aspect, the sequence-specific region is capable of binding to a portion of a reporter probe. The reporter probe includes at least a binding portion complementary to the sequence-specific region and a single-stranded nucleic acid backbone. The backbone including at least two label attachment positions covalently linked in a linear combination. Each label attachment position is capable of binding at least one complementary single-stranded oligonucleotide, e.g., DNA or RNA. At least one single-stranded oligonucleotide includes at least one label monomer, e.g., biotin, chemiluminescent marker, dye, enzyme, fluorochrome, nanoparticle, quantum dot, and another monomer that can be detected directly or indirectly. An at least one label monomer at a first label attachment position is spectrally or spatially distinguishable from an at least one label monomer at an at least second label attachment position. A single-stranded oligonucleotide may lack a label monomer. The reporter probe may be attached to at least one affinity moiety, e.g., avidin, biotin, streptavidin or another moiety capable of being directly or indirectly captured upon a solid substrate. The binding portion of the reporter probe that is complementary to the sequence-specific region is about 20 to about 50 nucleotides in length (e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50).

In embodiments of the first aspect, the sequence-specific region of the first polymer strand may include at least one label monomer, e.g., biotin, chemiluminescent marker, dye, enzyme, fluorochrome, nanoparticle, quantum dot, and another monomer that can be detected directly or indirectly. The at least one label monomer may be covalently attached to a nucleotide in the sequence-specific region or covalently attached to an oligonucleotide that is hybridized to a portion of the sequence-specific region. The sequence-specific region may be attached to at least one affinity moiety, e.g., avidin, biotin, streptavidin or another moiety capable of being directly or indirectly captured upon a solid substrate.

In embodiments of the first aspect, a second polymer strand may be attached to at least one affinity moiety, e.g., avidin, biotin, streptavidin or another moiety capable of being directly or indirectly captured upon a solid substrate.

In embodiments of the first aspect, the first polymer strand further comprises a cleavable linker between the first complementary region and the sequence-specific region; alternately, the cleavable linker is within the sequence-specific region. The cleavable linker may be photo-cleavable, chemically cleavable, and/or enzymatically cleavable. A photo-cleavable linker may be cleaved by light provided by a suitable coherent light source (e.g., a laser and a UV light source) or a suitable incoherent light source (e.g., an arc-lamp and a light-emitting diode (LED)).

A second aspect of the present invention relates to a method for detecting a nucleic acid in a sample including a step of contacting the sample with a polymer strand pair of the first aspect and/or of any embodiment of the first aspect. In this aspect, a first polymer strand has a sequence-specific region that (a) includes at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (b) is covalently attached to a single-stranded nucleic acid backbone, the backbone including at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (c) is bound or capable of being bound to a reporter probe, the reporter probe including at least a binding portion complementary to the sequence-specific region and a single-stranded nucleic acid backbone, the backbone including at least two label attachment positions covalently linked in a linear combination, with each label attachment position capable of binding at least one complementary single-stranded oligonucleotide including at least one label monomer, in which a linear combination of labelled monomers identifies the nucleic acid molecule; or (d) includes one or more label monomers, with the one or more label monomers identifying the nucleic acid molecule. The one or more label monomer is selected from biotin, chemiluminescent marker, dye, enzyme, fluorochrome, nanoparticle, quantum dot, and another monomer that can be detected directly or indirectly. This aspect includes a step of detecting the linear combination of labelled monomers or the one or more label monomers, thereby detecting the nucleic acid molecule in the sample.

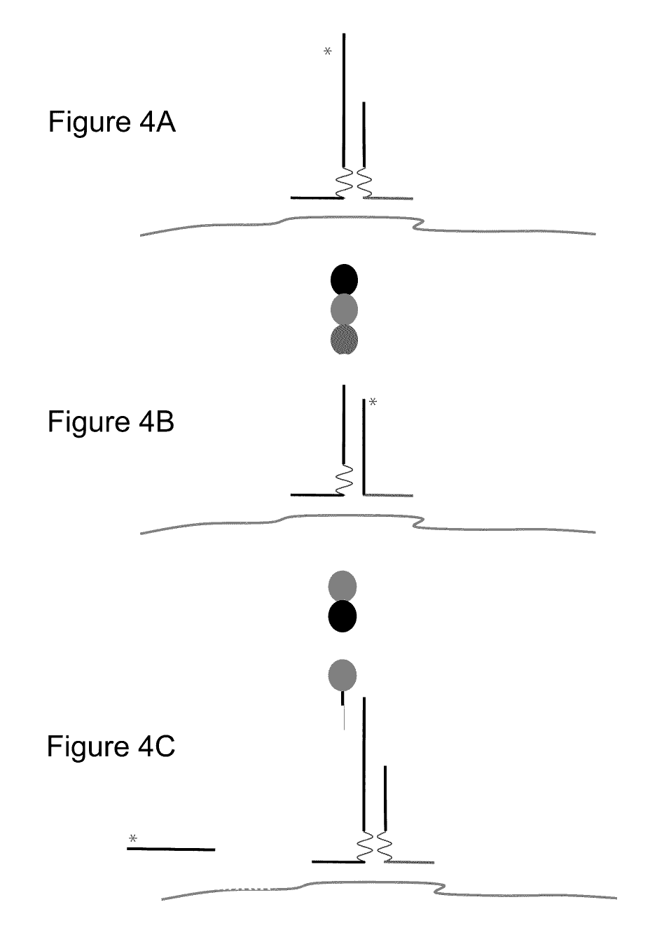

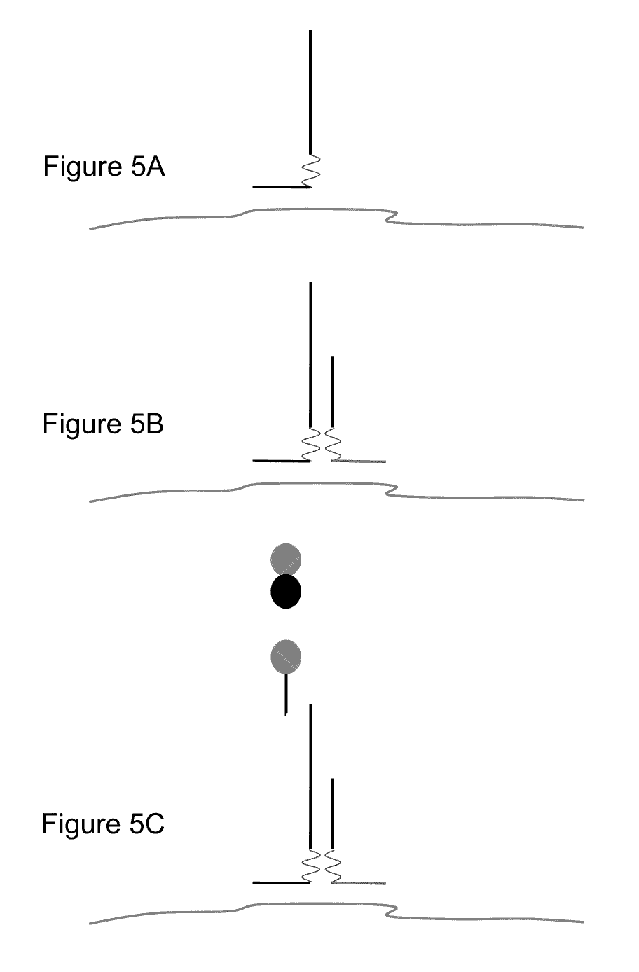

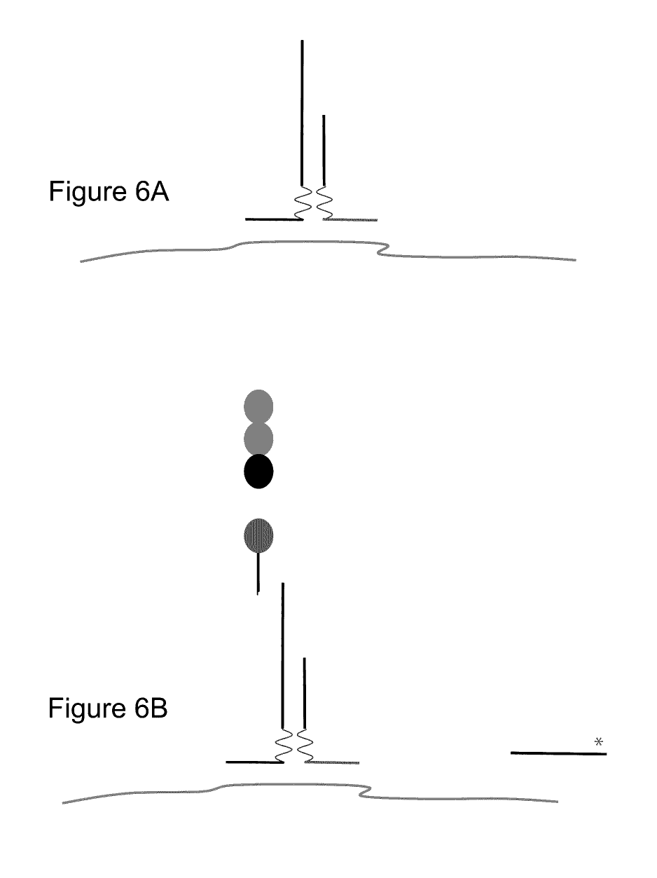

Exemplary methods for detecting a nucleic acid according to the second aspect are illustrated in FIGS. 2A, 2C, 4A, 4B, and 5.

In embodiments of the second aspect further includes contacting the sample with a capture polymer strand. The capture polymer strand includes at least (1) a region having at least one affinity moiety or a region capable of binding to a single-stranded nucleic acid including at least one affinity moiety and (2) a third target binding region capable of binding to the nucleic acid molecule. The targets of the first, second, and third target binding regions are non-overlapping and in the same nucleic acid molecule. The capture polymer strand is synonymous with a capture probe as described in the documents herein incorporated by reference.

A third aspect of the present invention relates to a composition including a plurality of polymer strand pairs of the first aspect and/or of any embodiment of the first aspect. In this aspect, a first polymer strand pair is capable of binding to a first nucleic acid molecule and an at least second polymer strand pair is capable of binding to an at least second nucleic acid molecule. The first nucleic acid molecule differs from the at least second nucleic acid molecule.

A fourth aspect of the present invention relates to a method for detecting a plurality of nucleic acids in a sample including a step of contacting the sample with a plurality of polymer strand pairs of the first aspect and/or of any embodiment of the first aspect or of contacting the sample with a composition of the third aspect and/or of any embodiment of the third aspect. In this aspect, each first polymer strand has a sequence-specific region that (a) includes at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (b) is covalently attached to a single-stranded nucleic acid backbone, the backbone including at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (c) is bound or capable of being bound to a reporter probe, the reporter probe including at least a binding portion complementary to the sequence-specific region and a single-stranded nucleic acid backbone, the backbone including at least two label attachment positions covalently linked in a linear combination, with each label attachment position capable of binding at least one complementary single-stranded oligonucleotide including at least one label monomer, in which a linear combination of labelled monomers identifies the nucleic acid molecule; or (d) includes one or more label monomers, with the one or more label monomers identifying the nucleic acid molecule. The one or more label monomer is selected from biotin, chemiluminescent marker, dye, enzyme, fluorochrome, nanoparticle, quantum dot, and another monomer that can be detected directly or indirectly. This aspect includes a step of detecting the linear combination of labelled monomers or the one or more label monomers for a first polymer strand pair and for an at least second polymer strand pair, thereby detecting the first nucleic acid molecule and the at least second nucleic acid molecule in the sample.

In embodiments of the fourth aspect further includes contacting the sample with a plurality of third polymers strands. The first capture polymer strand at least includes a region including at least one affinity moiety or including a region capable of binding to a single-stranded nucleic acid including at least one affinity moiety and a third target binding region that is capable of binding to a first nucleic acid molecule. Each at least second capture polymer strand at least includes a region including at least one affinity moiety or including a region capable of binding to a single-stranded nucleic acid comprising at least one affinity moiety and a third target binding region that is capable of binding to an at least second nucleic acid molecule. The targets of each first, second, and third target binding regions are non-overlapping and in the same nucleic acid molecule. The first nucleic acid molecule differs from the at least second nucleic acid molecule. A capture polymer strand is synonymous with a capture probe as described in the documents herein incorporated by reference.

A fifth aspect of the present invention relates to a polymer strand trio including a polymer strand pair of the first aspect and/or of any embodiment of the first aspect and a capture polymer strand. The capture polymer strand includes at least (1) a region having at least one affinity moiety or a region capable of binding to a single-stranded nucleic acid including at least one affinity moiety and (2) a third target binding region capable of binding to the nucleic acid molecule. The targets of the first, second, and third target binding regions are non-overlapping and in the same nucleic acid molecule. The capture polymer strand is synonymous with a capture probe as described in the documents herein incorporated by reference.

A sixth aspect of the present invention relates to a method for detecting a nucleic acid in a sample including a step of contacting the sample with a polymer strand trio of the fifth aspect and/or of any embodiment of the fifth aspect. In this aspect, a first polymer strand has a sequence-specific region that (a) includes at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (b) is covalently attached to a single-stranded nucleic acid backbone, the backbone including at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (c) is bound or capable of being bound to a reporter probe, the reporter probe including at least a binding portion complementary to the sequence-specific region and a single-stranded nucleic acid backbone, the backbone including at least two label attachment positions covalently linked in a linear combination, with each label attachment position capable of binding at least one complementary single-stranded oligonucleotide including at least one label monomer, in which a linear combination of labelled monomers identifies the nucleic acid molecule; or (d) includes one or more label monomers, with the one or more label monomers identifying the nucleic acid molecule. The one or more label monomer is selected from biotin, chemiluminescent marker, dye, enzyme, fluorochrome, nanoparticle, quantum dot, and another monomer that can be detected directly or indirectly. This aspect includes a step of detecting the linear combination of labelled monomers or the one or more label monomers on the polymer strand trio thereby detecting the nucleic acid molecule in the sample.

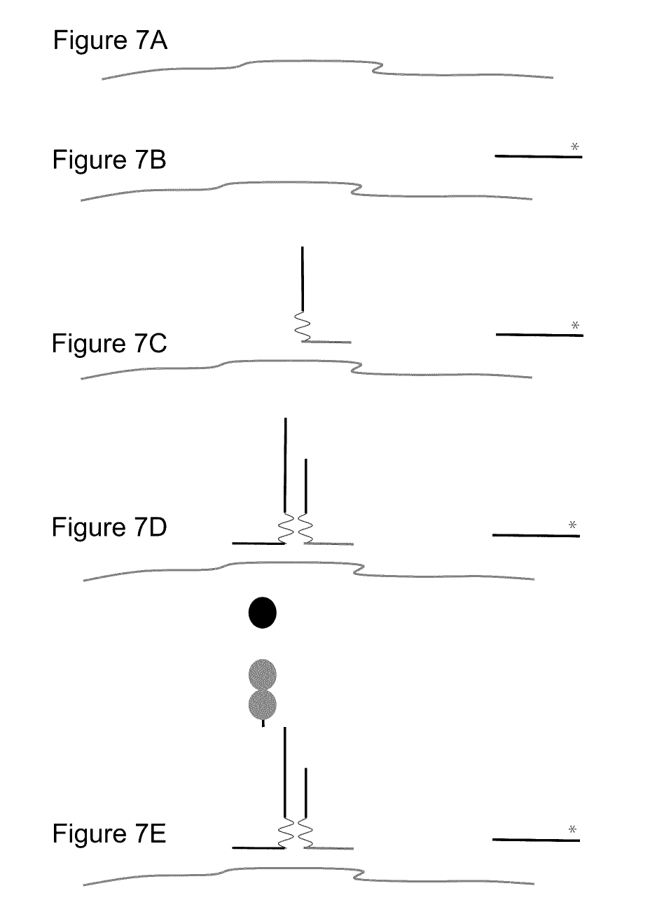

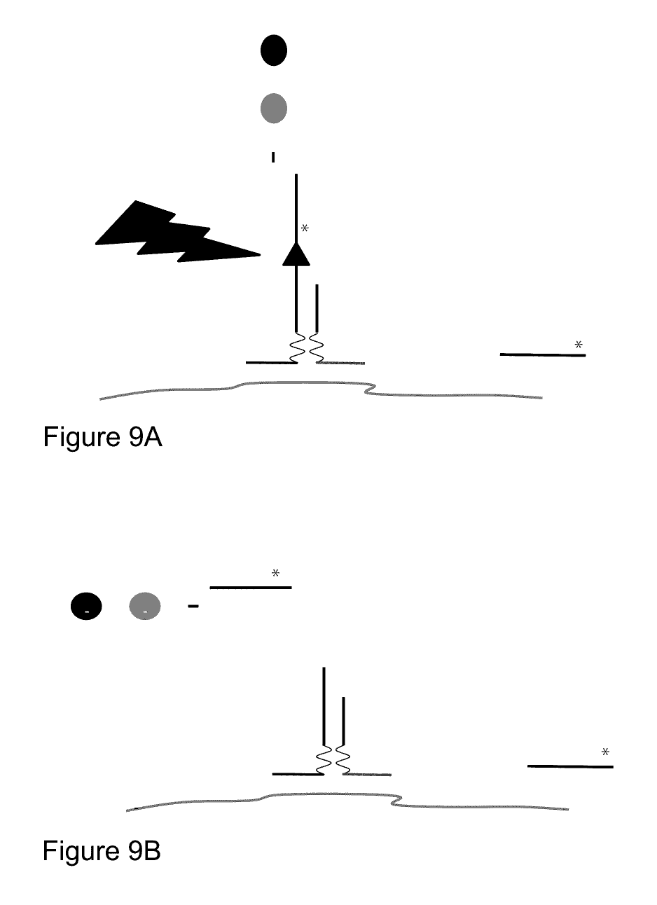

Exemplary methods for detecting a nucleic acid according to the sixth aspect are illustrated in FIGS. 2B, 4C, 6A, 6B, 7, and 9.

A seventh aspect of the present invention relates to a composition including a plurality of polymer strand trios of the fifth aspect and/or of any embodiment of the fifth aspect. In this aspect, a first polymer strand trio is capable of binding to a first nucleic acid molecule and an at least second polymer strand trio is capable of binding to an at least second nucleic acid molecule. The first nucleic acid molecule differs from the at least second nucleic acid molecule.

An eight aspect of the present invention relates to a method for detecting a plurality of nucleic acids in a sample including a step of contacting the sample with a plurality of polymer strand trios of the fifth aspect and/or of any embodiment of the fifth aspect or of contacting the sample with a composition of the seventh aspect and/or of any embodiment of the seventh aspect. In this aspect, each first polymer strand has a sequence-specific region that (a) includes at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (b) is covalently attached to a single-stranded nucleic acid backbone, the backbone including at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (c) is bound or capable of being bound to a reporter probe, the reporter probe including at least a binding portion complementary to the sequence-specific region and a single-stranded nucleic acid backbone, the backbone including at least two label attachment positions covalently linked in a linear combination, with each label attachment position capable of binding at least one complementary single-stranded oligonucleotide including at least one label monomer, in which a linear combination of labelled monomers identifies the nucleic acid molecule; or (d) includes one or more label monomers, with the one or more label monomers identifying the nucleic acid molecule. The one or more label monomer is selected from biotin, chemiluminescent marker, dye, enzyme, fluorochrome, nanoparticle, quantum dot, and another monomer that can be detected directly or indirectly. This aspect includes a step of detecting the linear combination of labelled monomers or the one or more label monomers for a first polymer strand trio and an at least second polymer strand trio, thereby detecting the first nucleic acid molecule and the at least second nucleic acid molecule in the sample.

A ninth aspect of the present invention relates to a partially double-stranded nucleic acid probe obtained when the first complementary region and the second complementary region of a polymer strand pair of the first aspect and/or of any embodiment of the first aspect are hybridized. Upon hybridization, the first polymer strand and the second polymer strand form a partially-double stranded nucleic acid probe having each feature of the polymer strand pair of the first aspect and/or of any embodiment of the first aspect. Additionally, partially double-stranded nucleic acid probe of this aspect has measured or predicted melting temperature from the first target and the second target of between about 40° C. and about 60° C. (e.g., about 40, 41, 42, 43, 44, 45, 46, 47, 48, 49, 50, 51, 52, 53, 54, 55, 56, 57, 58, 59, and 60° C.).

Exemplary partially-double stranded nucleic acid probes of the ninth aspect are illustrated in FIGS. 1D to 1G, 3D to 3G, 8C, and 8F.

A tenth aspect of the present invention relates to a method for detecting a nucleic acid in a sample including a step of contacting the sample with a partially double-stranded nucleic acid probe of the ninth aspect. In this aspect, a first polymer strand has a sequence-specific region that (a) includes at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (b) is covalently attached to a single-stranded nucleic acid backbone, the backbone including at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (c) is bound or capable of being bound to a reporter probe, the reporter probe including at least a binding portion complementary to the sequence-specific region and a single-stranded nucleic acid backbone, the backbone including at least two label attachment positions covalently linked in a linear combination, with each label attachment position capable of binding at least one complementary single-stranded oligonucleotide including at least one label monomer, in which a linear combination of labelled monomers identifies the nucleic acid molecule; or (d) includes one or more label monomers, with the one or more label monomers identifying the nucleic acid molecule. The one or more label monomer is selected from biotin, chemiluminescent marker, dye, enzyme, fluorochrome, nanoparticle, quantum dot, and another monomer that can be detected directly or indirectly. This aspect includes a step of detecting the linear combination of labelled monomers or the one or more label monomers, thereby detecting the nucleic acid molecule in the sample.

Exemplary methods for detecting a nucleic acid according to the tenth aspect are illustrated in FIGS. 2A to 2C, 4A, 4B, and 5.

In embodiments of the tenth aspect further includes contacting the sample with a capture polymer strand. The capture polymer strand includes at least (1) a region having at least one affinity moiety or a region capable of binding to a single-stranded nucleic acid including at least one affinity moiety and (2) a third target binding region capable of binding to the nucleic acid molecule. The targets of the first, second, and third target binding regions are non-overlapping and in the same nucleic acid molecule. The capture polymer strand is synonymous with a capture probe as described in the documents herein incorporated by reference.

An eleventh aspect of the present invention relates to a composition including a plurality of partially double-stranded nucleic acid probes of the ninth aspect and/or of any embodiment of the ninth aspect. In this aspect, a first double-stranded nucleic acid probes is capable of binding to a first nucleic acid molecule and an at least second double-stranded nucleic acid probe is capable of binding to an at least second nucleic acid molecule. The first nucleic acid molecule differs from the at least second nucleic acid molecule.

A twelfth aspect of the present invention relates to a method for detecting a plurality of nucleic acids in a sample including a step of contacting the sample with a plurality of partially double-stranded nucleic acid probes of the ninth aspect and/or of any embodiment of the ninth aspect or of contacting the sample with a composition of the eleventh aspect and/or of any embodiment of the eleventh aspect. In this aspect, each first polymer strand has a sequence-specific region that (a) includes at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (b) is covalently attached to a single-stranded nucleic acid backbone, the backbone including at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (c) is bound or capable of being bound to a reporter probe, the reporter probe including at least a binding portion complementary to the sequence-specific region and a single-stranded nucleic acid backbone, the backbone including at least two label attachment positions covalently linked in a linear combination, with each label attachment position capable of binding at least one complementary single-stranded oligonucleotide including at least one label monomer, in which a linear combination of labelled monomers identifies the nucleic acid molecule; or (d) includes one or more label monomers, with the one or more label monomers identifying the nucleic acid molecule. The one or more label monomer is selected from biotin, chemiluminescent marker, dye, enzyme, fluorochrome, nanoparticle, quantum dot, and another monomer that can be detected directly or indirectly. This aspect includes a step of detecting the linear combination of labelled monomers or the one or more label monomers for a first partially double-stranded nucleic acid probe and an at least second partially double-stranded nucleic acid probes, thereby detecting the first nucleic acid molecule and the at least second nucleic acid molecule in the sample.

In embodiments of the twelfth aspect further includes contacting the sample with a plurality of third polymers strands. The first capture polymer strand at least includes a region including at least one affinity moiety or including a region capable of binding to a single-stranded nucleic acid including at least one affinity moiety and a third target binding region that is capable of binding to a first nucleic acid molecule. Each at least second capture polymer strand at least includes a region including at least one affinity moiety or including a region capable of binding to a single-stranded nucleic acid comprising at least one affinity moiety and a third target binding region that is capable of binding to an at least second nucleic acid molecule. The targets of each first, second, and third target binding regions are non-overlapping and in the same nucleic acid molecule. A capture polymer strand is synonymous with a capture probe as described in the documents herein incorporated by reference.

A thirteenth aspect of the present invention relates to a composition including a plurality of partially double-stranded nucleic acid probes of the ninth aspect and/or of any embodiment of the ninth aspect and a plurality of capture polymer strands. A first capture polymer strand at least includes a region including at least one affinity moiety or including a region capable of binding to a single-stranded nucleic acid including at least one affinity moiety and a third target binding region that is capable of binding to a first nucleic acid molecule. Each at least second capture polymer strand at least includes a region including at least one affinity moiety or including a region capable of binding to a single-stranded nucleic acid comprising at least one affinity moiety and a third target binding region that is capable of binding to an at least second nucleic acid molecule. The targets of each first, second, and third target binding regions are non-overlapping and in the same nucleic acid molecule. The first nucleic acid molecule differs from the at least second nucleic acid molecule. A capture polymer strand is synonymous with a capture probe as described in the documents herein incorporated by reference.

A fourteenth aspect of the present invention relates to a multivalent polymer strand including at least (a) a first target binding region, (2) a second target binding region, (3) a spacer between the first target binding region and the second target binding region, and (4) a sequence-specific region. The target of the first target binding region and the target of the second target binding region are in the same nucleic acid molecule and the target of the first target binding region is non-overlapping with the target of the second target binding region. The spacer may be polymer chain, e.g., an oligonucleotide and polyethylene glycol. The spacer between ‘folding joints’ relieves stress points for stabilization; a spacer may be included to alleviate ‘bending’ strain on the joint between regions or within a region.

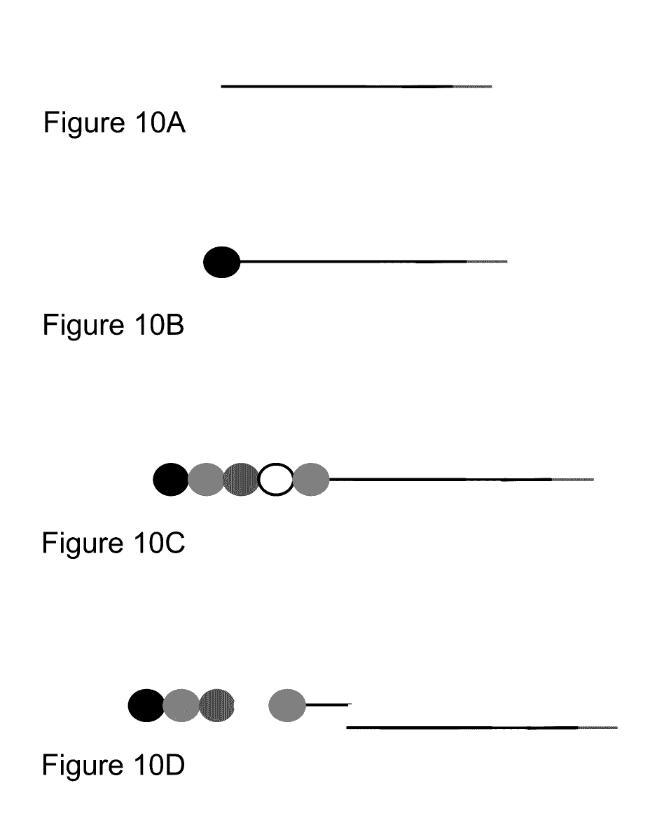

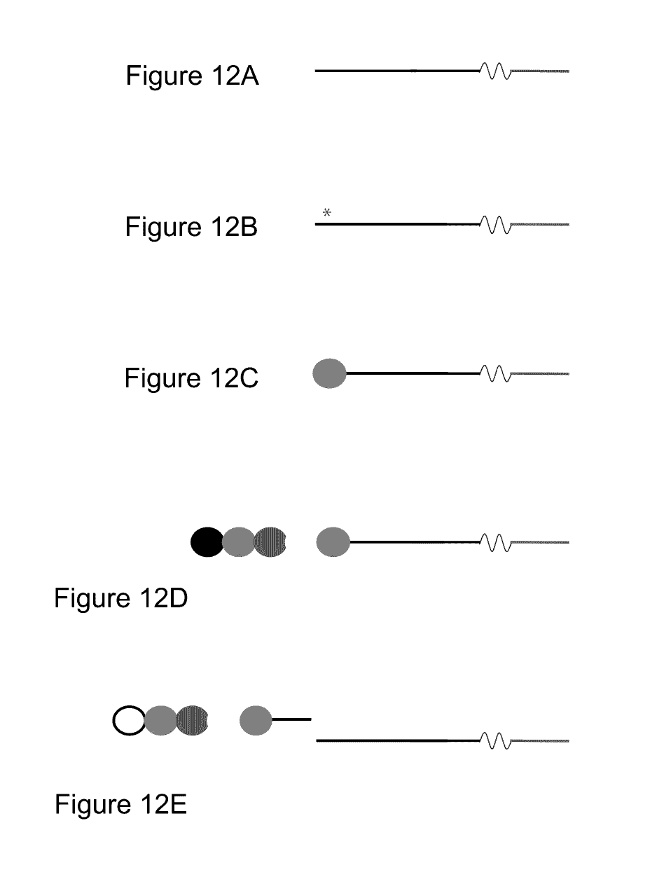

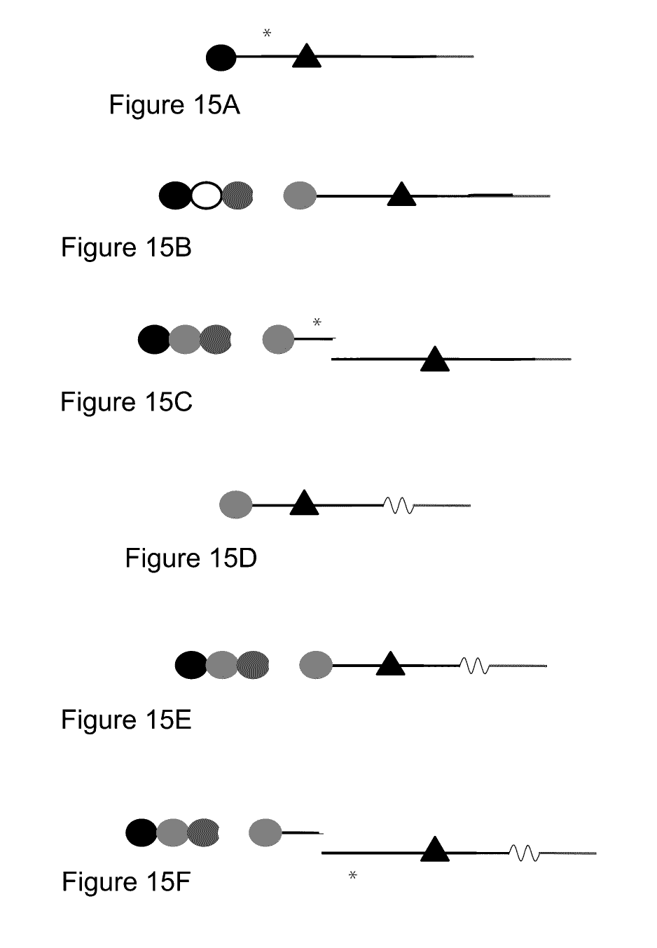

Exemplary multivalent polymer strands of the fourteenth aspect are illustrated in FIGS. 10, 12, and 15.

In embodiments of the fourteenth aspect, at least one of the first target binding region, the first, the second target binding region, and the sequence-specific region is a single stranded nucleic acid (e.g., DNA or RNA). The entire multivalent polymer strand may be a single stranded nucleic acid molecule.

The first target binding region and second target binding region are capable of binding to a nucleic acid molecule (i.e., the same nucleic acid molecule). The nucleic acid molecule may be DNA, e.g., eukaryotic genomic DNA, mitochondrial DNA, chloroplast DNA, bacterial genomic DNA, archaebacterial genomic DNA, viral DNA, bacteriophage DNA, plasmid DNA, cDNA and synthetic (i.e., non-natural) DNA. The nucleic acid molecule may be RNA, e.g., messenger RNA (pre- or post-spliced mRNA), non-coding RNA (ncRNA), ribosomal RNA (rRNA), micro-RNA (miRNA), viral RNA, bacterial RNA, and synthetic (i.e., non-natural) RNA. The nucleic acid molecule may include at least one mutation relative to the corresponding wild-type nucleic acid molecule, e.g., a single nucleotide polymorphism (SNP), an insertion, a deletion, and a gene fusion. The at least one mutation may be in the target of the first target binding region and/or the target of the second target binding region; alternately or additionally, the mutation may be outside the two targets.

The target of the first target binding region and the target of the second target binding region may be separated by one or more nucleotides (e.g., about 1, 2, 3, 4, 5, 6, 7, 8, 9, 10, 11, 12, 13, 14, 15, 16, 17, 18, 19, 20, 30, 40, 50, 100, 1000, or more and any number in between); there is no upper limit to the separation distance between the two targets as long as the two target binding regions are capable of binding to each target in the nucleic acid. In part, the length of the spacer determines the separation distance between the two targets, such that the longer the spacer or spacers, the further separated the two targets may be will still permitting stable binding to each target in the nucleic acid. The target of the first target binding region and the target of the second target binding region may be contiguous (i.e., not separated by a nucleotide).

The length of the first target binding region and the second target binding region are each between about 5 to about 35 nucleotides in length, e.g., 10 to 30 nucleotides. As examples, each target binding region may be about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 nucleotides in length. The length of the first target binding region and the length of the second target binding region sum to no more than about 55 nucleotides (i.e., more than 10 nucleotides and less than 60 nucleotides and all sums in between).

The measured or predicted melting temperature of the first target binding region and/or the measured or predicted melting temperature the second target binding region is between about 5° C. and about 35° C. (e.g., about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, and 35° C.). The measured or predicted melting temperature of the first target binding region and the measured or predicted melting temperature the second target binding region differ by about 30° C. or less (e.g., about 30, 29, 28, 27, 26, 25, 24, 23, 22, 21, 20, 19, 18, 17, 16, 15, 14, 13, 12, 11, 10, 9, 8, 7, 6, 5, 4, 3, 2, and 1° C.). The first target binding region and the second target binding region may have about the same measured or predicted melting temperature. The measured or predicted melting temperature from the sum of the first target binding region and the second target binding region is between about 25° C. and about 60° C. (e.g., about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, and 60° C.).

In embodiments of the fourteenth aspect, the sequence-specific region of the multivalent polymer strand may include at least two label attachment positions covalently linked in a linear combination. Each label attachment position is capable of binding at least one complementary single-stranded oligonucleotide, e.g., DNA or RNA. At least one single-stranded oligonucleotide includes at least one label monomer, e.g., biotin, chemiluminescent marker, dye, enzyme, fluorochrome, nanoparticle, quantum dot, and another monomer that can be detected directly or indirectly. An at least one label monomer at a first label attachment position is spectrally or spatially distinguishable from an at least one label monomer at an at least second label attachment position. A single-stranded oligonucleotide may lack a label monomer. The multivalent polymer strand may be attached to at least one affinity moiety, e.g., avidin, biotin, streptavidin or another moiety capable of being directly or indirectly captured upon a solid substrate.

In embodiments of the fourteenth aspect, the sequence-specific region of the multivalent polymer strand is covalently attached to a single-stranded nucleic acid backbone, the backbone including at least two label attachment positions covalently linked in a linear combination. Each label attachment position is capable of binding at least one complementary single-stranded oligonucleotide, e.g., DNA or RNA. At least one single-stranded oligonucleotide includes at least one label monomer, e.g., biotin, chemiluminescent marker, dye, enzyme, fluorochrome, nanoparticle, quantum dot, and another monomer that can be detected directly or indirectly. An at least one label monomer at a first label attachment position is spectrally or spatially distinguishable from an at least one label monomer at an at least second label attachment position. A single-stranded oligonucleotide may lack a label monomer. The sequence-specific region and/or the single-stranded nucleic acid backbone may be attached to at least one affinity moiety, e.g., avidin, biotin, streptavidin or another moiety capable of being directly or indirectly captured upon a solid substrate.

In embodiments of the fourteenth aspect, the sequence-specific region is capable of binding to a portion of a reporter probe. The reporter probe includes at least a binding portion complementary to the sequence-specific region and a single-stranded nucleic acid backbone. The backbone including at least two label attachment positions covalently linked in a linear combination. Each label attachment position is capable of binding at least one complementary single-stranded oligonucleotide, e.g., DNA or RNA. At least one single-stranded oligonucleotide includes at least one label monomer, e.g., biotin, chemiluminescent marker, dye, enzyme, fluorochrome, nanoparticle, quantum dot, and another monomer that can be detected directly or indirectly. An at least one label monomer at a first label attachment position is spectrally or spatially distinguishable from an at least one label monomer at an at least second label attachment position. A single-stranded oligonucleotide may lack a label monomer. The reporter probe may be attached to at least one affinity moiety, e.g., avidin, biotin, streptavidin or another moiety capable of being directly or indirectly captured upon a solid substrate. The binding portion of the reporter probe that is complementary to the sequence-specific region is about 20 to about 50 nucleotides in length (e.g., about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50).

In embodiments of the fourteenth aspect, the sequence-specific region of the multivalent polymer strand may include at least one label monomer, e.g., biotin, chemiluminescent marker, dye, enzyme, fluorochrome, nanoparticle, quantum dot, and another monomer that can be detected directly or indirectly. The at least one label monomer may be covalently attached to a nucleotide in the sequence-specific region or covalently attached to an oligonucleotide that is hybridized to a portion of the sequence-specific region. The sequence-specific region may be attached to at least one affinity moiety, e.g., avidin, biotin, streptavidin or another moiety capable of being directly or indirectly captured upon a solid substrate.

In embodiments of the fourteenth aspect, the multivalent polymer strand further comprises a cleavable linker between the second target binding and the sequence-specific region; alternately, the cleavable linker is within the sequence-specific region. The cleavable linker may be photo-cleavable, chemically cleavable, and/or enzymatically cleavable. A photo-cleavable linker may be cleaved by light provided by a suitable coherent light source (e.g., a laser and a UV light source) or a suitable incoherent light source (e.g., an arc-lamp and a light-emitting diode (LED)).

A fifteenth aspect of the present invention relates to a method for detecting a nucleic acid in a sample including a step of contacting the sample with a multivalent polymer strand of the fourteenth aspect and/or of any embodiment of the fourteenth aspect. In this aspect, the multivalent polymer strand has a sequence-specific region that (a) includes at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (b) is covalently attached to a single-stranded nucleic acid backbone, the backbone including at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (c) is bound or capable of being bound to a reporter probe, the reporter probe including at least a binding portion complementary to the sequence-specific region and a single-stranded nucleic acid backbone, the backbone including at least two label attachment positions covalently linked in a linear combination, with each label attachment position capable of binding at least one complementary single-stranded oligonucleotide including at least one label monomer, in which a linear combination of labelled monomers identifies the nucleic acid molecule; or (d) includes one or more label monomers, with the one or more label monomers identifying the nucleic acid molecule. The one or more label monomer is selected from biotin, chemiluminescent marker, dye, enzyme, fluorochrome, nanoparticle, quantum dot, and another monomer that can be detected directly or indirectly. This aspect includes a step of detecting the linear combination of labelled monomers or the one or more label monomers, thereby detecting the nucleic acid molecule in the sample.

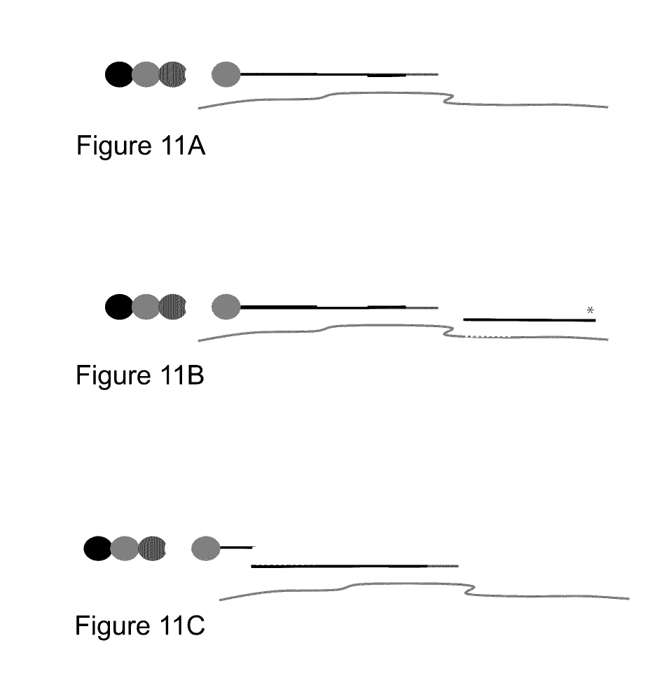

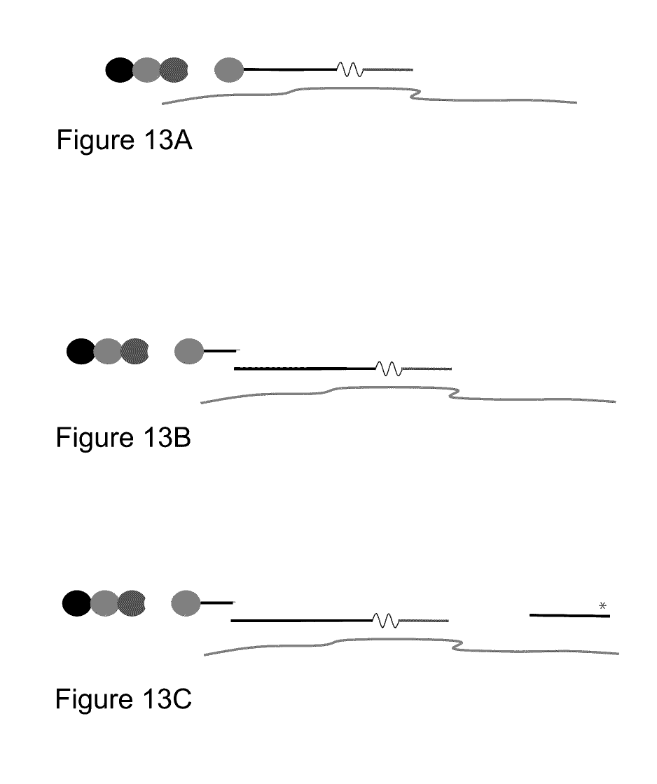

Exemplary methods for detecting a nucleic acid according to the fifteenth aspect are illustrated in FIGS. 11A, 11C, 13A, and 13B.

In embodiments of the fifteenth aspect further includes contacting the sample with a capture polymer strand. The capture polymer strand includes at least (1) a region having at least one affinity moiety or a region capable of binding to a single-stranded nucleic acid including at least one affinity moiety and (2) a third target binding region capable of binding to the nucleic acid molecule. The targets of the first, second, and third target binding regions are non-overlapping and in the same nucleic acid molecule. The capture polymer strand is synonymous with a capture probe as described in the documents herein incorporated by reference.

A sixteenth aspect of the present invention relates to a composition including a plurality of multivalent polymer strand of the fourteenth aspect and/or of any embodiment of the fourteenth aspect. In this aspect, a first multivalent polymer strand is capable of binding to a first nucleic acid molecule and an at least second multivalent polymer strand is capable of binding to an at least second nucleic acid molecule. The first nucleic acid molecule differs from the at least second nucleic acid molecule.

A seventeenth aspect of the present invention relates to a method for detecting a plurality of nucleic acids in a sample including a step of contacting the sample with a plurality of multivalent polymer strand of the fourteenth aspect and/or of any embodiment of the fourteenth aspect or of contacting the sample with a composition of the sixteenth aspect and/or of any embodiment of the sixteenth aspect. In this aspect, each multivalent polymer strand has a sequence-specific region that (a) includes at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (b) is covalently attached to a single-stranded nucleic acid backbone, the backbone including at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (c) is bound or capable of being bound to a reporter probe, the reporter probe including at least a binding portion complementary to the sequence-specific region and a single-stranded nucleic acid backbone, the backbone including at least two label attachment positions covalently linked in a linear combination, with each label attachment position capable of binding at least one complementary single-stranded oligonucleotide including at least one label monomer, in which a linear combination of labelled monomers identifies the nucleic acid molecule; or (d) includes one or more label monomers, with the one or more label monomers identifying the nucleic acid molecule. The one or more label monomer is selected from biotin, chemiluminescent marker, dye, enzyme, fluorochrome, nanoparticle, quantum dot, and another monomer that can be detected directly or indirectly. This aspect includes a step of detecting the linear combination of labelled monomers or the one or more label monomers for a first multivalent polymer strand and an at least second multivalent polymer strand, thereby detecting the first nucleic acid molecule and the at least second nucleic acid molecule in the sample.

In embodiments of the seventeenth aspect further includes contacting the sample with a plurality of capture polymers strands. The first capture polymer strand at least includes a region including at least one affinity moiety or including a region capable of binding to a single-stranded nucleic acid including at least one affinity moiety and a third target binding region that is capable of binding to a first nucleic acid molecule. Each at least second capture polymer strand at least includes a region including at least one affinity moiety or including a region capable of binding to a single-stranded nucleic acid comprising at least one affinity moiety and a third target binding region that is capable of binding to an at least second nucleic acid molecule. The targets of each first, second, and third target binding regions are non-overlapping and in the same nucleic acid molecule. The first nucleic acid molecule differs from the at least second nucleic acid molecule. A capture polymer strand is synonymous with a capture probe as described in those documents herein incorporated by reference.

An eighteenth aspect of the present invention relates to a multivalent polymer strand duo including a multivalent polymer strand of the fourteenth aspect and/or of any embodiment of the fourteenth aspect and a capture polymer strand. The capture polymer strand includes at least (1) a region having at least one affinity moiety or a region capable of binding to a single-stranded nucleic acid including at least one affinity moiety and (2) a third target binding region capable of binding to the nucleic acid molecule. The targets of the first, second, and third target binding regions are non-overlapping and in the same nucleic acid molecule. The capture polymer strand is synonymous with a capture probe as described in the documents herein incorporated by reference.

A nineteenth aspect of the present invention relates to a method for detecting a nucleic acid in a sample including a step of contacting the sample with a multivalent polymer strand duo of the eighteenth aspect and/or of any embodiment of the eighteenth aspect. In this aspect, a multivalent polymer strand has a sequence-specific region that (a) includes at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (b) is covalently attached to a single-stranded nucleic acid backbone, the backbone including at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (c) is bound or capable of being bound to a reporter probe, the reporter probe including at least a binding portion complementary to the sequence-specific region and a single-stranded nucleic acid backbone, the backbone including at least two label attachment positions covalently linked in a linear combination, with each label attachment position capable of binding at least one complementary single-stranded oligonucleotide including at least one label monomer, in which a linear combination of labelled monomers identifies the nucleic acid molecule; or (d) includes one or more label monomers, with the one or more label monomers identifying the nucleic acid molecule. The one or more label monomer is selected from biotin, chemiluminescent marker, dye, enzyme, fluorochrome, nanoparticle, quantum dot, and another monomer that can be detected directly or indirectly. This aspect includes a step of detecting the linear combination of labelled monomers or the one or more label monomers on the polymer strand trio, thereby detecting the nucleic acid molecule in the sample.

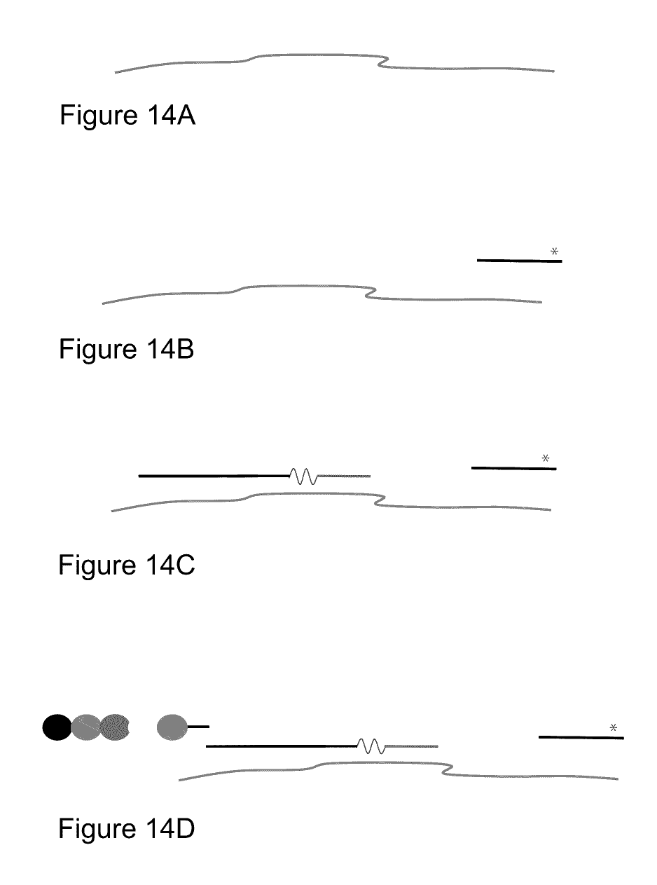

Exemplary methods for detecting a nucleic acid according to the nineteenth aspect are illustrated in FIGS. 11B, 13C, and 14.

A twentieth aspect of the present invention relates to a composition including a plurality of multivalent polymer strand duos of the eighteenth aspect and/or of any embodiment of the eighteenth aspect. In this aspect, a first multivalent polymer strand duo is capable of binding to a first nucleic acid molecule and an at least second multivalent polymer strand duo is capable of binding to an at least second nucleic acid molecule. The first nucleic acid molecule differs from the at least second nucleic acid molecule.

A twenty-first aspect of the present invention relates to a method for detecting a plurality of nucleic acids in a sample including a step of contacting the sample with a plurality of multivalent polymer strand duos of the eighteenth aspect and/or of any embodiment of the eighteenth aspect or of contacting the sample with a composition of the twentieth aspect and/or of any embodiment of the twentieth aspect. In this aspect, each multivalent polymer strand has a sequence-specific region that (a) includes at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (b) is covalently attached to a single-stranded nucleic acid backbone, the backbone including at least two label attachment positions covalently linked in a linear combination, with each label attachment position capable of binding at least one complementary single-stranded oligonucleotide including at least one label monomer, and in which a linear combination of labelled monomers identifies the nucleic acid molecule; (c) is bound or capable of being bound to a reporter probe, the reporter probe including at least a binding portion complementary to the sequence-specific region and a single-stranded nucleic acid backbone, the backbone including at least two label attachment positions covalently linked in a linear combination, with each label attachment position capable of binding at least one complementary single-stranded oligonucleotide including at least one label monomer, in which a linear combination of labelled monomers identifies the nucleic acid molecule; or (d) includes one or more label monomers, with the one or more label monomers identifying the nucleic acid molecule. The one or more label monomer is selected from biotin, chemiluminescent marker, dye, enzyme, fluorochrome, nanoparticle, quantum dot, and another monomer that can be detected directly or indirectly. This aspect includes a step of detecting the linear combination of labelled monomers or the one or more label monomers for a first polymer strand trio and an at least second polymer strand trio, thereby detecting the first nucleic acid molecule and the at least second nucleic acid molecule in the sample.

A twenty-second aspect of the present invention relates to a kit comprising a composition of the third aspect and/or of any embodiment of the third aspect, of the seventh aspect and/or of any embodiment of the seventh aspect, of the eleventh aspect and/or of any embodiment of the eleventh aspect, of the thirteenth aspect and/or of any embodiment of the thirteenth aspect, of the sixteenth aspect and/or of any embodiment of the sixteenth aspect, or of the twentieth aspect and/or of any embodiment of the twentieth aspect and instructions for use. The kit may further comprise at least one probe capable of detecting a protein target.

Any of the herein-described compositions may further comprise at least one probe capable of detecting a protein target.

Any of the herein-described methods may further comprise contacting a sample with at least one probe capable of detecting a protein target.

In each aspect of the present invention, a labeled oligonucleotide may be labeled with one or more detectable label monomers. The label may be at a terminus of an oligonucleotide, at a point within an oligonucleotide, or a combination thereof. Oligonucleotides may comprise nucleotides with amine-modifications, which allow coupling of a detectable label to the nucleotide. Labeled oligonucleotides of the present invention can be labeled with any of a variety of label monomers, such as a fluorochrome, quantum dot, dye, enzyme, nanoparticle, chemiluminescent marker, biotin, or other monomer known in the art that can be detected directly (e.g., by light emission) or indirectly (e.g., by binding of a fluorescently-labeled antibody). Preferred examples of a label that can be utilized by the invention are fluorophores. Several fluorophores can be used as label monomers for labeling nucleotides including, but not limited to GFP-related proteins, cyanine dyes, fluorescein, rhodamine, ALEXA Fluor™, Texas Red, FAM, JOE, TAN/IRA, and ROX. Several different fluorophores are known, and more continue to be produced, that span the entire spectrum.

In each aspect of the present invention, the number of attachment positions ranges from 1 to 100 or more. In embodiments, the number of positions ranges from 1, 2, 3, 4, 5, 6, 7, 8, 9, 10 to 15, 20, 30, 40, 50, 100 or any range in between. Indeed, the number of positions for detecting a nucleic acid is without limit since engineering such is well-within the ability of a skilled artisan. The number of nucleic acid molecules that are simultaneously detectable (“multiplexed”) depends on the number of label attachment positions. As examples, when two attachment positions are present and two possible color label monomers for each position, four nucleic acid molecules can be detected; when three attachment positions are present and two possible color label monomers for each position, eight nucleic acid molecules can be detected; and when three attachment positions are present and three possible color label monomers for each position, twenty-seven nucleic acid molecules can be detected.

In each aspect of the present invention, a label attachment position may be hybridized (non-covalently bound) with at least one labeled oligonucleotide. Alternately, a position may be hybridized with at least one oligonucleotide lacking a detectable label. Each position can hybridize to 1, 2, 3, 4, 5, 6, 7, 8, 9, 10, 11, 12, 13, 14, 15, 16, 17, 18, 19, 20, or 21 to 100 labeled (or unlabeled) oligonucleotides or more. The number of labeled oligonucleotides hybridized to each position depends on the length of the position and the size of the oligonucleotides. A position may be between about 300 to about 1500 nucleotides in length. The lengths of the labeled (or unlabeled) oligonucleotides vary from about 20 to about 1500 nucleotides in length. In embodiments, the lengths of labeled (or unlabeled) oligonucleotides vary from about 800 to about 1300 ribonucleotides. In other embodiments, the lengths of labeled (or unlabeled) oligonucleotides vary from about 20 to about 55 deoxyribonucleotides; such oligonucleotides are designed to have melting/hybridization temperatures of between about 65 and about 85° C., e.g., about 80° C. For example, a position of about 1100 nucleotides in length may hybridize to between about 25 and about 45 oligonucleotides comprising, each oligonucleotide about 45 to about 25 deoxyribonucleotides in length. In embodiments, each position is hybridized to about 34 labeled oligonucleotides of about 33 deoxyribonucleotides in length. The labeled oligonucleotides are preferably single-stranded DNA.

In each aspect of the present invention, labels associated with each position (via hybridization of a position with a labeled oligonucleotide) are spatially-separable and spectrally-resolvable from the labels of a preceding position or a subsequent position. An ordered series of spatially-separable and spectrally-resolvable labels of a probe is herein referred to as barcode or as a label code. The barcode or label code allows identification of a target nucleic acid or target protein that has been bound by a particular probe.

The terms “one or more”, “at least one”, and the like are understood to include but not be limited to at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between. Therefore, an “at least one complementary single-stranded oligonucleotide” may include, for example, 2 oligonucleotides, 6 oligonucleotides, and 10 oligonucleotides.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more and any number in between. Therefore, an “at least second polymer strand pair” includes, but is not limited to, 2 polymer strand pairs, 10 polymer strand pairs, 100 polymer strand pairs, and 1000 polymer strand pairs. Similarly, “at least second nucleic acid molecule” includes, but is not limited, to 2 nucleic acid molecules, 20 nucleic acid molecules, 40 nucleic acid molecules, and 60 nucleic acid molecules. Also, “at least second capture polymer strand” includes, but is not limited, to 2 capture polymer strands, 500 capture polymer strands, 1000 capture polymer strands, and 5000 capture polymer strands. Moreover, “at least two label attachment positions” includes, but is not limited, 2 label attachment positions, 4 label attachment positions, 6 label attachment positions, and 8 label attachment positions.

A polymer strand or probe may be chemically synthesized or may be produced biologically using a vector into which a nucleic acid encoding the probe has been cloned.

Any polymer strand or probe described herein may be used in methods and kits of the present invention.

As is well-known in the art, for a given nucleic acid, a measured or predicted melting temperature (Tm) value depends on the solution conditions, e.g., concentrations of monovalent (e.g., Na⁺) cations and divalent cations (e.g., Mg²⁺), and of free nucleotides (e.g., dNTPs). Also relevant is the concentration of polymer strands and target nucleic acid molecules. Generally, as recited in herein, Tms are “predicted” using algorithms that are known to those practiced in the art of nucleic acid chemistry; however, there is always a potential degree of error between measurement and prediction. This is typically plus or minus two to three degrees Celsius. Most commonly, the Tms recited herein are under standard solution concentrations for Tm prediction, as examples, 15 mM Na⁺, 0 mM 0 mM dNTPs, and 100 pM polymer strands and 820 mM Na⁺, 0 mM Mg²⁺, 0 mM dNTPs, and 250 nM polymer strands. It is also well-known that the length of a polymer strand and/or regions therein affects a measured or predicted melting temperature; thus, melting temperatures can be controlled by varying the length of polymer strand regions, e.g., target binding regions and/or complementary regions.

Any polymer strand or probe of the present invention may comprise an affinity moiety. Non-limiting examples of suitable affinity moieties are provided below. It should be understood that most affinity moieties could serve dual purposes: both as anchors for immobilization of a polymer strand, partially double-stranded probe, and/or reporter probe and moieties for purification of the same (whether fully or only partially assembled).

In certain embodiments, the affinity moiety is a protein monomer. Examples of protein monomers include, but are not limited to, the immunoglobulin constant regions (see, Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell. Bio. 4:220-229), the E. coli maltose binding protein (Guan et al., 1987, Gene 67:21-30), and various cellulose binding domains (U.S. Pat. Nos. 5,496,934; 5,202,247; and U.S. Pat. No. 5,137,819; Tomme et al., 1994, Protein Eng. 7:117-123). Other affinity moieties are recognized by specific binding partners and thus facilitate isolation and immobilization by affinity binding to the binding partner, which can be immobilized onto a solid support. For example, the affinity moiety can be an epitope, and the binding partner an antibody. Examples of such epitopes include, but are not limited to, the FLAG epitope, the myc epitope at amino acids 408-439, the influenza virus hemagglutinin (HA) epitope, or digoxigenin (“DIG”). In other embodiments, the affinity moiety is a protein or amino acid sequence that is recognized by another protein or amino acid, for example the avidin/streptavidin and biotin.

In embodiments, a polymer strand, partially double-stranded probe, and/or reporter probe can be immobilized to a substrate via an avidin-biotin binding pair. In certain embodiments, the polymer strand, partially double-stranded probe, and/or reporter probe comprises a biotin moiety and a substrate comprises avidin. Useful substrates comprising avidin are commercially available including TB0200 (Accelr8), SAD6, SAD20, SAD100, SAD500, SAD2000 (Xantec), SuperAvidin (Array-It®), streptavidin slide (catalog #MPC 000, Xenopore) and STREPTAVIDINnslide (catalog #439003, Greiner Bio-one). However, any substrate comprising avidin known to those of skill in the art may be used.

A substrate can take on any form so long as the form does not prevent selective immobilization of a polymer strand, partially double-stranded probe, and/or reporter probe comprising an affinity moiety. For instance, the substrate can have the form of a disk, slab, strip, bead, submicron particle, coated magnetic bead, gel pad, microtiter well, slide, membrane, frit or other form known to those of skill in the art. The substrate is optionally disposed within a housing, such as a chromatography column, spin column, syringe barrel, pipette, pipette tip, 96 or 384 well plate, microchannel, and capillary, that aids the flow of liquid over or through the substrate.

The present invention provides polymer strands, probes, methods, compositions, and kits for detecting one or more nucleic acids present in any sample, e.g., a biological sample. As will be appreciated by those in the art, the sample may comprise any number of things, including, but not limited to: cells (including both primary cells, cultured cell lines, dissociated cells from an explant), cell lysates or extracts (including but not limited to protein extracts, RNA extracts; purified mRNA), tissues (including cultured or explanted) and tissue extracts (including but not limited to protein extracts, RNA extracts; purified mRNA); bodily fluids (including, but not limited to, blood, urine, serum, lymph, bile, cerebrospinal fluid, interstitial fluid, aqueous or vitreous humor, colostrum, sputum, amniotic fluid, saliva, anal and vaginal secretions, perspiration and semen, a transudate, an exudate (e.g., fluid obtained from an abscess or any other site of infection or inflammation) or fluid obtained from a joint (e.g., a normal joint or a joint affected by disease such as rheumatoid arthritis, osteoarthritis, gout or septic arthritis) of virtually any organism, with mammalian samples being preferred and human samples being particularly preferred; environmental samples (including, but not limited to, air, agricultural, water and soil samples); biological warfare agent samples; research samples including extracellular fluids, extracellular supernatants from cell cultures, inclusion bodies in bacteria, cellular compartments, cellular periplasm, and mitochondria compartment.

The sample can be obtained from virtually any organism including multicellular organisms, e.g., of the plant, fungus, and animal kingdoms; preferably, the sample is obtained from an animal, e.g., a mammal. Human samples are particularly preferred.

The biological samples may be indirectly derived from biological specimens. For example, where the target nucleic acid is a cellular transcript, e.g., an mRNA, the biological sample of the invention can be a sample containing cDNA produced by a reverse transcription of mRNA. In another example, the biological sample of the invention is generated by subjecting a biological specimen to fractionation, e.g., size fractionation or membrane fractionation.

The sample may be cells (live or fixed) or tissue sections (live or fixed, e.g., formalin-fixed paraffin embedded (FFPE)) that are prepared consistent with nucleic acid in situ hybridization methods or immunohistochemistry methods are prepared and immobilized onto a glass slide or suitable solid support. The tissue sample may be embedded, serially sectioned, and immobilized onto a microscope slide. Access to the surface of cells or tissue-section is preserved, thereby allowing for fluidic exchange; this can be achieved by using a fluidic chamber reagent exchange system (e.g., Grace™ Bio-Labs, Bend Oreg.). Serial tissue sections may be approximately 5 μm to 15 μm from each other. Blocking steps may be performed before and/or after polymer strands, probes, or compositions are applied.

In some embodiments, the polymer strands, probes, compositions, methods, and kits described herein are used in the diagnosis of a condition. As used herein the term diagnose or diagnosis of a condition includes predicting or diagnosing the condition, determining predisposition to the condition, monitoring treatment of the condition, diagnosing a therapeutic response of the disease, and prognosis of the condition, condition progression, and response to particular treatment of the condition. For example, a tissue sample can be assayed according to any of the polymer strands, partially double-stranded nucleic acid probes, methods, or kits described herein to determine the presence and/or quantity of markers of a disease or malignant cell type in the sample (relative to the non-diseased condition), thereby diagnosing or staging a disease or a cancer. The tissue sample may be a biopsied tumor or a portion thereof, i.e., a clinically-relevant tissue sample. For example, the tumor may be from a breast cancer. The sample may be an excised lymph node. The tissue sample may be a liquid biopsy which may contain a tumor cell (e.g., from a solid tumor or a liquid tumor), a nucleic acid released from the tumor cell, or a protein released from the tumor cell.

Compositions and kits of the present invention can include polymer strands, partially double-stranded nucleic acid probes, and other reagents, for example, buffers and other reagents known in the art to facilitate binding of a protein and/or a nucleic acid in a sample, i.e., for performing hybridization reactions.

A kit also will include instructions for using the components of the kit, including, but not limited to, information necessary to hybridize labeled oligonucleotides to a polymer strand or a reporter probe, to bind a reporter probe to a polymer strand or to a partially double-stranded nucleic acid probe, to bind a polymer strand or partially double-stranded nucleic acid probes to a nucleic acid molecule in a sample, to hybridize a first polymer strand and a second polymer strand to form a partially double-stranded nucleic acid probe.

A kit can further include an apparatus which includes a surface suitable for binding, and optionally detecting polymer strands or partially double-stranded nucleic acid probes, and/or reporter probes included with the kit. The surface may be bound by any means known in the art.

The kit can further include a composition for the extraction of a nucleic acid from a biological sample.

A kit can further include a reagent selected from the group consisting of a hybridization reagent, a purification reagent, an immobilization reagent, and an imaging reagent.

Polymer strands comprising labelled monomers and/or reporter robes can be detected and quantified using commercially-available cartridges, software, systems, e.g., the nCounter® System using the nCounter® Cartridge.

The basis of the nCounter® Analysis system is the unique code assigned to each nucleic acid molecule to be assayed (International Patent Application No. PCT/US2008/059959 and Geiss et al. Nature Biotechnology. 2008. 26(3): 317-325; the contents of which are each incorporated herein by reference in their entireties). The code is composed of an ordered series of colored fluorescent spots which create a unique barcode for each nucleic acid molecule to be assayed.

Generally, a herein-described method may use DV2 reagents from NanoString Technologies®. With DV2 reagents, a first polymer strand and/or partially double-stranded probe is covalently attached to a single-stranded nucleic acid backbone, the backbone including at least two label attachment positions covalently linked in a linear combination, with each label attachment position capable of binding at least one complementary single-stranded oligonucleotide including at least one label monomer, and in which a linear combination of labelled monomers identifies the nucleic acid molecule. Additionally, if a capture polymer strand or capture probe is used, it includes, at least (1) a region capable of binding to a single-stranded nucleic acid including at least one affinity moiety and (2) a third target binding region capable of binding to the nucleic acid molecule.

A herein-described method using DV2 reagents from NanoString Technologies® includes, at least, the following steps: (1) Design polymer strands/probes covering a portion of a nucleic acid (e.g., where a single nucleotide polymorphism (SNP), an insertion, a deletion, and a gene fusion is located) and according to a set of design rules that achieve maximum design reliability/robustness; (2) mix all necessary components of DV2 reagents at appropriate concentrations along with nucleic acid molecule and incubate at defined temperature(s) and time(s) for hybridization; and (3) perform necessary purification to remove excess polymer strand/probes, capture polymer strands, and/or reporter probes prior to fluorescence detection and analysis. Each of these steps has been more fully described in the patent literature published by NanoString Technologies®; see, e.g., US2014/0371088.

In general, the DV2 system from NanoString Technologies® pertains to a multiplexable tag-based reporter system and methods for production and use. The tag-based nanoreporter system allows economical and rapid flexibility in the assay design, as the gene-specific components of the assay are separated from the reporter probe and capture reagents and are enabled by inexpensive and widely available DNA oligonucleotides. A single set of reporter probes can be used as readout for an infinite variety of genes in different experiments simply by replacing the gene-specific oligonucleotide (i.e., a polymer strand including a target binding region) portion of the assay. In the non-tag-based reporter system, the reporter probe and capture reagents (e.g., the label attachment regions and attached labels, and affinity moieties) are covalently attached (directly or indirectly) to the target binding regions. These are complicated and costly to modify.

nCounter® probes, systems, and methods from NanoString Technologies®, as described in US2003/0013091, US2007/0166708, US2010/0015607, US2010/0261026, US2010/0262374, US2010/0112710, US2010/0047924, US2014/0371088, US2014/0017688, and US2011/0086774), are a preferred means for identifying target proteins and/or target nucleic acids. nCounter® probes, systems, and methods from NanoString Technologies® allow simultaneous multiplexed identification a plurality (800 or more) distinct target proteins and/or target nucleic acids. Each of the above-mentioned patent publications is incorporated herein by reference in its entirety. The above-mentioned nCounter® probes, systems, and methods from NanoString Technologies® can be combined with any aspect or embodiment described herein.

A single nCounter® cartridge (e.g., a single lane thereof) may be used for simultaneous multiplexed identification of a plurality distinct target proteins and/or target nucleic acids from the combination of the above-mentioned nCounter® probes, systems, and methods and the aspects or embodiments described herein.

The relative abundance of each nucleic acid molecule in a plurality of nucleic acid molecules in a sample may be measured in a single multiplexed hybridization reaction. A sample is combined with a plurality of polymer strands, multivalent polymer strands, partially-double stranded nucleic acid probes, compositions and for forth, and hybridization occurs. Label monomers are detected using a fully automated imaging and data collection device (Digital Analyzer, NanoString Technologies®), thereby the abundance of each nucleic acid nucleic acid molecule is quantified. For each sample, ˜600 fields-of-view (FOV) are imaged (1376×1024 pixels) representing approximately 10 mm² of a binding surface. Typical imaging density is ˜100-1200 counted reporter probes per field of view depending on the degree of multiplexing, the amount of sample input, and overall nucleic acid molecule abundance. Data is output in simple spreadsheet format listing the number of counts per nucleic acid molecule, per sample.

Label monomers of the present invention can be detected by any means available in the art that is capable of detecting the specific signals. Where the label monomer fluoresces, suitable consideration of appropriate excitation sources may be investigated. Possible sources may include but are not limited to arc lamp, xenon lamp, lasers, light emitting diodes or some combination thereof. The appropriate excitation source is used in conjunction with an appropriate optical detection system, for example an inverted fluorescent microscope, an epi-fluorescent microscope or a confocal microscope. Preferably, a microscope is used that can allow for detection with enough spatial resolution to determine the sequence of the spots on the on a polymer strand or reporter probe

The single nucleotide variation (SNV) probes and methods disclosed herein permit detection of somatic variants with about 5% allele frequency from as little as 5 ng fresh or formalin-fixed paraffin embedded (FFPE) genomic DNA (gDNA).

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless defined otherwise, all technical and scientific terms used herein have the same meaning as commonly understood by one of ordinary skill in the art to which the invention pertains. Although other polymer strands, probes, compositions, methods, and kits similar, or equivalent, to those described herein can be used in the practice of the present invention, the preferred materials and methods are described herein. It is to be understood that the terminology used herein is for the purpose of describing particular embodiments only, and is not intended to be limiting.

### EXAMPLES

### Example 1

### SNP Detection Experiments Using Polymer Strand Pairs/Partially Double-Stranded Probes with Existing DV2 Reporter Probes from NanoString Technologies®

Experiments detecting the BRAF gene's V600E single nucleotide polymorphism (SNP) were performed.

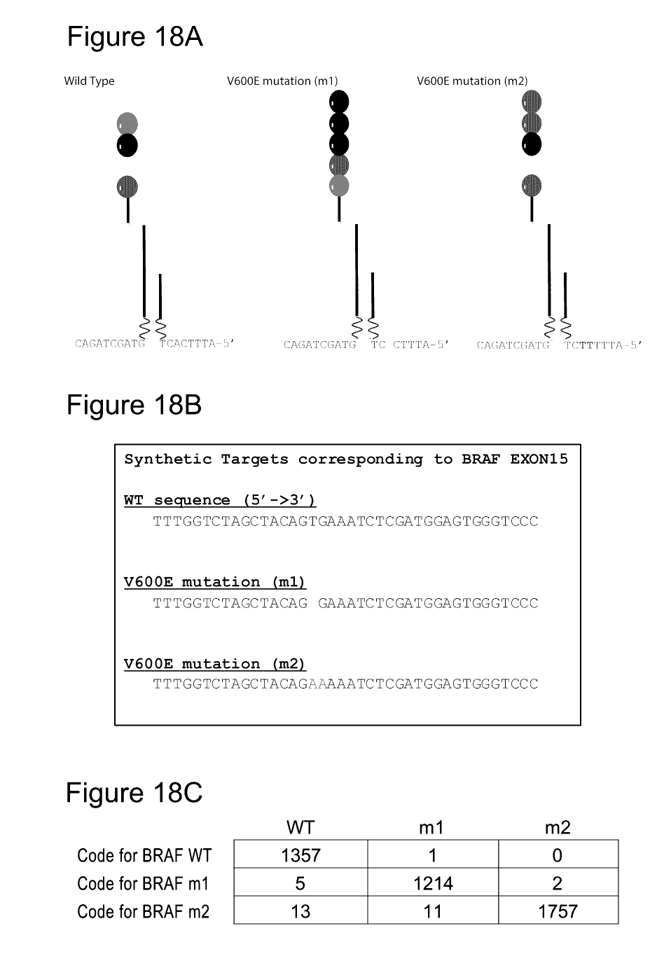

FIG. 18A shows three partially double-stranded probes used in this Example: a first probe for detecting the wild-type target and two probes (m1 and m2) for detecting two mutant versions of the target. In this experiment, the NanoString Technologies® DV2 system was used. The probes included target binding regions that were 10 nucleotides and 8 nucleotides in length (“10+8”), with the m1 and m2 mutation detected by the 8 nucleotide target binding region.

Synthetic targets (corresponding to BRAF Exon 15) for the three probes are shown in FIG. 18B. In this experiment, the target of a first target binding domain and the target of a second target binding domain are contiguous.

Hybridization was performed at room temperature. No purification step was performed prior to imaging on NanoString Technologies nCounter® Digital Analyzer with 25 fields-of-view (FOV).

Excellent results, with 99% accurate single base discrimination, were observed with the 10+8 probe. See, FIG. 18C.

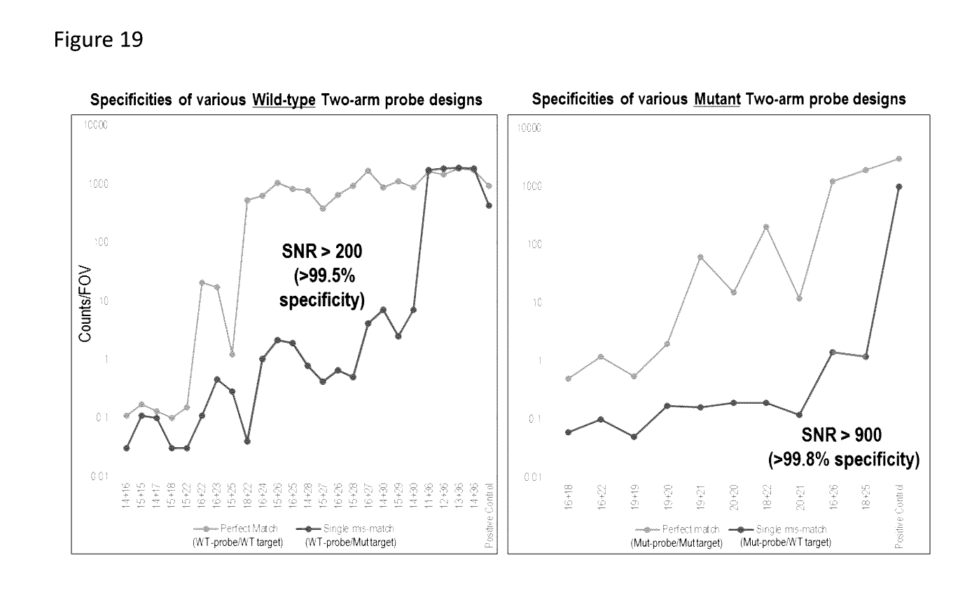

Probes with other length target binding domain (i.e., 11+36, 12+36, 13+36, 14+16, 14+17, 14+28, 14+30, 14+36, 15+15, 15+18, 15+22, 15+25, 15+26, 15+27, 15+28, 15+29, 16+18, 16+22, 16+23, 16+24, 16+25, 16+26, 16+27, 18+22, 18+25, 19+19, 19+20, 19+21, 20+20, and 20+21) where tested. In particular, there was >99.5% specificity for wild-type allele detection for eleven of the probes (see, the purple band of FIG. 19, left panel). There also was >99.8% specificity for mutant allele detection for two of the probes (see, the purple band of FIG. 19, right panel).

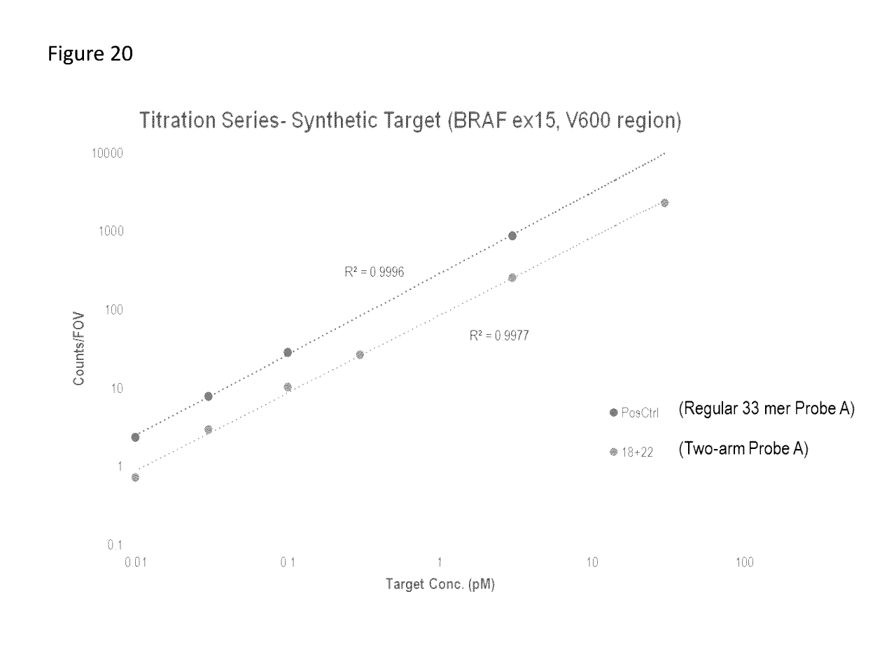

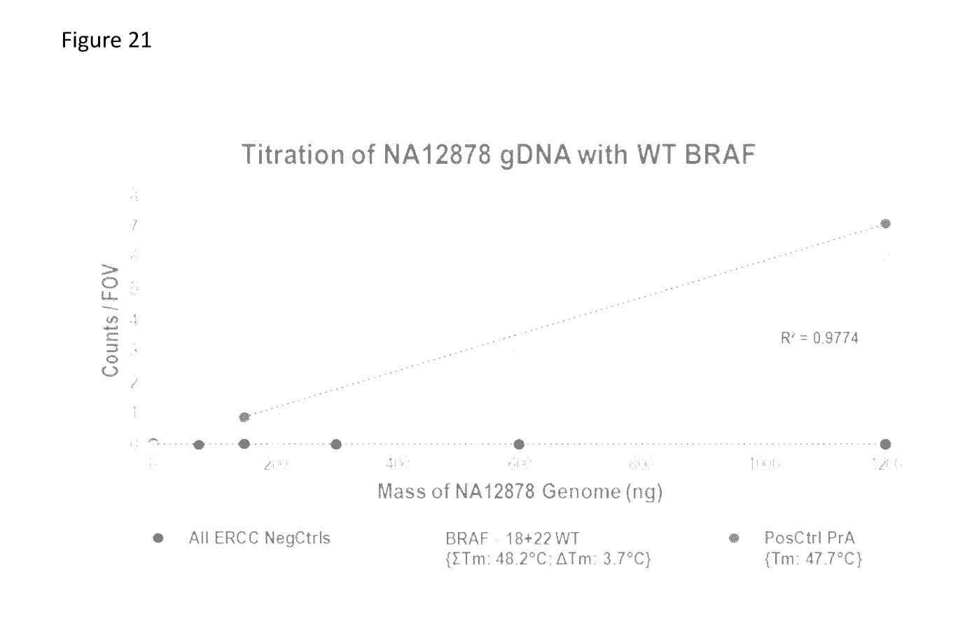

Additionally, the probes of the present invention were shown to be highly sensitive and capable of detecting the wild-type target in synthetic nucleic acids down to about 10 fM (see, FIG. 20) and capable of detecting the wild-type target in an about 75 ng sample of genomic DNA (see, FIG. 21).

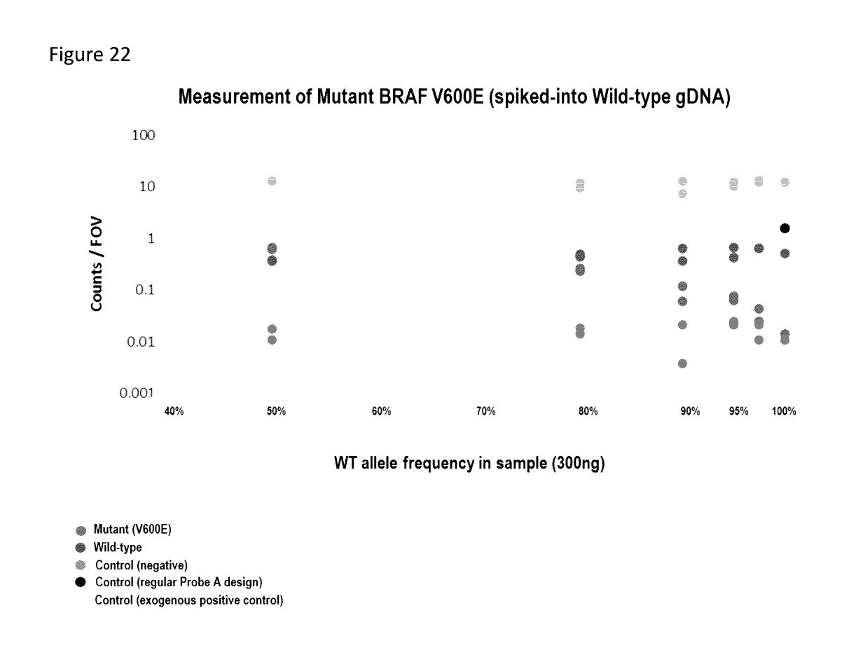

The probes of the present invention were shown to be capable of detecting a synthetic mutant target nucleic acid that had been mixed with wild-type genomic DNA. Here, probes were able to detect a solution comprising ˜5% of the synthetic mutant target nucleic acid in 300 ng of total genomic DNA sample (background levels determined via negative control measurements). (see, FIG. 22).

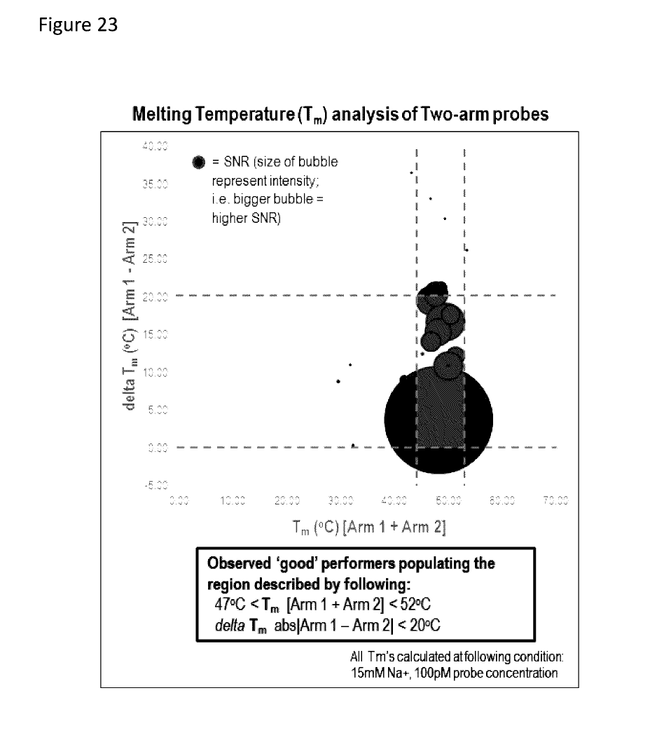

These data (especially shown in FIG. 19) indicate that the lengths of target binding regions can be adjusted to improve specificity. Without being bound by theory, it is possible to predict “well-performing” probes based on an association including the difference in melting temperature between the first target binding region and the second target binding region and including the sum of melting temperature for the first target binding region and the second target binding region. For selected data of this Example, FIG. 23 shows that well-performing probes have Tm differences of less than 20° C. (e.g., between about 0° C. and about 10° C.) and have sums of the Tms of between about 40° C. and about 60° C. (e.g., between about 47° C. and about 52° C.), when Tms are calculated under 15 mM Na⁺ and 100 pM probe concentration conditions. “SNR” stands for Signal to Noise Ratio.

Experiments detecting the EGFR gene's T790M SNP were also performed.

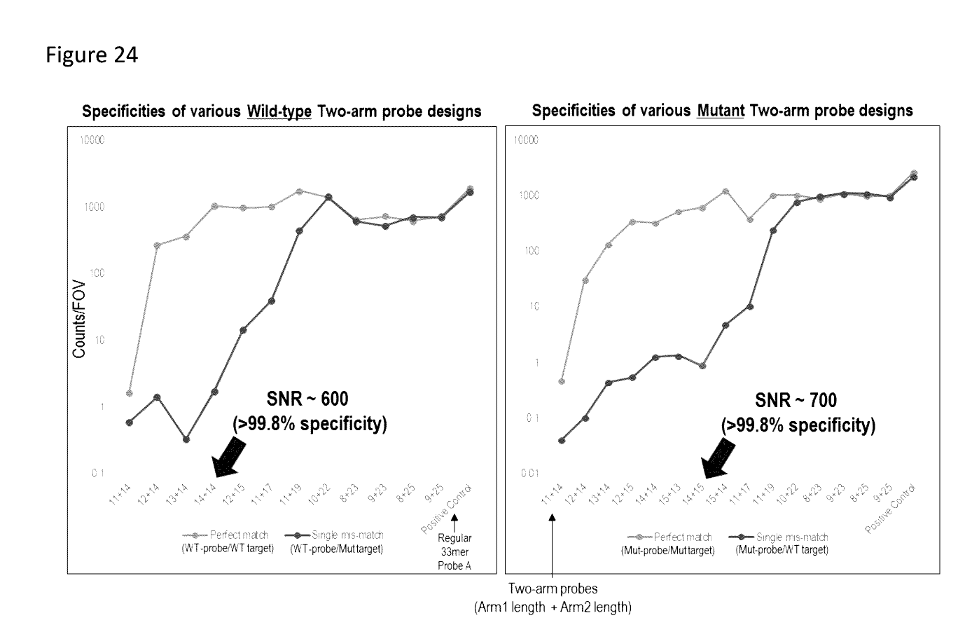

A variety of probes having target binding domains of different lengths (i.e., 8+23, 8+25, 9+23, 9+25, 10+22, 11+14, 11+17, 11+19, 12+14, 12+15, 13+14, 14+14, 14+15, 15+13, and 15+14) were tested. Excellent results (>99.8% specificity) for wild-type allele detection for one probe and >99.8% specificity for mutant allele detection for another probe (see, the purple bands of FIG. 24).

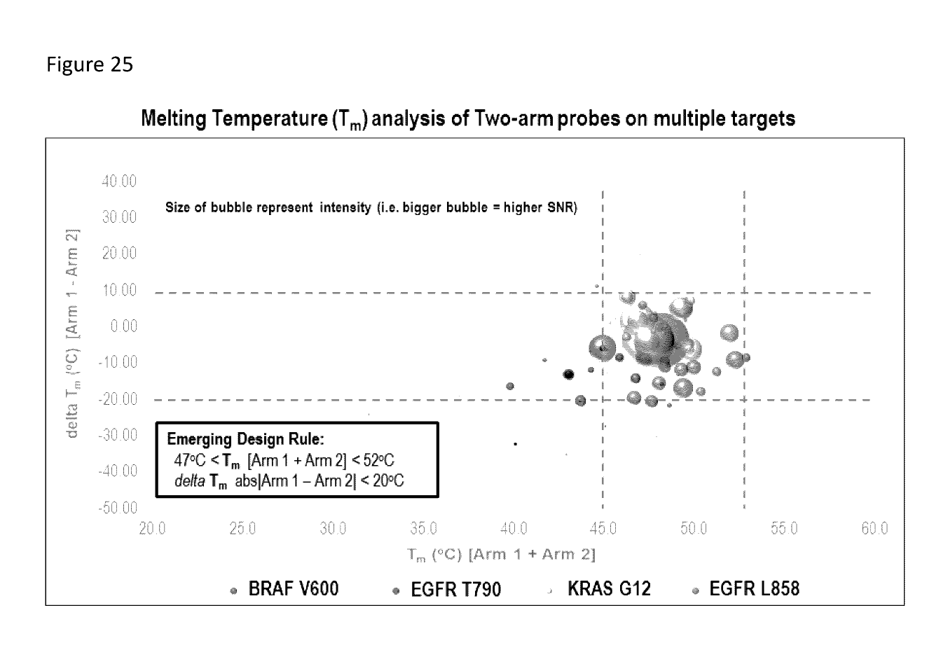

Probe design trends for well-performing two-armed probes were identified based upon results obtained from four experiments. FIG. 25 illustrates an emerging rule for probe design based upon the RAF V600E SNP experiments, the EGFR T790M SNP experiments, and two other SNP detection experiments directed to KRAS G12 and EGFR L858 (data not shown).

As shown in FIG. 25, the well-performing probes have a Tm difference of less than 20° C. and a sum of the Tms of between about 47° C. and about 52° C.

The preceding example is put forth so as to provide those of ordinary skill in the art with a complete disclosure and description of how to practice the present invention, and are not intended to limit the scope of what the inventors regard as the invention. Efforts have been made to ensure accuracy with respect to numbers used (e.g., amounts and concentrations) but some experimental errors and deviations should be accounted for. Unless indicated otherwise, temperature is in degrees Centigrade and pressure is at or near atmospheric.

### Example 2

### SNV Detection Experiments in a Hotspot Region Using Polymer Strand Pairs/Partially Double-Stranded Probes with Existing DV2 Reporter Probes from NanoString Technologies®

Experiments detecting single nucleotide variants (SNV) in the KRAS exon 2 Hotspot were performed.

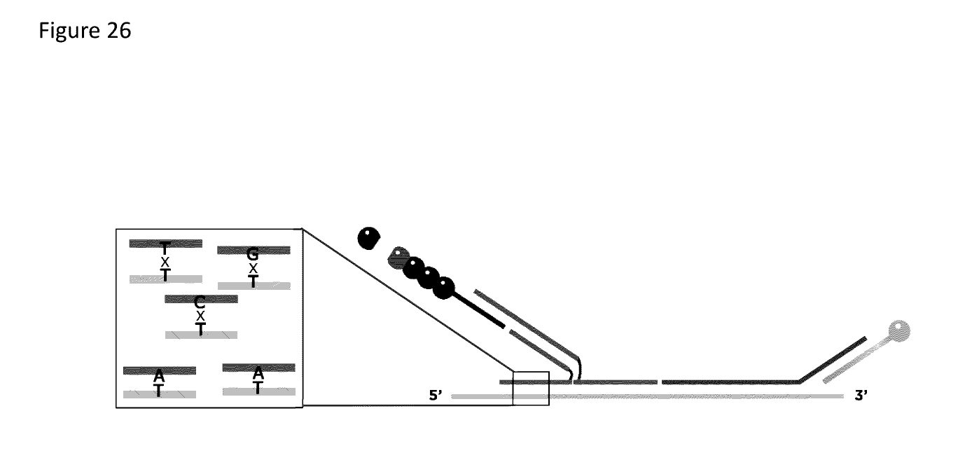

FIG. 26 shows a cartoon of the partially double-stranded probe similar to that used in this Example. In this experiment, the NanoString Technologies® DV2 system was used. Note that any probe, probe pair, or composition shown in FIGS. 1 to 15 may substitute for the probe shown in FIG. 26 for this example or any example disclosed here. In FIG. 26, Single nucleotides on a target sequence (grey) can be detected with existing NanoString Reporter oligos (green) using a two-arm adapter probe called “Probe A” (blue). The adapter probe consists of two oligos hybridized together via a stem sequence. In these experiments, both oligos hybridize to adjacent regions on the target sequence. Additionally, one of the oligos hybridizes to a NanoString Reporter oligo. There may be a PEG linker between the probe stem and hybridizing regions of each oligo. Here, single nucleotide specificity was achieved by the two-arm probe since a single nucleotide mismatch will disrupt probe hybridization. The target sequence was captured to the surface by existing NanoString technologies including an adapter “Probe B” (red) and Capture Probe (orange).

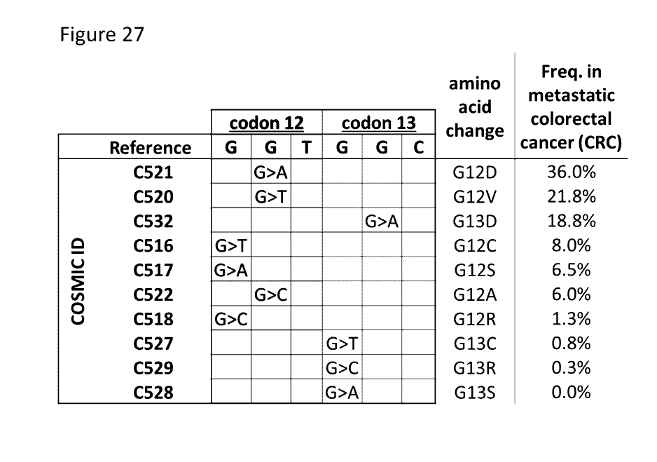

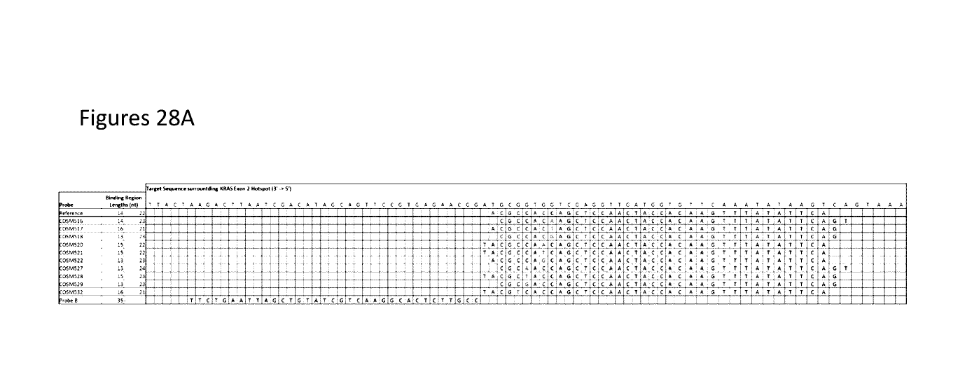

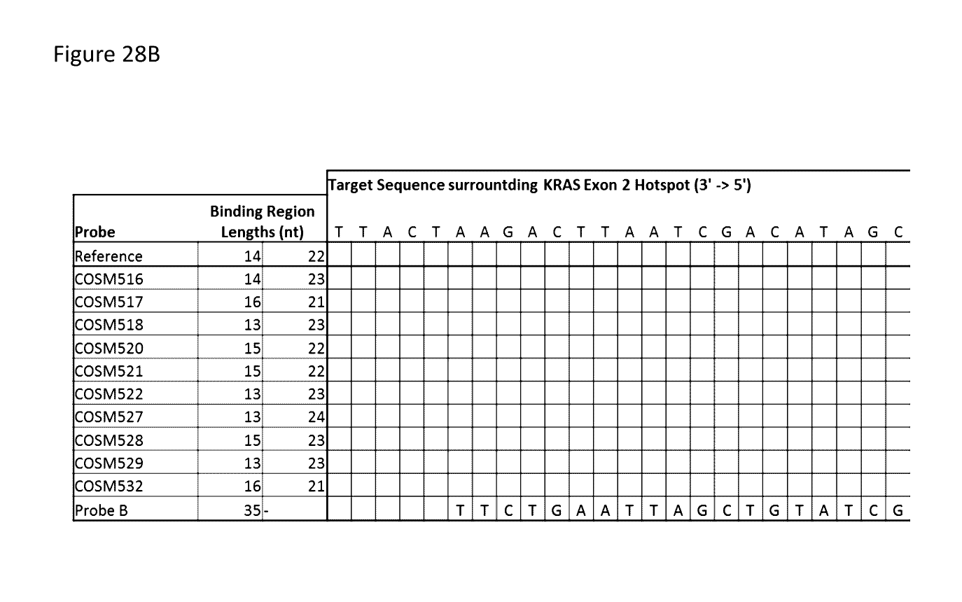

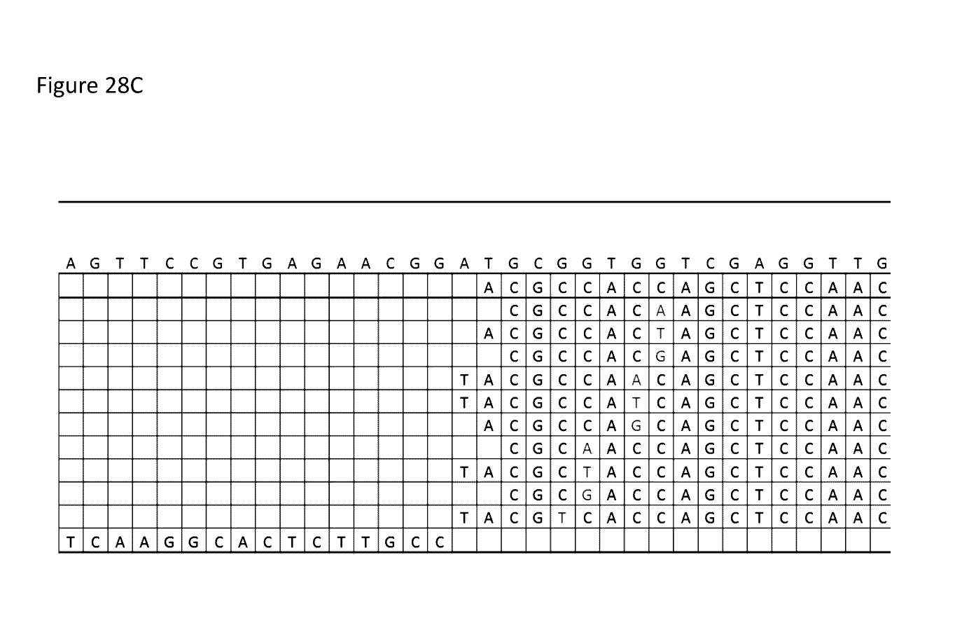

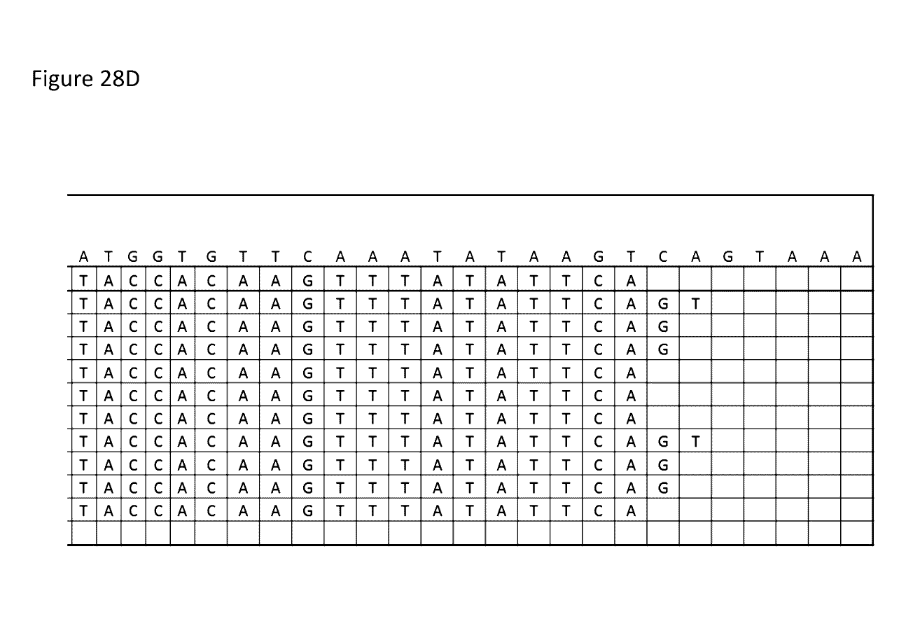

Probes specific for the Reference Sequence and SNV loci shown in FIG. 27 (KRAS exon 2 including codons 12 and 13 which contain several known single nucleotide variants) were tested. Each probe included two target binding regions that were between 13 and 24 nucleotides in length, with the SNV mutation in the shorter of the two regions. The sequences and positions of each probe are shown in FIGS. 28A to D (FIG. 28A shows the entire sequence and FIGS. 28 B to D each show one third of the sequence of FIG. 28A). In total, a probe pool contained 11 probes specific for KRAS exon 2 and 23 probes non-specific for KRAS exon 2 (background probes).

Synthetic targets were used to test the specificity of each probe in the probe pool. The probe pool was tested separately against each of the 11 KRAS target variants including the Reference Sequence and 10 SNV mutants. Background target was included for each reaction.

Hybridization reactions were performed using existing NanoString Technologies® Protocols and Reagents and were analyzed using NanoString Technologies® nCounter® Analysis System. The hybridization reaction included 25 pM of NanoString DV2 Reporters, 100 pM each Probe B, 20 pM each Probe A, 50 ng salmon sperm DNA, and 3.6 million copies of synthetic target in 5×SSPE salt. Reactions were hybridized for at least 16 hours at 65° C. before being transferred to the NanoString Technologies® nCounter® Analysis System.

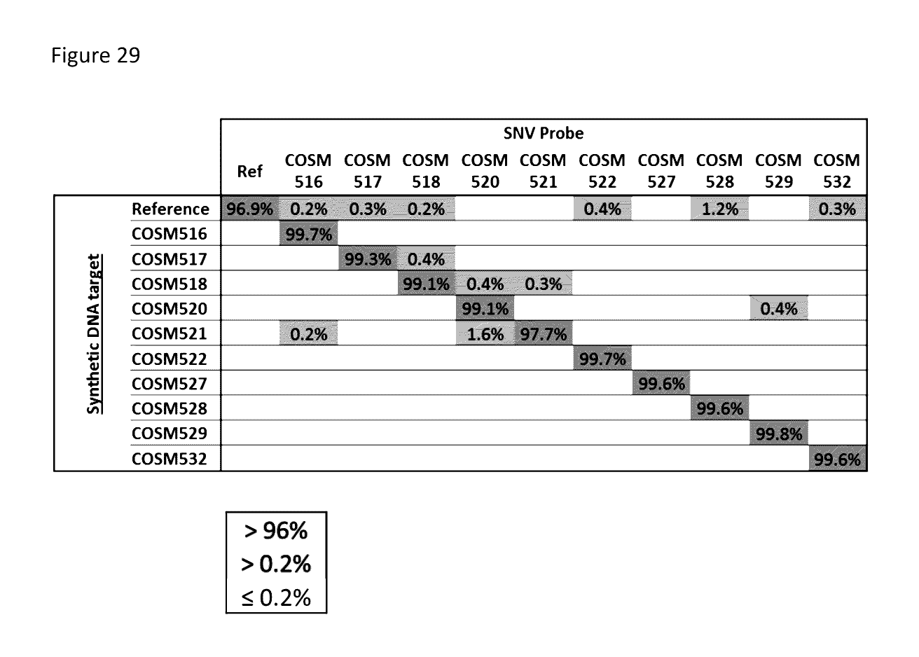

Digital counts for each probe in the probe pool revealed >96% specificity of probes for each SNV loci. Specificity was determined by the percentage of counts for a given probe to its intended target compared to counts for all KRAS targets. See, FIG. 29.

These experiments were conducted with templates containing the standard thymine (T) base as well as with template having each thymine nucleotide substituted with a uracil (U). Both template varieties produced the same results.

### Example 3

### Deletion Detection Experiments Using Polymer Strand Pairs/Partially Double-Stranded Probes with Existing DV2 Reporter Probes from NanoString Technologies®

Experiments detecting deletions in EGFR exon 19 were performed.

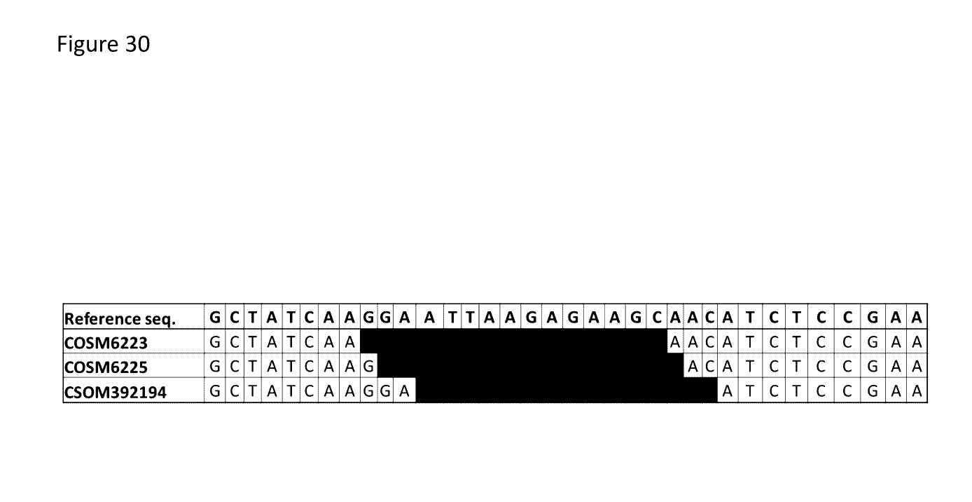

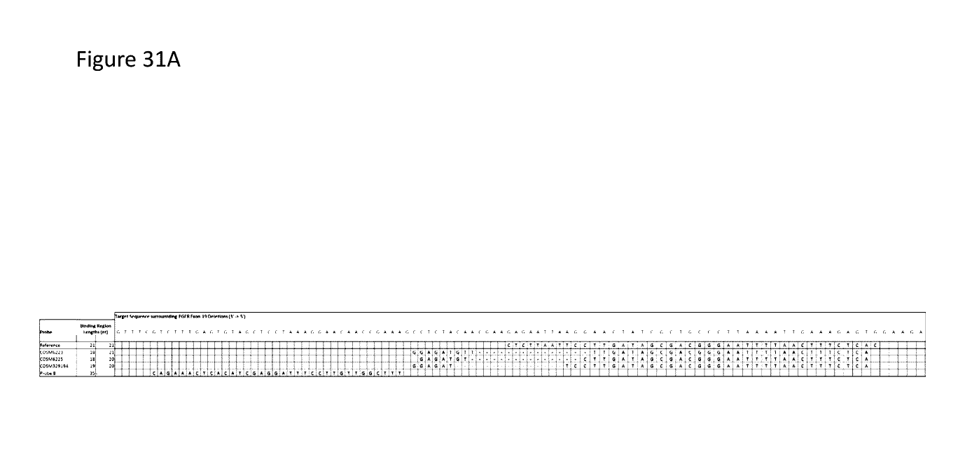

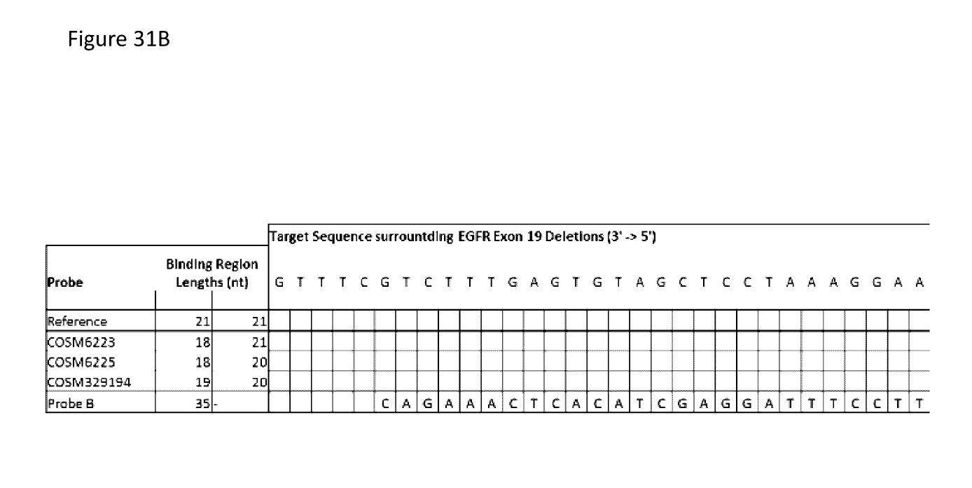

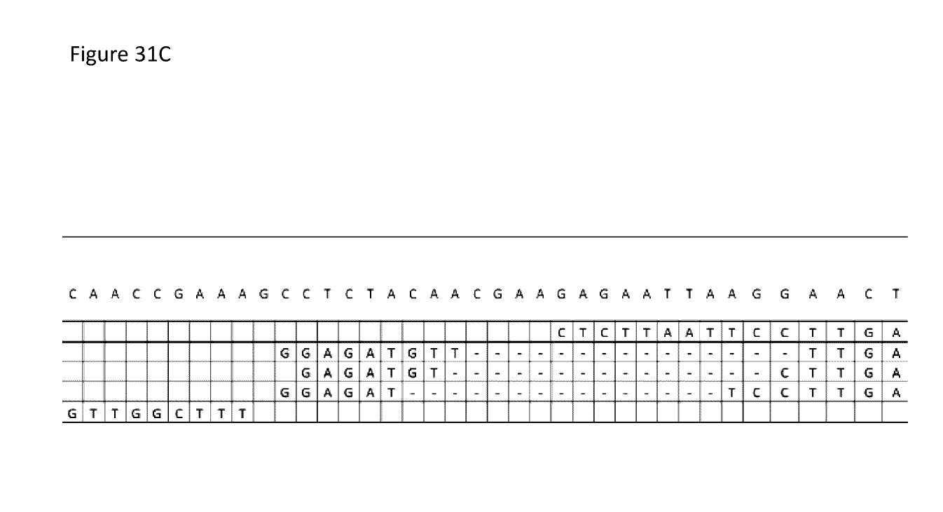

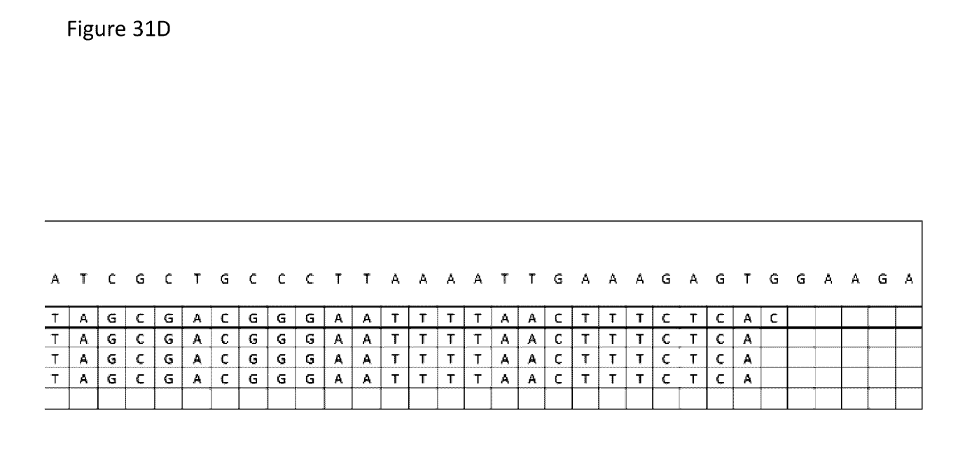

In this experiment, the NanoString Technologies® DV2 system was used with the two-arm probe architecture shown in FIG. 26. Note that any probe, probe pair, or composition shown in FIGS. 1 to 15 may substitute for the probe shown in FIG. 26 for this example or any example disclosed here. Probes specific for the Reference Sequence and three targets containing deletions shown in FIG. 30 were tested. Each probe included two target binding regions that were between 18 and 21 nucleotides in length. The sequences and positions of each probe are shown in FIGS. 31A to D (FIG. 31A shows the entire sequence and FIGS. 31B to D each show one third of the sequence of FIG. 31A). In total, a probe pool contained 4 probes specific for EGFR exon 19 and 11 probes non-specific for EGFR exon 19 (background probes).

Synthetic targets were used to test the specificity of each probe in the probe pool. The probe pool was tested separately against each of the 4 EGFR target variants, including the Reference Sequence and 3 deletion mutant sequences. Background target was included for each reaction.

Hybridization reactions were performed using existing NanoString Technologies® Protocols and Reagents and were analyzed using NanoString Technologies® nCounter® Analysis System. The hybridization reaction included 25 pM of NanoString DV2 Reporters, 100 pM each Probe B, 20 pM each Probe A, 50 ng salmon sperm DNA, and 3.6 million copies of synthetic target in 5×SSPE salt. Reactions were hybridized for at least 16 hours at 65° C. before being transferred to the nCounter® Analysis System.

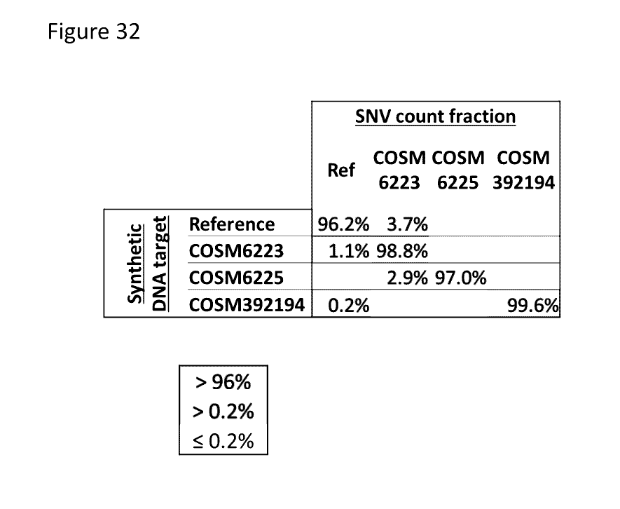

Digital counts for each probe in the probe pool revealed >96% specificity of probes for each SNV loci. Specificity was determined by the percentage of counts for a given probe to its intended target compared to counts for all EGFR targets. See, FIG. 32.

### Example 4

### Multiplex SNV Detection Experiments Using Polymer Strand Pairs/Partially Double-Stranded Probes with Existing DV2 Reporter Probes from NanoString Technologies®

Experiments detecting multiple SNV mutations in a single reaction were performed.

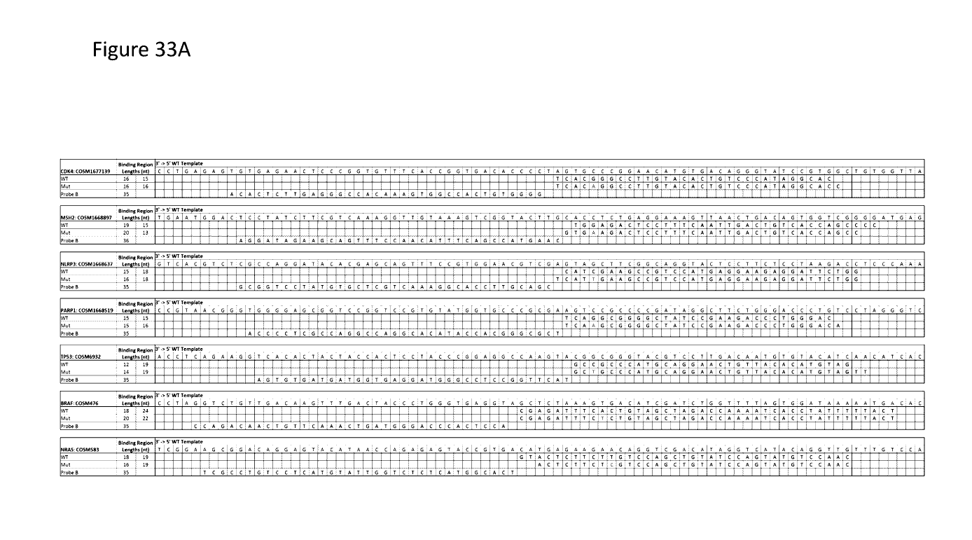

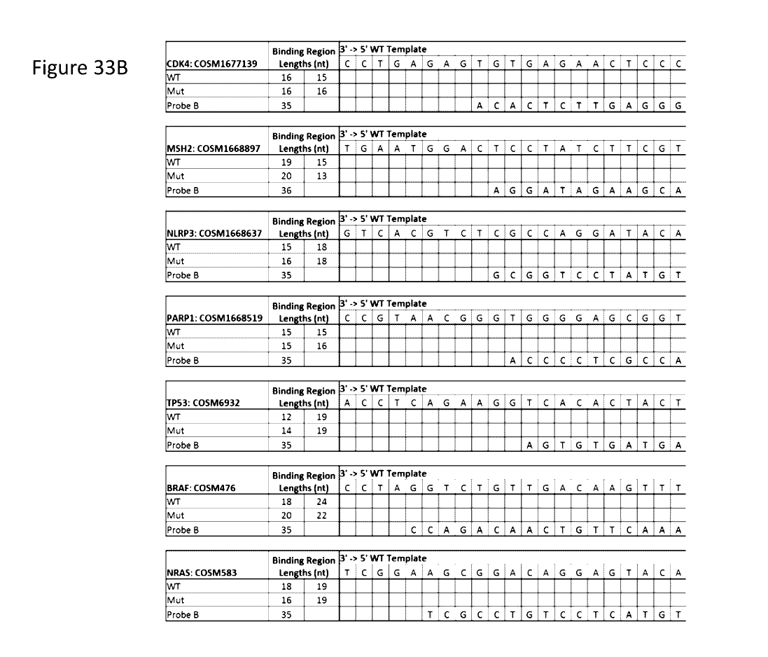

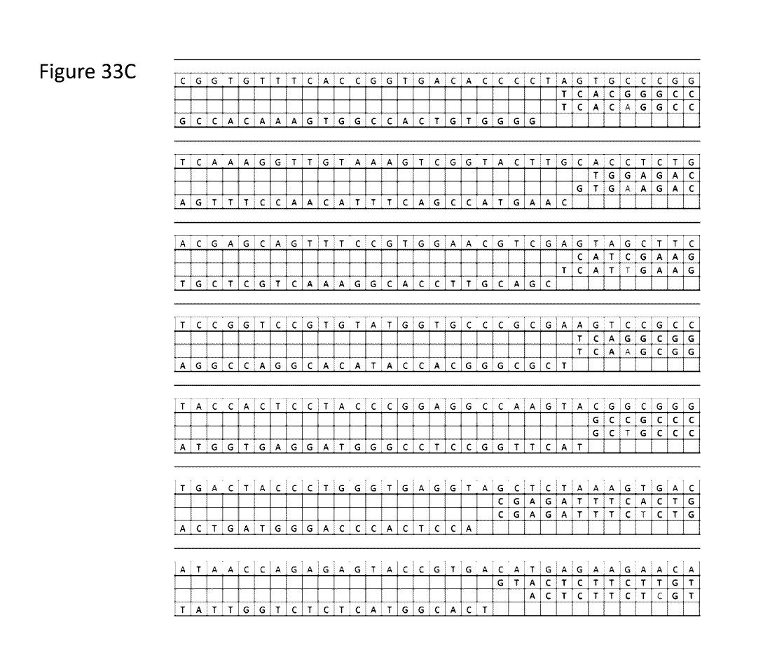

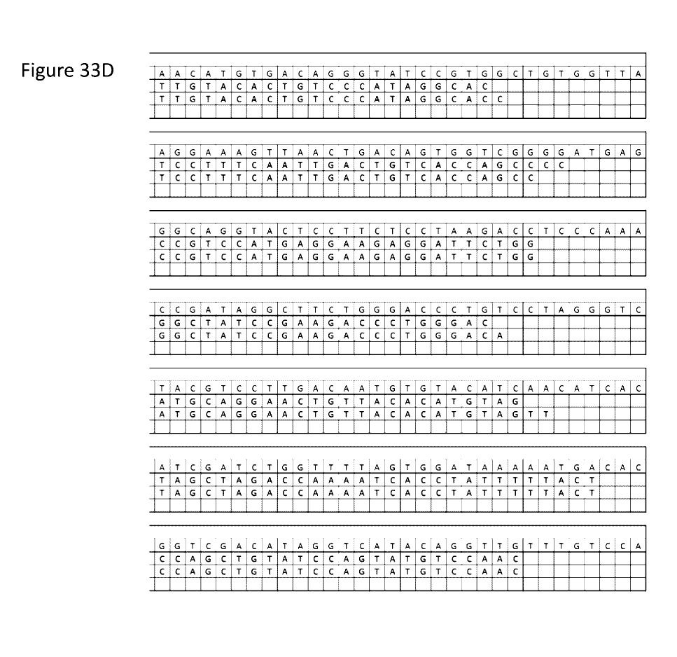

In this experiment, the NanoString Technologies® DV2 system was used with the two-arm probe architecture shown in FIG. 26. Note that any probe, probe pair, or composition shown in FIGS. 1 to 15 may substitute for the probe shown in FIG. 26 for this example or any example disclosed here. Probes specific for Reference and SNV Sequences at 7 different loci were tested. Each probe included two target binding regions that were between 18 and 21 nucleotides in length. The sequences and positions of each probe are shown in FIGS. 33A to D (FIG. 33A shows the entire sequence and FIGS. 33B to D each show one third of the sequence of FIG. 33A). In total, a probe pool contained 14 two-arm probes and 1 single arm probe for RNAseP that acted as an endogenous control.

DNA was extracted from three cell lines using the DNeasy Blood & Tissue Kit from Qiagen®. 20 ng of DNA was amplified in a 20 μl PCR reaction using TaqMan™ Hotstart 2× Mastermix with 200 nM forward and reverse Primer oligos and the following thermocycler program: (1) 95° C. for 30 sec, (2) 95° C. for 15 sec, (3) 56° C. for 30 sec, (4) 68° C. for 30 sec, (5) repeat steps (2) to (4) for 18 cycles, and (6) 68° C. for 300 sec. Prior to use, amplified DNA was denatured by heating to 95° C. for 10 min and then rapidly cooling on ice.

The probe pool was tested separately against amplified DNA from each of the 3 cell lines to determine the genotype of each of the 7 loci of interest.

Hybridization reactions were performed using existing NanoString Technologies® Protocols and Reagents. The hybridization reaction included 25 pM of NanoString DV2 Reporters, 100 pM each Probe B, 20 pM each Probe A, and 5 uL amplified DNA in 5×SSPE salt. Reactions were hybridized for at least 16 hours at 65° C. before being transferred to the nCounter® Analysis System.

In these experiments, the hybridization reaction described here was combined with a separate hybridization reaction containing a NanoString Technologies nCounter PanCancer Profiles gene expression panel with Protein Plus. The combined hybridization reactions were run on the NanoString Technologies® nCounter® Analysis System.

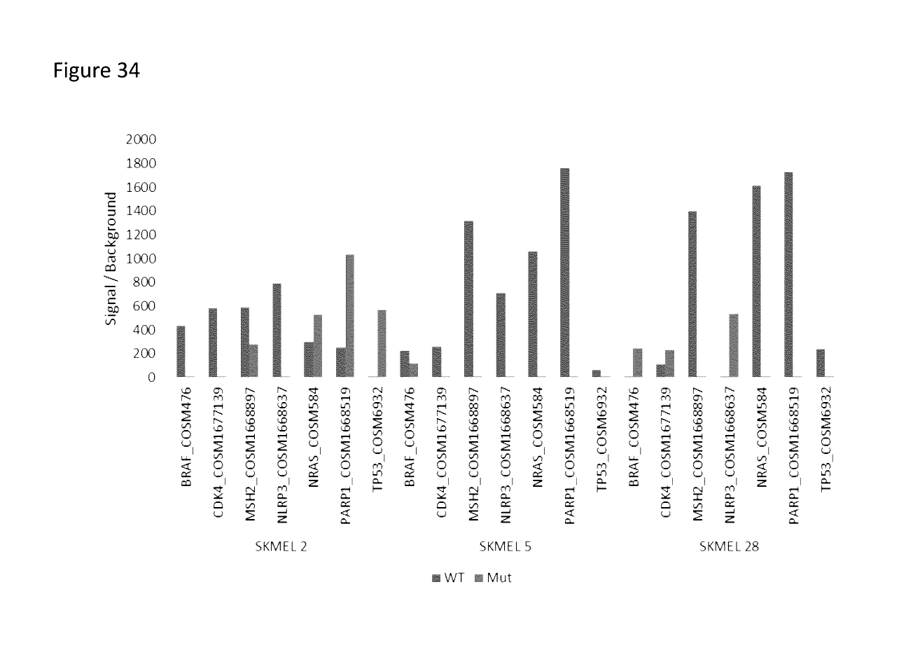

Digital counts for the reference (wild type, WT) and SNV (mutant, Mut) probes were compared and the genotype at each locus was determined by which probe showed a signal over background. See, FIG. 34.

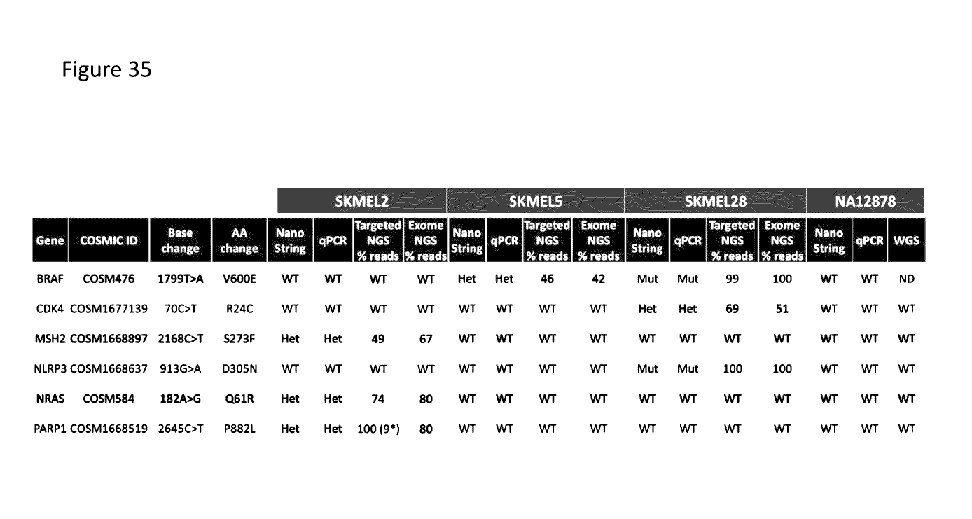

The genotype determined for each cell line and each locus using this SNV assay matched those determined by other methods including a TaqMan™ genotyping assay and published data. See, FIG. 35.

### Example 5

### 3D Biology: Simultaneously Detecting DNA SNV, RNA Gene Expression, and Protein Profiles on the Same Instrument

Experiments combining the SNV detection assay described herein with other NanoString Technologies® assays were performed.

Cells from each of three cell lines were divided for DNA, RNA, and protein sample preparation. After initial processing, RNA and protein were combined into a single hybridization reaction. DNA was hybridized in a separate reaction.

DNA Hybridization Reaction:

DNA was extracted using the DNeasy Blood & Tissue Kit from Qiagen®. Extracted DNA was either amplified as described in Example 3 or sheared into 300 bp segments using the Covaris instrument. Prior to use, DNA was denatured by heating to 95° C. for 10 min then rapidly cooling on ice. The NanoString Technologies® DV2 system was used with the two-arm probe architecture shown in FIG. 26. Note that any probe, probe pair, or composition shown in FIGS. 1 to 15 may substitute for the probe shown in FIG. 26 for this example or any example disclosed here. The genotype of a SNV of interest was determined using probes specific for wild-type and mutant sequences as described in Example 3. Hybridization reactions were performed using existing NanoString Technologies® Protocols and Reagents. The hybridization reaction included 25 pM of NanoString DV2 Reporters, 100 pM each Probe B, 20 pM each Probe A, and 5 μL amplified DNA (or 500 ng unamplified, Covaris Sheared DNA) in 5×SSPE salt. Reactions were hybridized for at least 16 hours at 65° C.

RNA: Protein Hybridization Reaction:

Protocols for combing RNA and Protein in the same hybridization are outlined in protocols for existing NanoString Technologies Products. RNA was extracted using the RNeasy Kit from Qiagen® or taken directly from lysed cells. For Protein processing, cell lysate was prepared with lysate buffer (2% SDS, 100 mM Tris pH 6.8, 50 mM DTT), added to a protein binding plate, and incubated with DNA tagged antibodies specific for proteins of interest, as in the NanoString Technologies Protein Assay. After unbound antibodies were washed away, those remaining were suspended in RLT buffer. This RLT buffer was used at the Protein Target in a standard NanoString Technologies® nCounter® hybridization reaction. Hybridization reactions were performed using existing NanoString Technologies® Protocols and Reagents including nCounter Reporters specific for both RNA and Protein Targets. Reactions were hybridized for at least 16 hours at 65° C.

The DNA hybridization reaction and RNA:Protein Hybridization reaction were combined just prior to running them on the NanoString Technologies® nCounter® Analysis System.

Digital counts for DNA SNV Probes were simultaneously measured with counts for RNA gene expression probes and Protein probes.

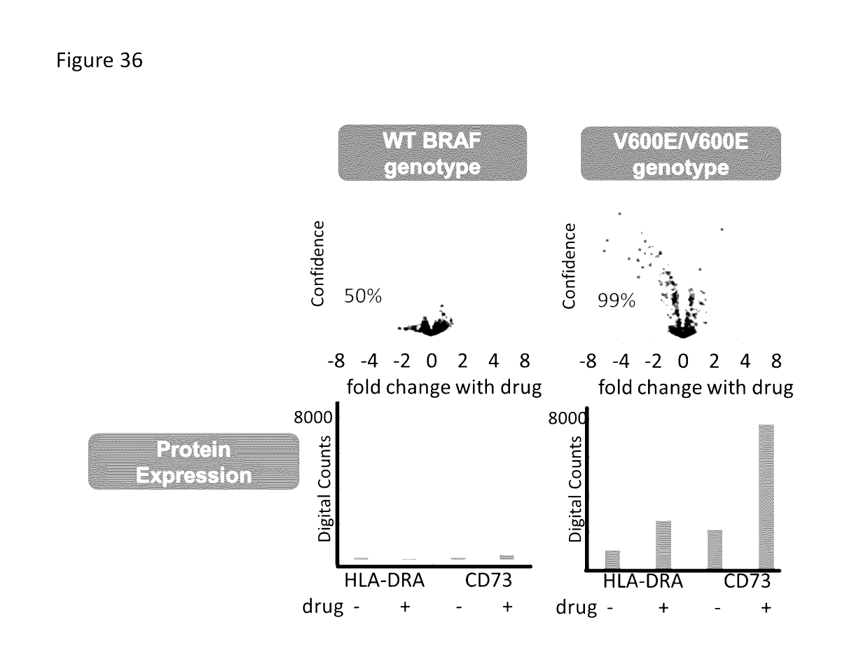

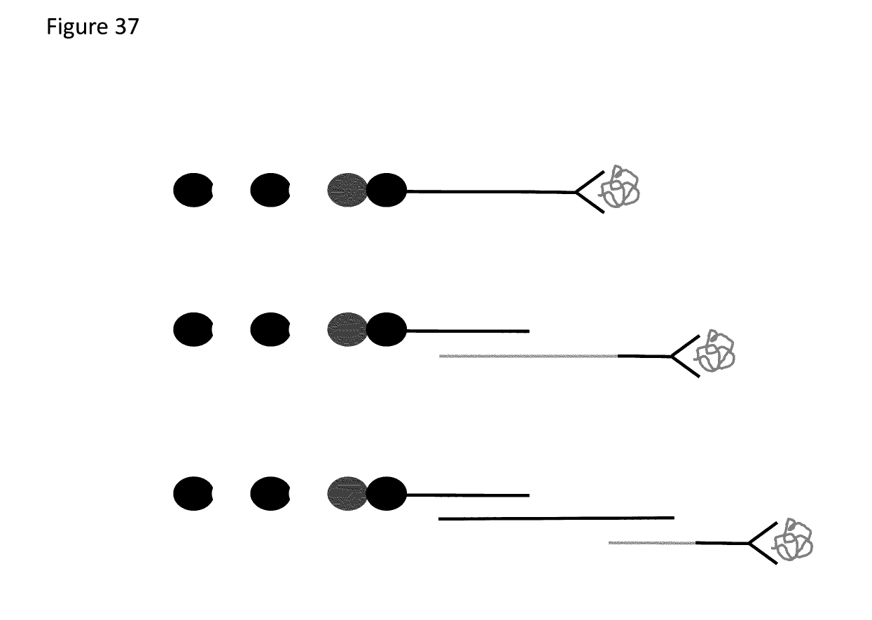

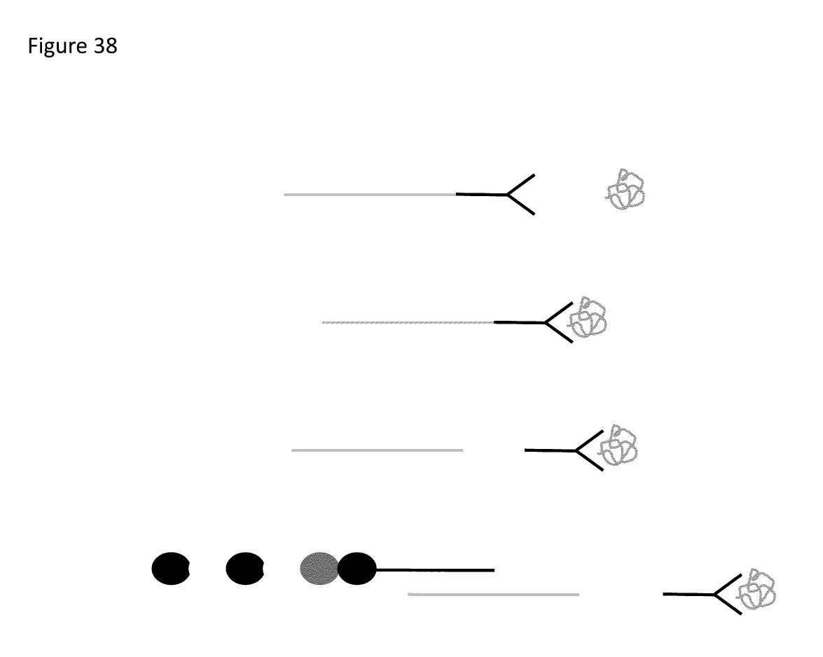

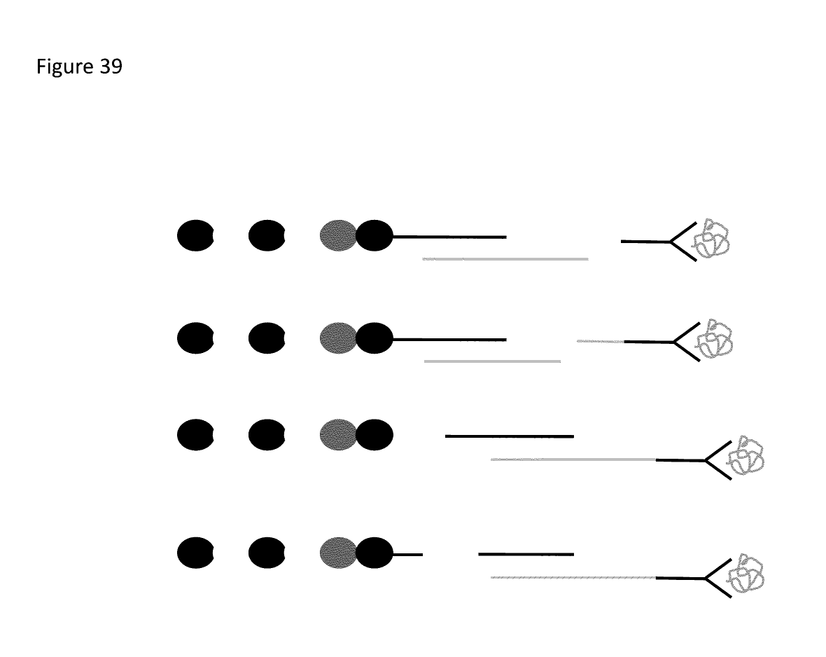

Using this technique, multiple dimensions of information can be simultaneously determined from the same sample. See, FIG. 36. Exemplary probes useful for detecting proteins and methods of use are shown in FIGS. 37 to 39.

### Example 6

### SNV Detection Experiments in a Hotspot Region Using Polymer Strand Pairs/Partially Double-Stranded Probes with Existing DV2 Reporter Probes from NanoString Technologies®

Experiments detecting single nucleotide variants (SNV) in the KRAS exon 2 Hotspot were performed.

FIG. 26 shows a cartoon of the partially double-stranded probe similar to that used in this Example. In this experiment, the NanoString Technologies® DV2 system was used. Note that any probe, probe pair, or composition shown in FIGS. 1 to 15 may substitute for the probe shown in FIG. 26 for this example or any example disclosed here. Probe specificity for the Reference Sequence and SNV Variant Sequences was tested. Each probe included two target binding regions that were between thirteen and twenty-three nucleotides in length. For the KRAS exon 2 locus, a probe pool containing one reference probe and twelve probes specific to twelve different variant sequences were used to assay the region. These probes were used in a probe pool with 64 other reference probes and 136 other variant probes, specific to other regions of the genome.

Synthetic targets containing deoxyUracil (dU) in place of deoxyThymine were used to test the specificity of each probe in the probe pool. dU-containing templates mimicked the PCR product produced in the amplification step of the SNV assay. The probe pool was tested against 3.6 million copies of each of the twelve KRAS target variants and 72 million copies of the Reference Sequence in separate reaction wells. This approximated 5% sensitivity in the context of large numbers of reference sequences. Most reactions had one variant template present, but one reaction had no variant templates present to simulate a reference sample. One reaction had two variant templates present.

Hybridization reactions were performed using existing NanoString Technologies® protocols and reagents. The hybridization reaction included 20 pM of NanoString Technologies® DV2 Reporters, 100 pM of each ProbeT, 20 pM each of 149 variant SNV ProbeS, 100 pM of each of 64 reference ProbeS, and 5 μL amplified DNA in 5×SSPE salt. Additionally, 200 pM of ProbeM (Attenuator oligo) was used to dampen superfluous reference signal and to ensure 5% sensitivity on each SNV assay. Attenuator oligos were standard 35-mer oilgos which are reverse complements to the DV2 Reporter tag; they blocked the ProbeS hybridization site. Reactions were hybridized for sixteen hours at 65° C. before being transferred to the nCounter® Analysis System.

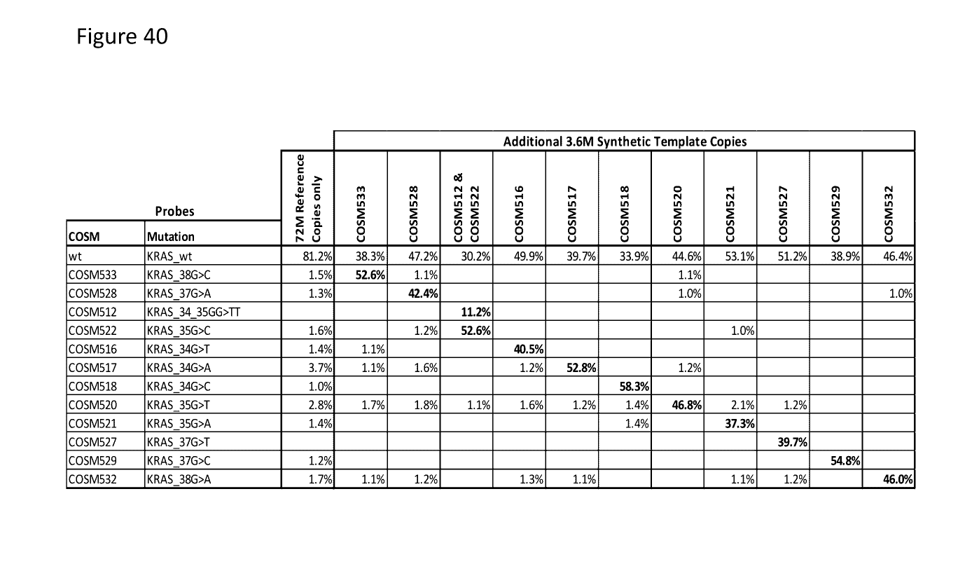

Digital counts for each probe in the EGFR exon 19 probe group revealed consistent reference signal in each reaction, and secondary counts from the variant probe specific for the exact variant template in the reaction. The distribution of total counts was calculated as an estimate of specificity for a given probe to its intended target. See, FIG. 40.

### Example 7

### Deletion Detection Experiments Using Polymer Strand Pairs/Partially Double-Stranded Probes with Existing DV2 Reporter Probes from NanoString Technologies®

Experiments detecting Insertion-Deletions in EGFR exon 19 were performed.

In this experiment, the NanoString Technologies® DV2 system was used with the two-arm probe architecture shown in FIG. 26. Note that any probe, probe pair, or composition shown in FIGS. 1 to 15 may substitute for the probe shown in FIG. 26 for this example or any example disclosed here. Each probe included two target binding regions that were between seventeen and twenty-four nucleotides in length. In total, for the EGFR exon 19 locus, a probe pool containing one reference probe and nine variant probes specific to nine variant sequences were used to assay the region. These probes were used in conjunction with 64 other reference probes and 139 other variant probes, specific to other regions of the genome.

Synthetic targets containing deoxyUracil in place of deoxyThymine were used to test the specificity of each probe in the probe pool. This mimicked the PCR products produced in the amplification step of the SNV assay. The probe pool was tested against 3.6 million copies of each of the nine EGFR exon 19 target variants and 72 million copies of the Reference Sequence in separate reaction wells. This was used to approximate 5% sensitivity in the context of large numbers of reference sequences. Most reactions had one variant template present, but one reaction was included which contained no variant templates present to simulate a reference sample.

Hybridization reactions were performed using existing NanoString Technologies® Protocols and Reagents. The hybridization reaction included 20 pM of NanoString DV2 Reporters, 100 pM of each ProbeT, 20 pM each of 149 variant SNV ProbeS, 100 pM of each of 64 reference ProbeS, and 5 μL amplified DNA in 5×SSPE salt. Additionally, 200 pM of ProbeM (Attenuator oligo) was used to dampen superfluous reference signal and to ensure 5% sensitivity on each SNV assay. Attenuator oligos were standard 35-mer oilgos which are reverse complements to the DV2 Reporter tag, they blocked the ProbeS hybridization site. Reactions were hybridized for sixteen hours at 65° C. before being transferred to the nCounter® Analysis System.

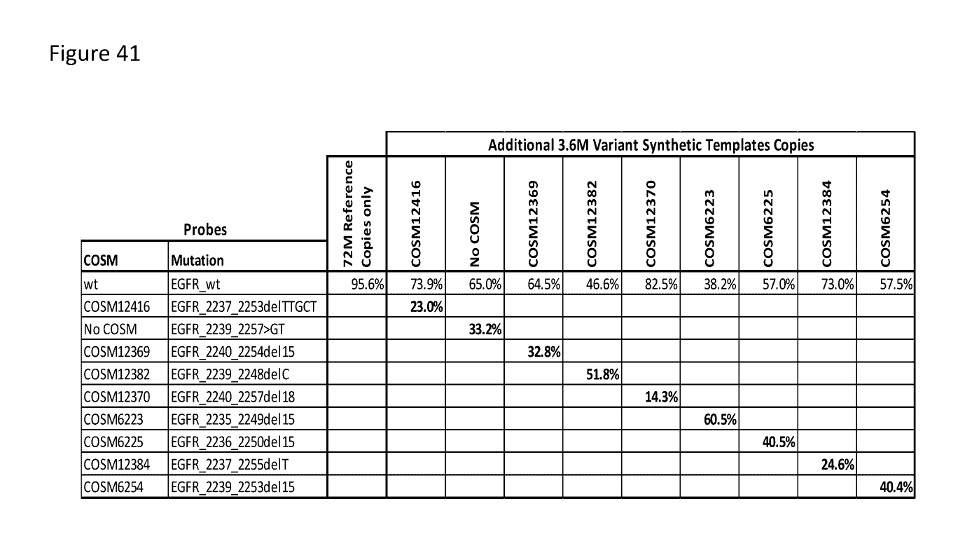

Digital counts for each probe in the EGFR exon 19 probe group revealed consistent reference signal in each reaction, and secondary counts from the variant probe specific for the exact variant template in the reaction. The distribution of total counts was calculated as an estimate of specificity for a given probe to its intended target. See, FIG. 41.

### Example 8

### Multiplex SNV Detection Experiments in a Hotspot Using Polymer Strand Pairs/Partially Double-Stranded Probes with Existing DV2 Reporter Probes from NanoString Technologies®

Experiments detecting multiple SNV mutations in a single reaction were performed.

In this experiment, the NanoString Technologies® DV2 system was used with the two-arm probe architecture shown in FIG. 26. Note that any probe, probe pair, or composition shown in FIGS. 1 to 15 may substitute for the probe shown in FIG. 26 for this example or any example disclosed here. Probes specific for Reference and SNV Sequences at forty-three different loci were tested. Each probe included two target binding regions that were between thirteen and twenty-seven nucleotides in length. In total, a probe pool contained 178 different two-arm probes and various endogenous and exogenous standard probes working as controls.

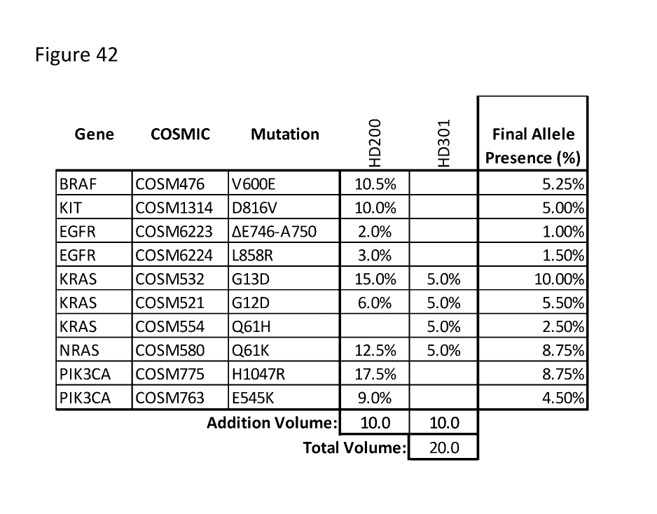

Purified genomic DNA (gDNA) samples were extracted from formalin-fixed paraffin embedded (FFPE) sections purchased from Horizon Discovery, each engineered to contain four or nine SNVs at frequencies between 2% and 17.5%. By pooling two products together (HD200+HD 301), a sample was created which contained 10 SNVs of the 114 SNVs assayed in the panel across 43 targets. Each mutant would be present at 1-10%, as shown in FIG. 42.

As a reference sample, Coriell sample NA12878 was used. This sample has been found to show only reference signal at all loci assayed.

5 ng of each DNA was amplified using forty-three custom primer pairs to amplify SNV loci of interest using the following thermocycler program: (1) 37° C. for 30 min, (2) 50° C. for 600 sec, (3) 95° C. for 180 sec, (4) 95° C. for 30 sec, (5) 56° C. for 120 sec, (7) 68° C. for 30 sec, (8) repeat steps (4) to (7) for 21 cycles, and (9) 68° C. for 300 sec. Prior to use, amplified DNA was denatured by heating to 95° C. for 10 min and then rapidly cooling on ice.

Hybridization reactions were performed using existing NanoString Technologies® protocols and reagents. The hybridization reaction included 25 pM of NanoString Technologies® DV2 Reporters, 100 pM each ProbeT, 20 pM each of 114 variant SNV ProbeS, 100 pM of each of 43 reference ProbeS, and 5 μL amplified DNA in 5×SSPE salt. Additionally, 200 pM of ProbeM (Attenuator oligo) was used to dampen excessive reference signal and allow for a number of cycles to reach at least 5% sensitivity on each SNV. Attenuator oligos are standard 35-mer oilgos which are reverse complements to the DV2 Reporter tag, they blocked the ProbeS hybridization site. Reactions were hybridized for sixteen hours at 65° C. before being transferred to the nCounter® Analysis System.

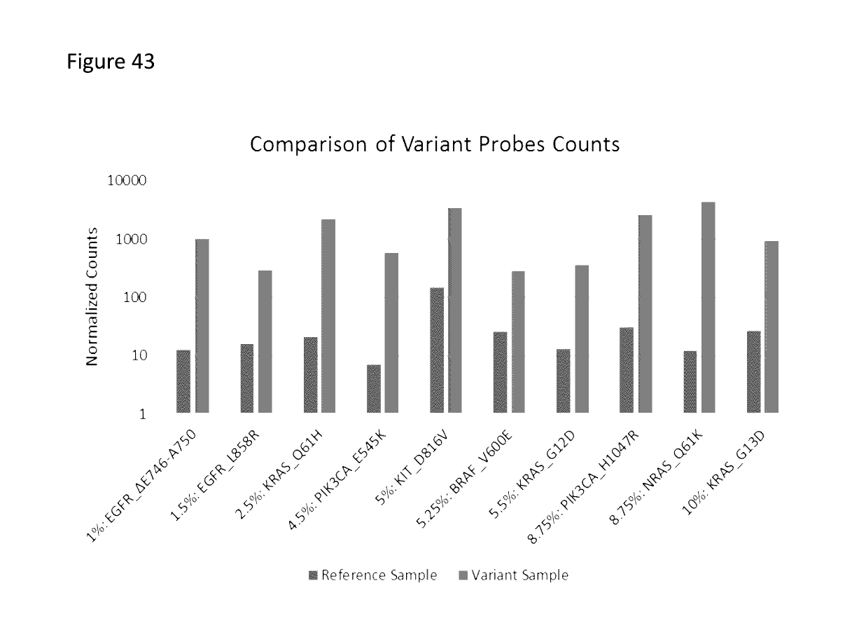

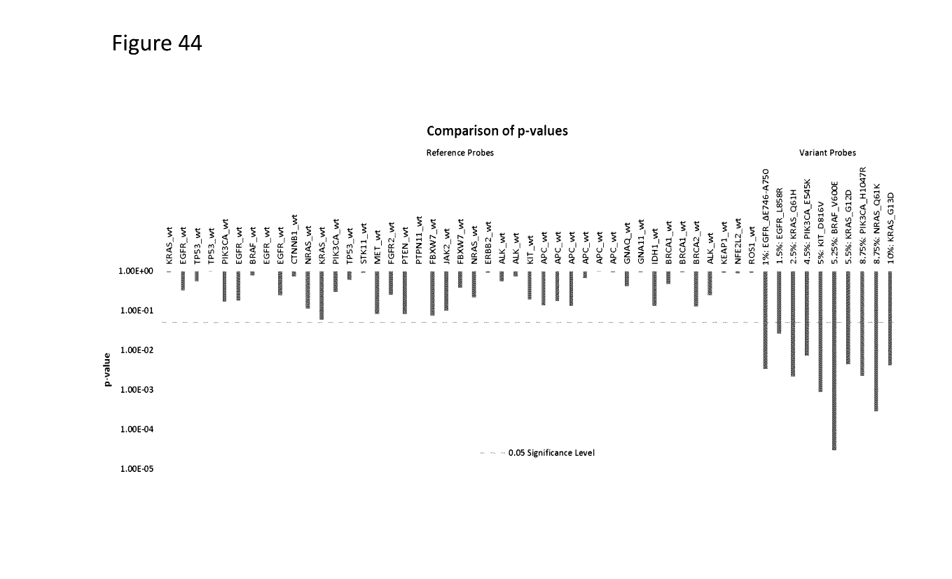

Digital counts for the reference probes and those variant probes expected to be present at detectable levels (variant, mutant) were compared in triplicate. The ten variant probes in the variant sample were shown to be significantly elevated from those same probe counts in the reference sample. See, FIG. 43. P-values were generated by tabulating Log₂ variant-to-reference ratios and calculating t-scores. All expected variant probes yielded p-values below a 0.05 significance. All reference probes yielded p-values above a 0.05 significance level. See, FIG. 44.

### Example 9

### Simultaneous SNV Detection and RNA Fusion Transcript Detection on an nCounter® System Using Polymer Strand Pairs/Partially Double-Stranded Probes with Existing DV2 Reporter Probes from NanoString Technologies and the NanoString Technologies nCounter® Lung Gene Fusion Panel

An experiment to simultaneously detect SNV mutations and the presence of RNA gene fusion transcripts associated with lung cancer was carried-out on patient-derived genomic DNA extracted from a formalin-fixed paraffin-embedded (FFPE) tissue sample and RNA extracted from the same tissue sample or RNA extracted from a commercial control sample comprised of formalin-fixed paraffin embedded (FFPE) cultured cells.

The DNA and RNA were extracted from single FFPE sections using an AllPrep® DNA/RNA FFPE Kit (Cat No. ID: 80234) from Qiagen® (Germany) following the vendor's recommended protocol.

The FFPE cultured cells were commercially obtained from Horizon Discovery Group PLC (Cambridge, England) and are described as ALK-RET-ROS1 Fusion RNA Reference Standard (Catalog ID: HD784). This commercial sample was provided as a single 10 μm thick FFPE section or “curl”. It is further described by the vendor as a highly-characterized biologically-relevant reference material composed of cell lines that were either engineered or clonally derived from a fusion background. It is additionally described as positive for an EML4-ALK fusion (variant 1; COSMIC ID: COSF463), a CCDC6-RET fusion (COSMIC ID: COSF1272), and an SLC34A2-ROS1 fusion (COSMIC ID: COSF1197).

The patient-derived FFPE sample (Specimen ID: 1194863B from Case ID: 82430) was obtained from Asterand Bioscience (Detroit, Mich.). Using genotype-specific PCR, the vendor prescreened and confirmed the sample to be positive for the KRAS p.G13D SNV (c.38G>A; COSMIC ID: COSM532) prior to use in this example. The specimen was from a lung tumor lobectomy performed on a 57 year-old non-Hispanic Caucasian male. The tumor was described as UICC Stage: T1bN0M0 and as a moderately differentiated mucinous type of adenocarcinoma of the lung (a form of non-small cell lung carcinoma (NSCLC)). The specimen was purchased as an FFPE block and individual ˜10 μm sections were cut from the block for DNA and RNA extraction from one or more sections.

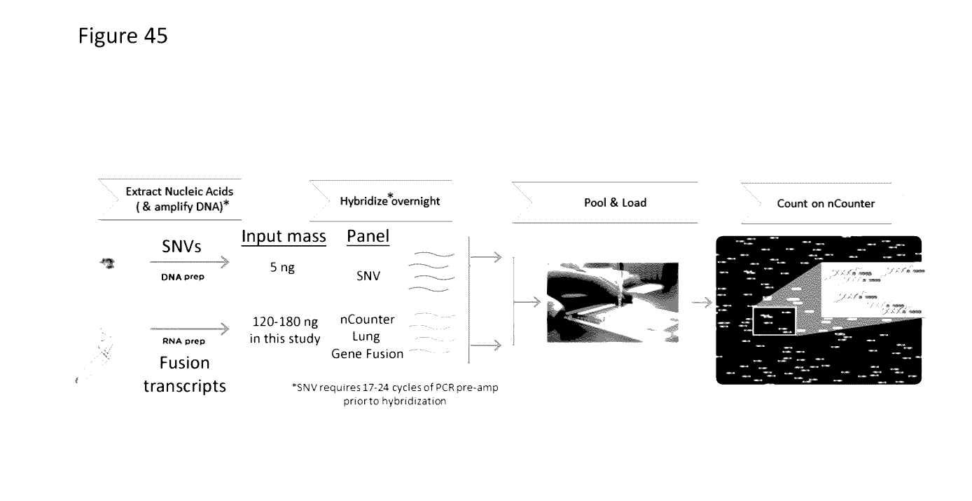

The overall experimental workflow is shown in FIG. 45. In the experiment, extracted genomic DNA (gDNA) was processed through the SNV assay workflow from extraction through pre-amplification and a sixteen-hour hybridization with the SNV-specific probe-pool. In parallel, extracted total RNA was processed with the nCounter® Vantage™ Lung Fusion Panel probes (NanoString Technologies®, Seattle, Wash.; Catalog No. XT-CSO-LKFU1-12) through a sixteen-hour hybridization per the published user protocol. After the parallel sixteen-hour hybridizations, the two hybridization reactions were pooled, mixed, and loaded into a single nCounter® cartridge lane for automated purification, immobilization, and imaging. Positive probe counts within each single lane were enumerated by automated microscopy imaging on an nCounter® system. Detection of SNVs and fusion transcripts was based on the enumeration data and was positive when counts significantly exceed the background count level.

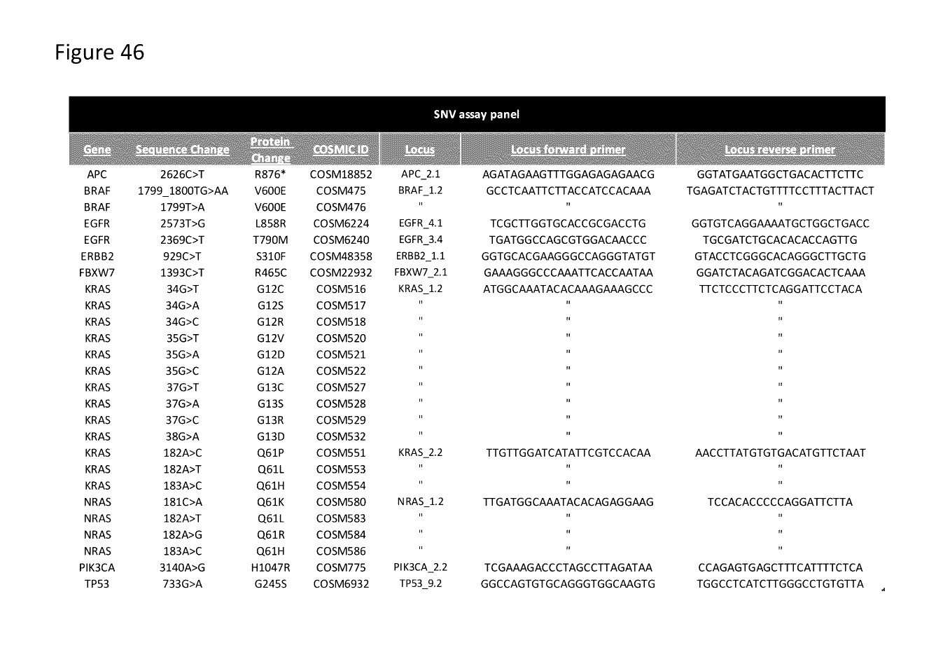

Specifically, in the embodied experiment, in a 10 μl reaction, 5 ng of FFPE-extracted DNA was amplified using the eleven primer pairs listed in FIG. 46 to amplify the SNV loci of interest using the following thermocycler program: (1) 37° C. for 30 min, (2) 50° C. for 600 sec, (3) 95° C. for 180 sec, (4) 95° C. for 30 sec, (5) 56° C. for 120 sec, (7) 68° C. for 30 sec, (8) repeat steps (4) to (7) for 20 cycles, and (9) 68° C. for 300 sec. Prior to use, amplified DNA was denatured by heating to 95° C. for 10 min and then rapidly cooled on ice.

Hybridization reactions were performed using existing NanoString Technologies® protocols and reagents. The 15 μl SNV-detection hybridization reaction included 25 pM of NanoString Technologies® DV2 Reporters, 100 pM each standard probe B (i.e., the SNV ProbeT pool), 20 pM each of twenty-six variant SNV two-arm probes (see, FIG. 311), 100 pM of each of eleven reference two-arm probes (the pool of two-arm probes is also referred to herein as a ProbeS pool), and 5 μl of amplified DNA in 5×SSPE buffer. Additionally, 200 pM of ProbeM (Attenuator oligo pool) was used to dampen excessive reference signal and permit a number of PCR cycles to be used that enables 5% sensitivity for each SNV mutant allele. Attenuator oligos are standard 35-mer oligos that are reverse complements to the DV2 Reporter tag that competitively block the two-arm probe from the DV2 Reporter hybridization sequence. Each two-arm probe included two target binding regions that were between twelve and twenty-five nucleotides in length. Additional control probes were also included in the hybridization reaction. SNV detection reactions were hybridized for sixteen hours at 65° C. before being pooled and mixed with a standard (using 120 ng RNA input) 15 μl RNA/Fusion-probe hybridization reaction that had also hybridized at 65° C. for sixteen hours. After pooling and mixing, the combined hybridization reactions were transferred to the nCounter® Analysis System for automated processing and enumeration. The following combinations of samples and assay panels were evaluated on the same nCounter® cartridge: A) SNV assay on Reference NA12878 genomic DNA (gDNA) alone, B) simultaneous SNV assay on patient-derived gDNA extracted from FFPE in combination with the nCounter® Vantage™ Lung Fusion Panel assay on the RNA extracted from the same patient-derived FFPE sample, and C) simultaneous SNV assay on patient-derived gDNA extracted from FFPE in combination with RNA extracted from Horizon Discovery ALK-RET-ROS1 Fusion RNA Reference Standard FFPE sample.

As a human genomic DNA reference sample, sample NA12878 (Coriell Institute, Camden, N.J.) was used. This sample has been found to show only reference signal at all loci assayed. 5 ng of this non-FFPE gDNA was processed as above with only the SNV probe panel (with seventeen cycles of pre-amplification PCR). After hybridization at 65° C. for sixteen hours, the hybridization reaction for this control was loaded into lane 1 of the same nCounter® cartridge onto which all other measurements were made.

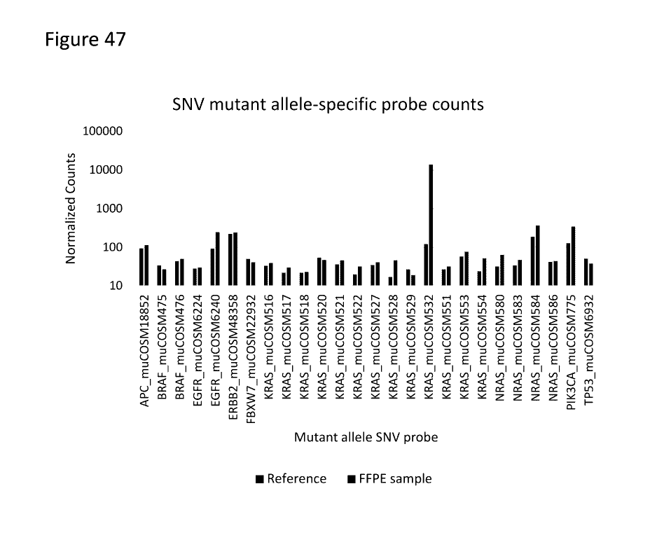

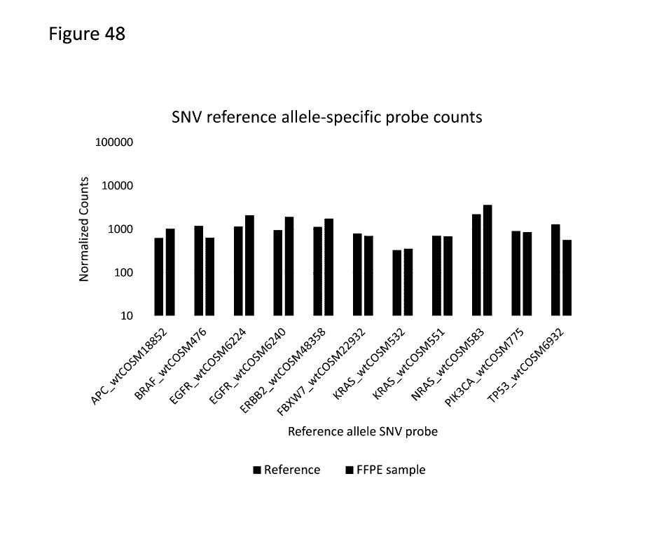

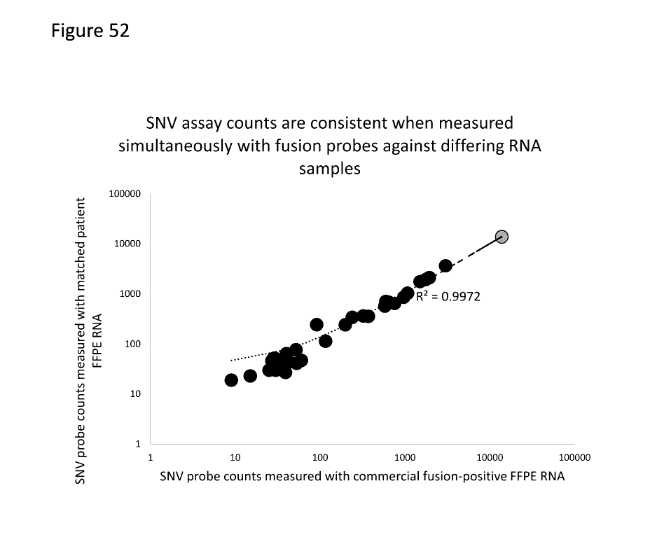

After normalization of SNV probe counts based on internal controls, digital counts for the reference allele probes and variant allele probes from the experimental samples B and C (described two paragraphs above) were compared to matched probe counts obtained from the NA12878 reference sample. Histograms of the two datasets (sample A vs. sample C in this example) revealed that the only significantly differing probe-count corresponded to the SNV probe for the KRAS COSM532 mutation in the FFPE sample, as shown in FIG. 47. In contrast, no significant probe count differences for the reference alleles were shown between these two samples, as shown in FIG. 48. This is interpreted as evidence that the FFPE-derived gDNA has reference alleles present at all interrogated loci. Qualitatively identical results were obtained when samples A and B were compared. FIG. 52 and its accompanying description below demonstrate that samples B and C yield highly correlated SNV counts.

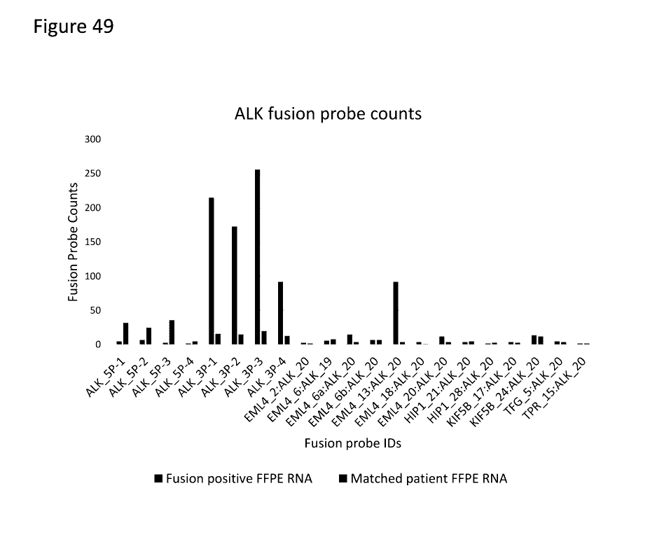

Minimally-processed fusion-probe assay counts were evaluated from the data for sample combinations B and C (as described three paragraphs above). A histogram of the counts from probes designed to detect ALK gene-derived transcripts is shown in FIG. 49. Clear evidence of 5′ vs 3′ ALK transcript imbalance were shown for the fusion positive control—counts for probes that match 3′ portions of the transcribed ALK gene, ‘ALK_3P-1’, ‘ALK_3P_2’, ‘ALK_3P_3’, and ‘ALK_3P_4’, were significantly higher than those that match 5′ portions (the ALK_5P-n series) of the same gene—and there was evidence that the specific EML4_13:ALK_20 transcript was present in the same sample; both of these results were consistent with the reported presence of an EML4-ALK fusion in this control. In contrast, there is little evidence of 5′/3′ ALK transcript imbalance or of any specific ALK fusion in the patient sample.

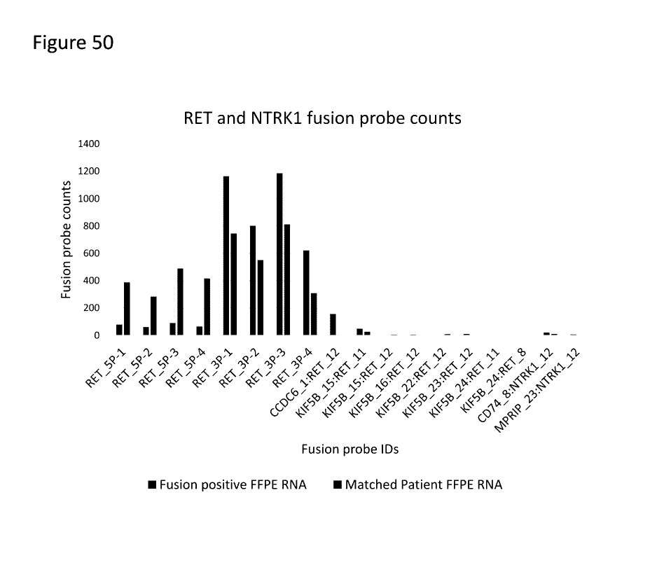

A histogram of the counts from probes designed to detect RET and NTRK1 gene-derived transcripts is shown in FIG. 50. Clear evidence of 5′ vs 3′ ALK transcript imbalance were shown for the fusion positive control and there is evidence that the specific CCDC6_1:RET_12 transcript is present in the same sample; both of these results were consistent with the reported presence of an CCDC6:RET fusion in this control. In contrast, there is little evidence of 5′/3′ RET transcript imbalance or of any specific RET or NTRK1 fusion in the patient sample.

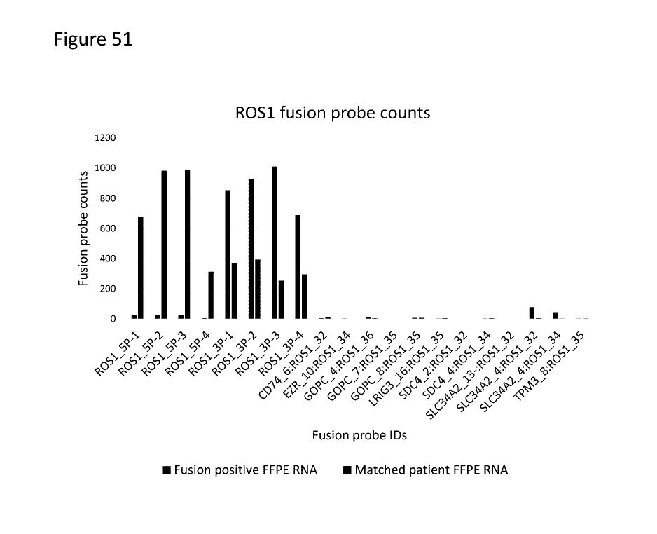

A histogram of the counts from probes designed to detect ROS1 gene-derived transcripts is shown in FIG. 51. Clear evidence of 5′ vs 3′ ROS1 transcript imbalance was shown for the fusion positive control and there is evidence that one or more specific SLC34A2_4:ROS1 transcripts were present in the same sample; both of these results were consistent with the reported presence of an SLC34A2:ROS1 fusion in this control. In contrast, there is little evidence of 5′/3′ ROS1 transcript imbalance or of any specific ROS1 fusion in the patient sample.

Failure to detect evidence of a fusion transcript in the patient-derived FFPE RNA is consistent with the observation that tumors that harbor SNV driver mutations, as this one did for the KRAS COSM532 mutation, seldom also harbor fusion-derived driver mutations, such as those associated with ALK, RET, and ROS1.

Simultaneous assay for the detection of RNA fusion-gene transcripts did not significantly affect the SNV assay probe counts. After normalization, SNV probe counts obtained from simultaneous assay of patient-matched FFPE RNA with the nCounter® Vantage′ Lung Fusion Panel closely matched SNV probe counts obtained from an analogous assay run with FFPE RNA obtained from the commercial Horizon HD784 control. This is demonstrated in FIG. 52. In the figure, the highest counts in both datasets are from the SNV probe that detected the presence of the mutant KRAS COSM532 allele (indicated by an orange dot). Reference allele SNV probes for all eleven interrogated loci occur as the next highest group of probe signals, as expected (indicated by green dots). Finally, SNP probes designed to detect absent mutant alleles in the eleven loci cluster with generally very low counts, usually below one hundred (indicated by blue dots in FIG. 52).

1. A polymer strand pair comprising:
a first polymer strand comprising at least
(1) a first target binding region,
(2) a first complementary region, and
(3) a sequence-specific region
a second polymer strand comprising at least
(1) a second target binding region, and
(2) a second complementary region;
wherein the target of the first target binding region and the target of the second target binding region are in the same nucleic acid molecule and the target of the first target binding region is non-overlapping with the target of the second target binding region; and
wherein the first complementary region is complementary to the second complementary region.

2. The polymer strand pair of claim 1, wherein the first polymer strand comprises a spacer between the first target binding region and the first complementary region or the second polymer strand comprises a spacer between the second target binding region and the second complementary region.

3. The polymer strand pair of claim 1, wherein at least one of the first target binding region, the first complementary region, the second target binding region, the second complementary region, and the sequence-specific region is a single stranded nucleic acid.

4. The polymer strand pair of claim 3, wherein the single stranded nucleic acid is DNA or RNA.

5. The polymer strand pair of claim 3, wherein the first polymer strand or the second polymer strand is a single stranded nucleic acid molecule.

6-11. (canceled)

12. The polymer strand pair of claim 2, wherein the first polymer strand comprises a spacer between the first target binding region and the first complementary region and the second polymer strand comprises a spacer between the second target binding region and the second complementary region.

13-15. (canceled)

16. The polymer strand pair of claim 1, wherein the nucleic acid molecule is a DNA molecule or is an RNA molecule.

17-18. (canceled)

19. The polymer strand pair of claim 1, wherein the nucleic acid molecule comprises at least one mutation relative to the corresponding wild-type nucleic acid molecule.

20. (canceled)

21. The polymer strand pair of claim 1, wherein the target of the first target binding region and the target of the second target binding region are separated by one or more nucleotides.

22.-26. (canceled)

27. The polymer strand pair of claim 1, wherein the target of the first target binding region and the target of the second target binding region are contiguous in the nucleic acid molecule.

28. The polymer strand pair of claim 1, wherein the target of the first target binding region or the target of the second target binding region comprises at least one mutation relative to the corresponding wild-type nucleic acid molecule.

29-31. (canceled)

32. The polymer strand pair of claim 1, wherein the first target binding region and the second target binding region are each about 5 to about 35 nucleotides in length.

33. (canceled)

34. The polymer strand pair of claim 32, wherein the length of the first target binding region and the length of the second target binding region sum to no more than about 55 nucleotides.

35. The polymer strand pair of claim 1, wherein the measured or predicted melting temperature of the first target binding region is between about 5° C. and about 35° C. and the measured or predicted melting temperature of the second target is between about 5° C. and about 35° C.

36. The polymer strand pair of claim 1, wherein the measured or predicted melting temperature of the first target binding region and the measured or predicted melting temperature the second target binding region differ by about 30° C. or less.

37-38. (canceled)

39. The polymer strand pair of claim 1, wherein the first complementary region and the second complementary region are each about 12 to about 60 nucleotides in length.

40. The polymer strand pair of claim 1, wherein the sequence-specific region comprises at least two label attachment positions covalently linked in a linear combination, wherein each label attachment position is capable of binding at least one complementary single-stranded oligonucleotide.

41-46. (canceled)

47. The polymer strand pair of claim 1, wherein the sequence-specific region is attached to at least one affinity moiety.

48. The polymer strand pair of claim 1, wherein the sequence-specific region is capable of binding to a portion of a reporter probe, the reporter probe comprising at least
a binding portion complementary to the sequence-specific region and
a single-stranded nucleic acid backbone, the backbone comprising at least two label attachment positions covalently linked in a linear combination, wherein each label attachment position is capable of binding at least one complementary single-stranded oligonucleotide.

49. The polymer strand pair of claim 48, wherein the at least one complementary single-stranded oligonucleotide is RNA or DNA.

50. The polymer strand pair of claim 48, wherein the at least one complementary single-stranded oligonucleotide comprises at least one label monomer.

51. The polymer strand pair of claim 50, wherein the at least one label monomer is selected from the group consisting of a fluorochrome, quantum dot, dye, enzyme, nanoparticle, chemiluminescent marker, biotin, and another monomer that can be detected directly or indirectly.

52. The polymer strand pair of claim 50, wherein an at least one label monomer at a first label attachment position is spectrally or spatially distinguishable from an at least one label monomer at an at least second label attachment position.

53. The polymer strand pair of claim 48, wherein the reporter probe further comprises an affinity moiety.

54. The polymer strand pair of claim 48, wherein the binding portion complementary to the sequence-specific region is about 20 to about 50 nucleotides in length.

55. (canceled)

56. The polymer strand pair of claim 1, wherein the sequence-specific region comprises at least one label monomer selected from the group consisting of a fluorochrome, quantum dot, dye, enzyme, nanoparticle, mass tag, chemiluminescent marker, biotin, and another monomer that can be detected directly or indirectly.

57-58. (canceled)

59. The polymer strand pair of claim 1, wherein the first polymer strand further comprises a cleavable linker between the first complementary region and the sequence-specific region.

60. The polymer strand pair of claim 59, wherein the cleavable linker is photo-cleavable.

61-62. (canceled)

63. The polymer strand pair claim 1, wherein when the first complementary region and the second complementary region are hybridized, the first polymer strand and the second polymer strand form a partially-double stranded nucleic acid probe.

64. The partially double-stranded nucleic acid probe of claim 63.

65. The partially double-stranded nucleic acid probe claim 64, wherein the measured or predicted melting temperature from the first target and the second target is between about 40° C. and about 60° C.

66-67. (canceled)

68. The partially double-stranded nucleic acid probe of claim 64, wherein the measured or predicted melting temperature from the first target is between about 5° C. and about 35° C. and from the second target is between about 5° C. and about 35° C.

69. A composition comprising a plurality of polymer strand pairs of claim 1, wherein a first polymer strand pair is capable of binding to a first nucleic acid molecule and an at least second polymer strand pair is capable of binding to an at least second nucleic acid molecule,
wherein the first nucleic acid molecule differs from the at least second nucleic acid molecule.

70. A polymer strand trio comprising:
(a) a polymer strand pair of claim 1 and
(b) a capture polymer strand at least comprising:
a region comprising at least one affinity moiety or comprising a region capable of binding to a single-stranded nucleic acid comprising at least one affinity moiety and
a third target binding region capable of binding to the nucleic acid molecule,
wherein the targets of the first, second, and third target binding regions are non-overlapping and in the same nucleic acid molecule.

71. A composition comprising a plurality of polymer strand trios of claim 70, wherein a first polymer strand trio is capable of binding to a first nucleic acid molecule and an at least second polymer strand trio is capable of binding to an at least second nucleic acid molecule,
wherein the first nucleic acid molecule differs from the at least second nucleic acid molecule.

72. A composition comprising a plurality of partially double-stranded nucleic acid probes of claim 64.

73. (canceled)

74. A method for detecting a nucleic acid in a sample comprising:
(1) contacting the sample with a polymer strand pair of claim 1, wherein
(a) the sequence-specific region comprises at least two label attachment positions covalently linked in a linear combination, wherein each label attachment position is capable of binding at least one complementary single-stranded oligonucleotide comprising at least one label monomer, wherein a linear combination of labelled monomers identifies the nucleic acid molecule;
(b) the sequence-specific region is covalently attached to a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies the nucleic acid molecule;
(c) the sequence-specific region is bound or capable of being bound to a reporter probe, the reporter probe comprising at least a binding portion complementary to the sequence-specific region and a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies the nucleic acid molecule; or
(d) the sequence-specific region comprises one or more label monomers, wherein the one or more label monomers identifies the nucleic acid molecule;
wherein the at least one label monomer and the one or more label monomers are selected from the group consisting of a fluorochrome, quantum dot, dye, enzyme, nanoparticle, mass tag, chemiluminescent marker, biotin, and another monomer that can be detected directly or indirectly;
(2) detecting the linear combination of labelled monomers or the one or more label monomers, thereby detecting the nucleic acid molecule in the sample.

75. (canceled)

76. A method for detecting a plurality of nucleic acids in a sample comprising:
(1) contacting the sample with a first polymer strand pair and an at least second polymer strand pair of claim 1, wherein
(a) each sequence-specific region comprises at least two label attachment positions covalently linked in a linear combination, wherein each label attachment position is capable of binding at least one complementary single-stranded oligonucleotide comprising at least one label monomer, wherein a linear combination of labelled monomers identifies either a first nucleic acid molecule or an at least second nucleic acid molecule;
(b) each sequence-specific region is covalently attached to a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies either the first nucleic acid molecule or the at least second nucleic acid molecule;
(c) the sequence-specific region is bound or capable of being bound to a reporter probe, the reporter probe comprising at least a binding portion complementary to the sequence-specific region and a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies either the first nucleic acid molecule or the at least second nucleic acid molecule; or
(d) the sequence-specific region comprises one or more label monomers, wherein the one or more label monomers identifies the first nucleic acid molecule or the at least second nucleic acid molecule;
wherein the at least one label monomer and the one or more label monomers are selected from the group consisting of a fluorochrome, quantum dot, dye, enzyme, nanoparticle, mass tag, chemiluminescent marker, biotin, and another monomer that can be detected directly or indirectly;
(2) detecting the linear combination of labelled monomers or the one or more label monomers for the first polymer strand pair and for the at least second polymer strand pair, thereby detecting the first nucleic acid molecule and the at least second nucleic acid molecule in the sample.

77. (canceled)

78. A method for detecting a nucleic acid in a sample comprising:
(1) contacting the sample with the partially double-stranded nucleic acid probe of claim 64, wherein
(a) the sequence-specific region comprises at least two label attachment positions covalently linked in a linear combination, wherein each label attachment position is capable of binding at least one complementary single-stranded oligonucleotide comprising at least one label monomer, wherein a linear combination of labelled monomers identifies the nucleic acid molecule;
(b) the sequence-specific region is covalently attached to a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies the nucleic acid molecule;
(c) the sequence-specific region is bound or capable of being bound to a reporter probe, the reporter probe comprising at least a binding portion complementary to the sequence-specific region and a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies the nucleic acid molecule; or
(d) the sequence-specific region comprises one or more label monomers, wherein the one or more label monomers identifies the nucleic acid molecule;
wherein the at least one label monomer and the one or more label monomers are selected from the group consisting of a fluorochrome, quantum dot, dye, enzyme, nanoparticle, mass tag, chemiluminescent marker, biotin, and another monomer that can be detected directly or indirectly;
(2) detecting the linear combination of labelled monomers or the one or more label monomers, thereby detecting the nucleic acid molecule in the sample.

79. (canceled)

80. A method for detecting a plurality of nucleic acids in a sample comprising:
(1) contacting the sample with a first partially double-stranded nucleic acid probe and an at least second partially double-stranded nucleic acid probe of claim 64, wherein
(a) each sequence-specific region comprises at least two label attachment positions covalently linked in a linear combination, wherein each label attachment position is capable of binding at least one complementary single-stranded oligonucleotide comprising at least one label monomer, wherein a linear combination of labelled monomers identifies either a first nucleic acid molecule or an at least second nucleic acid molecule;
(b) each sequence-specific region is covalently attached to a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies either the first nucleic acid molecule or the at least second nucleic acid molecule;
(c) the sequence-specific region is bound or capable of being bound to a reporter probe, the reporter probe comprising at least a binding portion complementary to the sequence-specific region and a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies either the first nucleic acid molecule or the at least second nucleic acid molecule; or
(d) the sequence-specific region comprises one or more label monomers, wherein the one or more label monomers identifies the first nucleic acid molecule or the at least second nucleic acid molecule;
wherein the at least one label monomer and the one or more label monomers are selected from the group consisting of a fluorochrome, quantum dot, dye, enzyme, nanoparticle, mass tag, chemiluminescent marker, biotin, and another monomer that can be detected directly or indirectly;
(2) detecting the linear combination of labelled monomers or the one or more label monomers for the first polymer strand pair and for the at least second polymer strand pair, thereby detecting the first nucleic acid molecule and the at least second nucleic acid molecule in the sample.

81. (canceled)

82. A method for detecting a nucleic acid in a sample comprising:
(1) contacting the sample with a polymer strand trio of claim 70, wherein
(a) the sequence-specific region comprises at least two label attachment positions covalently linked in a linear combination, wherein each label attachment position is capable of binding at least one complementary single-stranded oligonucleotide comprising at least one label monomer, wherein a linear combination of labelled monomers identifies the nucleic acid molecule;
(b) the sequence-specific region is covalently attached to a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies the nucleic acid molecule;
(c) the sequence-specific region is bound or capable of being bound to a reporter probe, the reporter probe comprising at least a binding portion complementary to the sequence-specific region and a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies the nucleic acid molecule; or
(d) the sequence-specific region comprises one or more label monomers, wherein the one or more label monomers identifies the nucleic acid molecule;
wherein the at least one label monomer and the one or more label monomers are selected from the group consisting of a fluorochrome, quantum dot, dye, enzyme, nanoparticle, mass tag, chemiluminescent marker, biotin, and another monomer that can be detected directly or indirectly;
(2) detecting the linear combination of labelled monomers or the one or more label monomers, thereby detecting the nucleic acid molecule in the sample.

83. A method for detecting a plurality of nucleic acids in a sample comprising:
(1) contacting the sample with a first polymer strand trio and an at least second polymer strand trio of claim 70, wherein
(a) each sequence-specific region comprises at least two label attachment positions covalently linked in a linear combination, wherein each label attachment position is capable of binding at least one complementary single-stranded oligonucleotide comprising at least one label monomer, wherein a linear combination of labelled monomers identifies either a first nucleic acid molecule or an at least second nucleic acid molecule;
(b) each sequence-specific region is covalently attached to a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies either the first nucleic acid molecule or the at least second nucleic acid molecule;
(c) the sequence-specific region is bound or capable of being bound to a reporter probe, the reporter probe comprising at least a binding portion complementary to the sequence-specific region and a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies either the first nucleic acid molecule or the at least second nucleic acid molecule; or
(d) the sequence-specific region comprises one or more label monomers, wherein the one or more label monomers identifies the first nucleic acid molecule or the at least second nucleic acid molecule;
wherein the at least one label monomer and the one or more label monomers are selected from the group consisting of a fluorochrome, quantum dot, dye, enzyme, nanoparticle, mass tag, chemiluminescent marker, biotin, and another monomer that can be detected directly or indirectly;
(2) detecting the linear combination of labelled monomers or the one or more label monomers for the first polymer strand trio and for the at least second polymer strand trio, thereby detecting the first nucleic acid molecule and the at least second nucleic acid molecule in the sample.

84. A multivalent polymer strand comprising at least:
a first target binding region,
a second target binding region,
a spacer between the first target binding region and the second target binding region; and
a sequence-specific region;
wherein the target of the first target binding region and the target of the second target binding region are in the same nucleic acid molecule and the target of the first target binding region is non-overlapping with the target of the second target binding region.

85-140. (canceled)

141. A composition comprising a plurality of multivalent polymer strands of claim 84, wherein a first multivalent polymer strand is capable of binding to a first nucleic acid molecule and an at least second multivalent polymer strand is capable of binding to an at least second nucleic acid molecule,
wherein the first nucleic acid molecule differs from the at least second nucleic acid molecule.

142-143. (canceled)

144. A method for detecting a nucleic acid in a sample comprising:
(1) contacting the sample with a multivalent polymer strand of claim 84, wherein
(a) the sequence-specific region comprises at least two label attachment positions covalently linked in a linear combination, wherein each label attachment position is capable of binding at least one complementary single-stranded oligonucleotide comprising at least one label monomer, wherein a linear combination of labelled monomers identifies the nucleic acid molecule;
(b) the sequence-specific region is covalently attached to a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies the nucleic acid molecule;
(c) the sequence-specific region is bound or capable of being bound to a reporter probe, the reporter probe comprising at least a binding portion complementary to the sequence-specific region and a single-stranded nucleic acid backbone, the backbone comprising at least two label attachment positions covalently linked in a linear combination, wherein each label attachment position is capable of binding at least one complementary single-stranded oligonucleotide comprising at least one label monomer, wherein a linear combination of labelled monomers identifies the nucleic acid molecule; or
(d) the sequence-specific region comprises one or more label monomers, wherein the one or more label monomers identifies the nucleic acid molecule;
wherein the at least one label monomer and the one or more label monomers are selected from the group consisting of a fluorochrome, quantum dot, dye, enzyme, nanoparticle, mass tag, chemiluminescent marker, biotin, and another monomer that can be detected directly or indirectly;
(2) detecting the linear combination of labelled monomers or the one or more label monomers, thereby detecting the nucleic acid molecule in the sample.

145-149. (canceled)

150. A kit comprising a composition of any of claim 69 and instructions for use.

151. The kit of claim 150 further comprising at least one probe capable of detecting a protein target.

152. The composition of any claim 69 further comprising at least one probe capable of detecting a protein target.

153. The method of any claim 74 further comprising contacting the sample with at least one probe capable of detecting a protein target.

154-155. (canceled)
